SEQUENCE LISTING (20 sequences)

SEQ ID NO: 1 - DNA, 1719 bp, synthetic construct
  source 1..1719: mol_type = other DNA
atggccatctccggcgtgcccgtgctgggcttcttcatcatcgccgtgctgatgtccgcccaggagtcctgggccgaccagatctgcatcggctaccactccaacaactccacccagaccgtgaacaccctgctggagtccaacgtgcccgtgacctcctcccactccatcctggagaaggagcacaacggcctgctgtgcaagctgaagggcaaggcccccctggacctgatcgactgctccctgcccgcctggctgatgggcaaccccaagtgcgacgagctgctgaccgcctccgagtgggcctacatcaaggaggaccccgagcccgagaacggcatctgcttccccggcgacttcgactccctggaggacctgatcctgctggtgtccaacaccgaccacttccgcaaggagaagatcatcgacatgacccgcttctccgacgtgaccaccaacaacgtggactccgcctgcccctacgacaccaacggcgcctccttctaccgcaacctgaactgggtgcagcagaacaagggcaagcagctgatcttccactaccagaactccgagaacaaccccctgctgatcatctggggcgtgcaccagacctccaacgccgccgagcagaacacctactacggctcccagaccggctccaccaccatcaccatcggcgaggagaccaacacctaccccctggtgatctccgagtcctccatcctgaacggccactccgaccgcatcaactacttctggggcgtggtgaaccccaaccagaacttctccatcgtgtccaccggcaacttcatctggcccgagtacggctacttcttccagaagaccaccaacatctccggcatcatcaagtcctccgagaagatctccgactgcgacaccatctgccagaccaagatcggcgccatcaactccaccctgcccttccagaacatccaccagaacgccatcggcgactgccccaagtacgtgaaggcccaggagctggtgctggccaccggcctgcgcaacaaccccatcaaggagacccgcggcctgttcggcgccatcgccggcttcatcgagggcggctggcagggcctgatcgacggctggtacggctaccaccaccagaactccgagggctccggctacgccgccgacaaggaggccacccagaaggccgtggacgccatcaccaccaaggtgaacaacatcatcgacaagatgaacacccagttcgagtccaccgccaaggagttcaacaagatcgagatgcgcatcaagcacctgtccgaccgcgtggacgacggcttcctggacgtgtggtcctacaacgccgagctgctggtgctgctggagaacgagcgcaccctggacttccacgacgccaacgtgaacaacctgtaccagaaggtgaaggtgcagctgaaggacaacgccatcgacatgggcaacggctgcttcaagatcctgcacaagtgcaacaacacctgcatggacgacatcaagaacggcacctacaactactacgagtaccgcaaggagtcccacctggagaagcagaagatcgacggcgtgaagctgtccgagaactcctcctacaagatcatgatcatctactccaccgtggcctcctccgtggtgctgggcctgatcatcctggccgccatcgagtggggctgcttcaagggcaacctgcagtgccgcatctgcatctaa

SEQ ID NO: 2 - DNA, 1719 bp, synthetic construct
  source 1..1719: mol_type = other DNA
atggccatctccggcgtgcccgtgctgggcttcttcatcatcgccgtgctgatgtccgcccaggagtcctgggccgacaagatctgcatcggctacctgtccaacaacgccaccgacaccgtggacaccctgaccgagaacggcgtgcccgtgacctcctccgtggacctggtggagaccaaccacaccggcacctactgctccctgaacggcatctcccccatccacctgggcgactgctccttcgagggctggatcgtgggcaacccctcctgcgccaccaacatcaacatccgcgagtggtcctacctgatcgaggaccccaacgcccccaacaagctgtgcttccccggcgagctggacaacaacggcgagctgcgccacctgttctccggcgtgaactccttctcccgcaccgagctgatctcccccaacaagtggggcgacatcctggacggcgtgaccgcctcctgccgcgacaacggcgcctcctccttctaccgcaacctggtgtggatcgtgaagaacaagaacggcaagtaccccgtgatcaagggcgactacaacaacaccaccggccgcgacgtgctggtgctgtggggcatccaccaccccgacaccgagaccaccgccatcaacctgtacgcctccaagaacccctacaccctggtgtccaccaaggagtggtccaagcggtacgagctggagatcggcacccgcatcggcgacggccagcggtcctggatgaagctgtactggcacctgatgcgccccggcgagcgcatcatgttcgagtccaacggcggcctgatcgccccccgctacggctacatcatcgagaagtacggcaccggccgcatcttccagtccggcgtgcgcatggccaagtgcaacaccaagtgccagacctccctgggcggcatcaacaccaacaagaccttccagaacatcgagcgcaacgccctgggcgactgccccaagtacatcaagtccggccagctgaagctggccaccggcctgcgcaacgtgccctccgtgggcgagcgcggcctgttcggcgccatcgccggcttcatcgagggcggctggcccggcctgatcaacggctggtacggcttccagcaccagaacgagcagggcaccggcatcgccgccgacaaggcctccacccagaaggccatcgacgagatcaccaccaagatcaacaacatcatcgagaagatgaacggcaactacgactccatccgcggcgagttcaaccaggtggagaagcgcatcaacatgctggccgaccgcgtggacgacgccgtgaccgacatctggtcctacaacgccaagctgctggtgctgctggagaacggccgcaccctggacctgcacgacgccaacgtgcgcaacctgcacgaccaggtgaagcgcatcctgaagtccaacgccatcgacgagggcgacggctgcttcaacctgctgcacaagtgcaacgactcctgcatggagaccatccgcaacggcacctacaaccacgaggactaccgcgaggagtcccagctgaagcgccaggagatcgagggcatcaagctgaagtccgaggacaacgtgtacaaggtgctgtccatctactcctgcatcgcctcctccatcgtgctggtgggcctgatcctggccttcatcatgtgggcctgctccaacggcaactgccgcttcaacgtgtgcatctaa

SEQ ID NO: 3 - DNA, 1722 bp, synthetic construct
  source 1..1722: mol_type = other DNA
atggccatctccggcgtgcccgtgctgggcttcttcatcatcgccgtgctgatgtccgcccaggagtcctgggcccagaactacaccggcaaccccgtgatctgcatgggccaccacgccgtggccaacggcaccatggtgaagaccctggccgacgaccaggtggaggtggtgaccgcccaggagctggtggagtcccagaacctgcccgagctgtgcccctcccccctgcgcctggtggacggccagacctgcgacatcatcaacggcgccctgggctcccccggctgcgaccacctgaacggcgccgagtgggacgtgttcatcgagcgccccaacgccgtggacacctgctaccccttcgacgtgcccgagtaccagtccctgcgctccatcctggccaacaacggcaagttcgagttcatcgccgaggagttccagtggaacaccgtgaagcagaacggcaagtccggcgcctgcaagcgcgccaacgtgaacgacttcttcaaccgcctgaactggctggtgaagtccgacggcaacgcctaccccctgcagaacctgaccaagatcaacaacggcgactacgcccgcctgtacatctggggcgtgcaccacccctccaccgacaccgagcagaccaacctgtacaagaacaaccccggcggcgtgaccgtgtccaccaagacctcccagacctccgtggtgcccaacatcggctcccgccccctggtgcgcggccagtccggccgcgtgtccttctactggaccatcgtggagcccggcgacctgatcgtgttcaacaccatcggcaacctgatcgccccccgcggccactacaagctgaacaaccagaagaagtccaccatcctgaacaccgccatccccatcggctcctgcgtgtccaagtgccacaccgacaagggctccctgtccaccaccaagcccttccagaacatctcccgcatcgccgtgggcgactgcccccgctacgtgaagcagggctccctgaagctggccaccggcatgcgcaacatccccgagaaggcctcccgcggcctgttcggcgccatcgccggcttcatcgagaacggctggcagggcctgatcgacggctggtacggcttccgccaccagaacgccgagggcaccggcaccgccgccgacctgaagtccacccaggccgccatcgaccagatcaacggcaagctgaaccgcctgatcgagaagaccaacgacaagtaccaccagatcgagaaggagttcgagcaggtggagggccgcatccaggacctggagaagtacgtggaggacaccaagatcgacctgtggtcctacaacgccgagctgctggtggccctggagaaccagcacaccatcgacgtgaccgactccgagatgaacaagctgttcgagcgcgtgcgccgccagctgcgcgagaacgccgaggacaagggcaacggctgcttcgagatcttccacaagtgcgacaacaactgcatcgagtccatccgcaacggcacctacgaccacgacatctaccgcgacgaggccatcaacaaccgcttccagatccagggcgtgaagctgacccagggctacatggacatcatcctgtggatctccttctccatctcctgcttcctgctggtggccctgctgctggccttcatcctgtgggcctgccagaacggcaacatccgctgccagatctgcatctaa

SEQ ID NO: 4 - DNA, 1704 bp, synthetic construct
  source 1..1704: mol_type = other DNA
atggccatctccggcgtgcccgtgctgggcttcttcatcatcgccgtgctgatgtccgcccaggagtcctgggccgacaagatctgcatcggccaccagtccaccaactccaccgagaccgtggacaccctgaccgagaccaacgtgcccgtgacccacgccaaggagctgctgcagaccgagcacaacggcatgctgtgcgccaccaacctgggccaccccctgatcctggacacctgcaccatcgagggcctgatctacggcaacccctcctgcgacctgctgctggagggccgcgagtggtcctacatcgtggagcgcccctccgccgtgaacggcacctgctaccccggcaacgtggagaacctggaggagctgcgcaccctgttctcctccgcctcctcctaccagcgcatccagatcttccccgacaccacctggaacgtgacctacaccggcacctcccgctcctgctccggctccttctaccgctccatgcgctggctgacccagaagtccggcttctaccccgtgcaggacgccaagtacaccaacaaccgcggcaagaacatcctgttcgtgtggggcatccaccacccccacacctacaccgaccagaccaccctgtacatccgcaacgacaccaccacctccgtgaccaccgaggagctgaaccgcatcttcaagcccgtgatcgtgccccgcctgctggtgaacggccagcagggccgcatcgactactactggtccgtgctgaagcccggccagaccctgcgcgtgcgctccaacggcaacctgatcgccccctggtacggccacgtgctgtccggcggctcccacggccgcatcctgaagaccgacctgaagtccggcaactgcgtggtgcagtgccagaccgagaagggcggcctgaactccaccctgcccttccacaacatctccaagtacgccttcggcacctgccccaagtacgtgaaggtgaactccctgaagctggccatcggcctgcgcaacgtgcccgcccgctccaaccgcggcctgttcggcgccatcgccggcttcatcgagggcggctggcccggcctggtggccggctggtacggcttccagcactccaacgaccagggcgtgggcatggccgccgaccgcgactccacccagaaggccgtggacaagatcacctccaaggtgaacaacatcgtggacaagatgaacaagcagtacgagatcatcgaccacgagttctccgaggtggagacccgcctgaacatgatcaacaacaagatcgacgaccagatccaggacgtgtgggcctacaacgccgagctgctggtgctgctggagaaccagaagaccctggacgagcacgacgccaacgtgaacaacctgtacaacaaggtgaagcgcgccctgggctccaacgccatggaggacggcaagggctgcttcgagctgtaccacaagtgcgacgaccagtgcatggagaccatccgcaacggcacctacgaccgccgcaagtaccgcgaggagtcccgcctggagcgccagcgcatcgagggcgtgaagctggagtccgagggcacctacaagatcctgaccatctactccaccgtggcctcctccctggtgatcgccatgggcttcgccgccttcctgttctgggccatgtccaacggctcctgccgctgcaacatctgcatctaa

SEQ ID NO: 5 - DNA, 1719 bp, synthetic construct
  source 1..1719: mol_type = other DNA
atggccatctccggcgtgcccgtgctgggcttcttcatcatcgccgtgctgatgtccgcccaggagtcctgggccgaccagatctgcatcggctaccacgccaacaactccaccgagaaggtggacaccatcctggagcgcaacgtgaccgtgacccacgccaaggacatcctggagaagacccacaacggcaagctgtgcaagctgaacggcatcccccccctggagctgggcgactgctccatcgccggctggctgctgggcaaccccgagtgcgaccgcctgctgtccgtgcccgagtggtcctacatcatggagaaggagaacccccgcgacggcctgtgctaccccggctccttcaacgactacgaggagctgaagcacctgctgtcctccgtgaagcacttcgagaaggtgaagatcctgcccaaggaccgctggacccagcacaccaccaccggcggctcccgcgcctgcgccgtgtccggcaacccctccttcttccgcaacatggtgtggctgaccaagaagggctccaactaccccgtggccaagggctcctacaacaacacctccggcgagcagatgctgatcatctggggcgtgcaccaccccaacgacgagaccgagcagcgcaccctgtaccagaacgtgggcacctacgtgtccgtgggcacctccaccctgaacaagcggtccacccccgagatcgccacccgccccaaggtgaacggcctgggcggccgcatggagttctcctggaccctgctggacatgtgggacaccatcaacttcgagtccaccggcaacctgatcgcccccgagtacggcttcaagatctccaagcgcggctcctccggcatcatgaagaccgagggcaccctggagaactgcgagaccaagtgccagacccccctgggcgccatcaacaccaccctgcccttccacaacgtgcaccccctgaccatcggcgagtgccccaagtacgtgaagtccgagaagctggtgctggccaccggcctgcgcaacgtgccccagatcgagtcccgcggcctgttcggcgccatcgccggcttcatcgagggcggctggcagggcatggtggacggctggtacggctaccaccactccaacgaccagggctccggctacgccgccgacaaggagtccacccagaaggccttcgacggcatcaccaacaaggtgaactccgtgatcgagaagatgaacacccagttcgaggccgtgggcaaggagttctccaacctggagcgccgcctggagaacctgaacaagaagatggaggacggcttcctggacgtgtggacctacaacgccgagctgctggtgctgatggagaacgagcgcaccctggacttccacgactccaacgtgaagaacctgtacgacaaggtgcgcatgcagctgcgcgacaacgtgaaggagctgggcaacggctgcttcgagttctaccacaagtgcgacgacgagtgcatgaactccgtgaagaacggcacctacgactaccccaagtacgaggaggagtccaagctgaaccgcaacgagatcaagggcgtgaagctgtcctccatgggcgtgtaccagatcctggccatctacgccaccgtggccggctccctgtccctggccatcatgatggccggcatctccttctggatgtgctccaacggctccctgcagtgccgcatctgcatctaa

SEQ ID NO: 6 - DNA, 1713 bp, synthetic construct
  source 1..1713: mol_type = other DNA
atggccatctccggcgtgcccgtgctgggcttcttcatcatcgccgtgctgatgtccgcccaggagtcctgggccctggacaagatctgcctgggccaccacgccgtggccaacggcaccatcgtgaagaccctgaccaacgagcaggaggaggtgaccaacgccaccgagaccgtggagtccaccggcatcaaccgcctgtgcatgaagggccgcaagcacaaggacctgggcaactgccaccccatcggcatgctgatcggcacccccgcctgcgacctgcacctgaccggcacctgggacaccctgatcgagcgcgagaacgccatcgcctactgctaccccggcgccaccgtgaacgtggaggccctgcgccagaagatcatggagtccggcggcatcgacaagatctccaccggcttcacctacggctcctccatcaactccgccggcaccacccgcgcctgcatgcgcaacggcggcaactccttctacgccgagctgaagtggctggtgtccaagtccaagggccaggacttcccccagaccaccaacacctaccgcaacaccgacaccgccgagcacctgatcatgtggggcatccaccacccctcctccatccaggagaagaacgacctgtacggcacccagtccctgtccatctccgtgggctcctccacctaccgcaacaacttcgtgcccgtggtgggcgcccgcccccaggtgaacggccagtccggccgcatcgacttccactggaccctggtgcagcccggcgacaacatcaccttctcccacaacggcggcctgatcgccccctcccgcgtgtccaagctgatcggccgcggcctgggcatccagtccgacgcccccatcgacaacaactgcgagtccaagtgcttctggcgcggcggctccatcaacacccgcctgcccttccagaacctgtccccccgcaccgtgggccagtgccccaagtacgtgaaccgccgctccctgatgctggccaccggcatgcgcaacgtgcccgagctgatccagggccgcggcctgttcggcgccatcgccggcttcctggagaacggctgggagggcatggtggacggctggtacggcttccgccaccagaacgcccagggcaccggccaggccgccgactacaagtccacccaggccgccatcgaccagatcaccggcaagctgaaccgcctggtggagaagaccaacaccgagttcgagtccatcgagtccgagttctccgagatcgagcaccagatcggcaacgtgatcaactggaccatggactccatcaccgacatctggacctaccaggccgagctgctggtggccatggagaaccagcacaccatcgacatggccgactccgagatgctgaacctgtacgagcgcgtgcgcaagcagctgcgccagaacgccgaggaggacggcaagggctgcttcgagatctaccacgcctgcgacgactcctgcatggagtccatccgcaacaacacctacgaccactcccagtaccgcgaggaggccctgctgaaccgcctgaacatcaaccccgtgaccctgtcctccggctacaaggacatcatcctgtggttctccttcggcgcctcctgcttcgtgctgctggccgtggtgatgggcctggtgttcttctgcctgaagaacggcaacatgcgctgcaccatctgcatctaa

SEQ ID NO: 7 - DNA, 1731 bp, synthetic construct
  source 1..1731: mol_type = other DNA
atggccatctccggcgtgcccgtgctgggcttcttcatcatcgccgtgctgatgtccgcccaggagtcctgggcccagatcaccaacggcaccaccggcaaccccatcatctgcctgggccaccacgccgtggagaacggcacctccgtgaagaccctgaccgacaaccacgtggaggtggtgtccgccaaggagctggtggagaccaaccacaccgacgagctgtgcccctcccccctgaagctggtggacggccaggactgccacctgatcaacggcgccctgggctcccccggctgcgaccgcctgcaggacaccacctgggacgtgttcatcgagcgccccaccgccgtggacacctgctaccccttcgacgtgcccgactaccagtccctgcgctccatcctggcctcctccggctccctggagttcatcgccgagcagttcacctggaacggcgtgaaggtggacggctcctcctccgcctgcctgcgcggcggccgcaactccttcttctcccgcctgaactggctgaccaaggccaccaacggcaactacggccccatcaacgtgaccaaggagaacaccggctcctacgtgcgcctgtacctgtggggcgtgcaccacccctcctccgacaacgagcagaccgacctgtacaaggtggccaccggccgcgtgaccgtgtccacccgctccgaccagatctccatcgtgcccaacatcggctcccgcccccgcgtgcgcaaccagtccggccgcatctccatctactggaccctggtgaaccccggcgactccatcatcttcaactccatcggcaacctgatcgccccccgcggccactacaagatctccaagtccaccaagtccaccgtgctgaagtccgacaagcgcatcggctcctgcacctccccctgcctgaccgacaagggctccatccagtccgacaagcccttccagaacgtgtcccgcatcgccatcggcaactgccccaagtacgtgaagcagggctccctgatgctggccaccggcatgcgcaacatccccggcaagcaggccaagggcctgttcggcgccatcgccggcttcatcgagaacggctggcagggcctgatcgacggctggtacggcttccgccaccagaacgccgagggcaccggcaccgccgccgacctgaagtccacccaggccgccatcgaccagatcaacggcaagctgaaccgcctgatcgagaagaccaacgagaagtaccaccagatcgagaaggagttcgagcaggtggagggccgcatccaggacctggagaagtacgtggaggacaccaagatcgacctgtggtcctacaacgccgagctgctggtggccctggagaaccagcacaccatcgacgtgaccgactccgagatgaacaagctgttcgagcgcgtgcgccgccagctgcgcgagaacgccgaggaccagggcaacggctgcttcgagatcttccaccagtgcgacaacaactgcatcgagtccatccgcaacggcacctacgaccacaacatctaccgcgacgaggccatcaacaaccgcatcaagatcaaccccgtgaccctgaccatgggctacaaggacatcatcctgtggatctccttctccatgtcctgcttcgtgttcgtggccctgatcctgggcttcgtgctgtgggcctgccagaacggcaacatccgctgccagatctgcatctaa

SEQ ID NO: 8 - DNA, 1722 bp, synthetic construct
  source 1..1722: mol_type = other DNA
atggccatctccggcgtgcccgtgctgggcttcttcatcatcgccgtgctgatgtccgcccaggagtcctgggcctacgacaagatctgcatcggctaccagaccaacaactccaccgagaccgtgaacaccctgatcgagcagaacgtgcccgtgacccaggtggaggagctggtgcacggcggcatcgaccccgtgctgtgcggcaccgagctgggctcccccctggtgctggacgactgctccctggagggcctgatcctgggcaaccccaagtgcgacctgtacctgaacggccgcgagtggtcctacatcgtggagcgccccaaggagatggagggcgtgtgctaccccggctccatcgagaaccaggaggagctgcgctccctgttctcctccatcaagaagtacgagcgcgtgaagatgttcgacttcaccaagtggaacgtgacctacaccggcacctccaaggcctgcaacaacacctccaaccagggctccttctaccgctccatgcgctggctgaccctgaagtccggccagttccccgtgcagaccgacgagtacaagaacacccgcgactccgacatcgtgttcacctgggccatccaccacccccccacctccgacgagcagatcaagctgtacaagaaccccgacaccctgtcctccgtgaccaccgacgagatcaaccgctccttcaagcccaacatcggcccccgccccctggtgcgcggccagcagggccgcatggactactactgggccgtgctgaagcccggccagaccgtgaagatccagaccaacggcaacctgatcgcccccgagtacggccacctgatcaccggcaagtcccacggccgcatcctgaagaacaacctgcccatcggccagtgcgtgaccgagtgccagctgaacgagggcgtgatgaacacctccaagcccttccagaacacctccaagcactacatcggcaagtgccccaagtacatcccctccggctccctgaagctggccatcggcctgcgcaacgtgccccaggtgcagaaccgcggcctgttcggcgccaaggccggcttcatcgagggcggctggcccggcctggtggccggctggtacggcttccagcaccagaacgccgagggcaccggcatcgccgccgaccgcgactccacccagaaggccatcgacaacatgcagaacaagctgaacaacgtgatcgacaagatgaacaagcagttcgaggtggtgaaccacgagttctccgaggtggagtcccgcatcaacatgatcaactcccagatcgacgaccagatcaccgacatctgggcctacaacgccgagctgctggtgctgctggagaaccagaagaccctggacgagcacgacgccaacgtgcgcaacctgcacgaccgcgtgcgccgcgtgctgcgcgagaacgccatcgacaccggcgacggctgcttcgagatcctgcacaagtgcgacaacaactgcatggacaccatccgcaacggcacctacaaccacaaggagtacgaggaggagtccaagatcgagcgccagaagatcaacggcgtgaagctggaggagaactccacctacaagatcctgtccatctactcctccgtggcctcctccctggtgctgctgctgatgatcatcggcggcttcatcttcggctgccagaacggcaacgtgcgctgcaccttctgcatctaa

SEQ ID NO: 9 - DNA, 1728 bp, synthetic construct
  source 1..1728: mol_type = other DNA
atggccatctccggcgtgcccgtgctgggcttcttcatcatcgccgtgctgatgtccgcccaggagtcctgggcctacgaccgcatctgcatcggctaccagtccaacaactccaccgacaccgtgaacaccctgatcgagcagaacgtgcccgtgacccagaccatggagctggtggagaccgagaagcaccccgcctactgcaacaccgacctgggcgcccccctggagctgcgcgactgcaagatcgaggccgtgatctacggcaaccccaagtgcgacatccacctgaaggaccagggctggtcctacatcgtggagcgcccctccgcccccgagggcatgtgctaccccggctccgtggagaacctggaggagctgcgcttcgtgttctcctccgccgcctcctacaagcgcatccgcctgttcgactactcccgctggaacgtgacccgctccggcacctccaaggcctgcaacgcctccaccggcggccagtccttctaccgctccatcaactggctgaccaagaagaagcccgacacctacgacttcaacgagggcacctacatcaacaacgaggacggcgacatcatcttcctgtggggcatccaccacccccccgacgccaaggagcagaccaccctgtacaagaacgccaacaccctgtcctccgtgaccaccaacaccatcaaccgctccttccagcccaacatcggcccccgccccctggtgcgcggccagcagggccgcatggactactactggggcatcctgaagcgcggcgagaccctgaagatccgcaccaacggcaacctgatcgcccccgagttcggctacctgctgaagggcgagtcccacggccgcatcatccagaacgaggacatccccatcggctcctgccacaccaagtgccagacctacgccggcgccatcaactcctccaagcccttccagaacgcctcccgccactacatgggcgagtgccccaagtacgtgaagaaggagtccctgcgcctggccgtgggcctgcgcaacaccccctccatcgagccccgcggcctgttcggcgccatcgccggcttcatcgagggcggctggtccggcatgatcgacggctggtacggcttccaccactccaactccgagggcaccggcatggccgccgaccagaagtccacccaggaggccatcgacaagatcaccaacaaggtgaacaacatcgtggacaagatgaaccgcgagttcgaggtggtgaaccacgagttctccgaggtggagaagcgcatcaacatgatcaacgacaagatcgacgaccagatcgaggacctgtgggcctacaacgccgagctgctggtgctgctggagaaccagaagaccctggacgagcacgactccaacgtgaagaacctgttcgacgaggtgcgccgccgcctgtccgccaacgccatcgacaccggcaacggctgcttcgacatcctgcacaagtgcgacaacgagtgcatggagaccatcaagaacggcacctacaaccacaaggagtacgaggaggaggccaagctggagcggtccaagatcaacggcgtgaagctggaggagaacaccacctacaagatcctgtccatctactccaccgtggccgcctccctgtgcctggccatcctgatcgccggcggcctgatcctgggcatgcagaacggctcctgccgctgcatgttctgcatctaa

SEQ ID NO: 10 - DNA, 1725 bp, synthetic construct
  source 1..1725: mol_type = other DNA
atggccatctccggcgtgcccgtgctgggcttcttcatcatcgccgtgctgatgtccgcccaggagtcctgggccgacaccctgtgcatcggctaccacgccaacaactccaccgacaccgtggacaccgtgctggagaagaacgtgaccgtgacccactccgtgaacctgctggaggacaagcacaacggcaagctgtgcaagctgcgcggcgtggcccccctgcacctgggcaagtgcaacatcgccggctggatcctgggcaaccccgagtgcgagtccctgtccaccgcctcctcctggtcctacatcgtggagacctccaactccgacaacggcacctgctaccccggcgacttcatcaactacgaggagctgcgcgagcagctgtcctccgtgtcctccttcgagcggttcgagatcttccccaagacctcctcctggcccaaccacgactccaacaagggcgtgaccgccgcctgcccccacgccggcgccaagtccttctacaagaacctgatctggctggtgaagaagggcaactcctaccccaagctgaaccagtcctacatcaacgacaagggcaaggaggtgctggtgctgtggggcatccaccacccctccaccaccgccgaccagcagtccctgtaccagaacgccgacgcctacgtgttcgtgggcacctcccgctactccaagaagttcaagcccgagatcgccacccgccccaaggtgcgcgaccaggagggccgcatgaactactactggaccctggtggagcccggcgacaagatcaccttcgaggccaccggcaacctggtggtgccccgctacgccttcaccatggagcgcaacgccggctccggcatcatcatctccgacacccccgtgcacgactgcaacaccacctgccagacccccgagggcgccatcaacacctccctgcccttccagaacatccaccccatcaccatcggcaagtgccccaagtacgtgaagtccaccaagctgcgcctggccaccggcctgcgcaacgtgccctccatccagtcccgcggcctgttcggcgccatcgccggcttcatcgagggcggctggaccggcatggtggacggctggtacggctaccaccaccagaacgagcagggctccggctacgccgccgacctgaagtccacccagaacgccatcgacaagatcaccaacaaggtgaactccgtgatcgagaagatgaacacccagttcaccgccgtgggcaaggagttcaaccacctggagaagcgcatcgagaacctgaacaagaaggtggacgacggcttcctggacatctggacctacaacgccgagctgctggtgctgctggagaacgagcgcaccctggactaccacgactccaacgtgaagaacctgtacgagaaggtgcgcaaccagctgaagaacaacgccaaggagatcggcaacggctgcttcgagttctaccacaagtgcgacaacacctgcatggagtccgtgaagaacggcacctacgactaccccaagtactccgaggaggccaagctgaaccgcgagaagatcgacggcgtgaagctggagtccacccgcatctaccagatcctggccatctactccaccgtggcctcctccctggtgctggtggtgtccctgggcgccatctccttctggatgtgctccaacggctccctgcagtgccgcatctgcatctaa

SEQ ID NO: 11 - DNA, 1734 bp, synthetic construct
  source 1..1734: mol_type = other DNA
atggccatctccggcgtgcccgtgctgggcttcttcatcatcgccgtgctgatgtccgcccaggagtcctgggccgacaagatctgcctgggccaccacgccgtggccaacggcaccaaggtgaacaccctgaccgagcgcggcgtggaggtggtgaacgccaccgagaccgtggagatcaccggcatcgacaaggtgtgcaccaagggcaagaaggccgtggacctgggctcctgcggcatcctgggcaccatcatcggccccccccagtgcgacctgcacctggagttcaaggccgacctgatcatcgagcgccgcaactcctccgacatctgctaccccggccgcttcaccaacgaggaggccctgcgccagatcatccgcgagtccggcggcatcgacaaggagtccatgggcttccgctactccggcatccgcaccgacggcgccacctccgcctgcaagcgcaccgtgtcctccttctactccgagatgaagtggctgtcctcctccatgaacaaccaggtgttcccccagctgaaccagacctaccgcaacacccgcaaggagcccgccctgatcgtgtggggcgtgcaccactcctcctccctggacgagcagaacaagctgtacggcaccggcaacaagctgatcaccgtgggctcctccaagtaccagcagtccttctccccctcccccggcgcccgccccaaggtgaacggccaggccggccgcatcgacttccactggatgctgctggaccccggcgacaccgtgaccttcaccttcaacggcgccttcatcgcccccgaccgcgccaccttcctgcgctccaacgccccctccggcatcgagtacaacggcaagtccctgggcatccagtccgacgcccagatcgacgagtcctgcgagggcgagtgcttctactccggcggcaccatcaactcccccctgcccttccagaacatcgactcccgcgccgtgggcaagtgcccccgctacgtgaagcagtcctccctgcccctggccctgggcatgaagaacgtgcccgagaagatccgcacccgcggcctgttcggcgccatcgccggcttcatcgagaacggctgggagggcctgatcgacggctggtacggcttccgccaccagaacgcccagggccagggcaccgccgccgactacaagtccacccaggccgccatcgaccagatcaccggcaagctgaaccgcctgatcgagaagaccaacaagcagttcgagctgatcgacaacgagttcaccgaggtggagcagcagatcggcaacgtgatcaactggacccgcgactccctgaccgagatctggtcctacaacgccgagctgctggtggccatggagaaccagcacaccatcgacctggccgactccgagatgaacaagctgtacgagcgcgtgcgccgccagctgcgcgagaacgccgaggaggacggcaccggctgcttcgagatcttccaccgctgcgacgaccagtgcatggagtccatccgcaacaacacctacaaccacaccgagtaccgccaggaggccctgcagaaccgcatcatgatcaaccccgtgaagctgtcctccggctacaaggacgtgatcctgtggttctccttcggcgcctcctgcgtgatgctgctggccatcgccatgggcctgatcttcatgtgcgtgaagaacggcaacctgcgctgcaccatctgcatctaa

SEQ ID NO: 12 - DNA, 1719 bp, synthetic construct
  source 1..1719: mol_type = other DNA
atggccatctccggcgtgcccgtgctgggcttcttcatcatcgccgtgctgatgtccgcccaggagtcctgggccgaccgcatctgcgtgggctacctgtccaccaactcctccgagaaggtggacaccctgctggagaacggcgtgcccgtgacctcctccgtggacctggtggagaccaaccacaccggcacctactgctccctgaacggcatctcccccgtgcacctgggcgactgctccttcgagggctggatcgtgggcaacccctcctgcgcctccaacctgggcatccgcgagtggtcctacctgatcgaggacccctccgccccccacggcctgtgctaccccggcgagctggacaacaacggcgagctgcgccacctgttctccggcatcaagtccttctcccgcaccgagctgatcgcccccacctcctggggcgccgtgaacgacggcgtgtcctccgcctgccaggacaagggcgcctcctccttctaccgcaacctggtgtggttcgtggagcgcggcaagaagtaccccgtgatccgcggcacctacaacaacaccaccggccgcgacgtgctggtgatgtggggcatccaccaccccgtgtccgaggacgaggcccgcaagctgtacatcaactccaacccctacaccctggtgtccaccggctcctggtccaagaagtacaacctggagatcggcatccgccccggctacaacggccagaagtcctggatgaagatctactggtccctgatgcaccccggcgagtccatctccttcgagtccaacggcggcctgctggccccccgctacggctacatcatcgaggagtacggcaagggccgcatcttccagtcccgcatccgcgccggcaagtgcaacaccaagtgccagacctccgtgggcggcatcaacaccaacaagaccttccagaacatcgagcgcaacgccctgggcgactgccccaagtacatcaagtccggccagctgaagctggccaccggcctgcgcaacgtgcccgccatcgcctcccgcggcctgttcggcgccatcgccggcttcatcgagggcggctggcccggcctgatcaacggctggtacggcttccagcaccagaacgagcagggcgtgggcatcgccgccgacaaggagtccacccagaaggccatcgaccagatcaccaccaagatcaacaacatcatcgagaagatgaacggcaactacgactccatccgcggcgagttcaaccaggtggagaaccgcatcaacatgctggccgaccgcatcgacgacgccgtgaccgacgtgtggtcctacaacgccaagctgctggtgctgctggagaacgacaagaccctggacatgcacgacgccaacgtgcgcaacctgcacgaccaggtgcgccgcgccctgaagaccaacgccatcgacgagggcaacggctgcttcgagctgctgcacaagtgcaacgactcctgcatggagaccatccgcaacggcacctacaaccacaccgagtacgaggaggagtccaagctgaagcgccaggagatcgagggcatcaagctgaagtccgaggacggcgtgtacaaggccctgtccatctactcctgcatcgcctcctccgtggtgctggtgggcctgatcctggccttcatcatgtgggcctgcaactccggcaactgccgcttcaacatctgcatctaa

SEQ ID NO: 13 - DNA, 1725 bp, synthetic construct
  source 1..1725: mol_type = other DNA
atggccatctccggcgtgcccgtgctgggcttcttcatcatcgccgtgctgatgtccgcccaggagtcctgggccgacgagatctgcatcggctacctgtccaacaactccaccgacaaggtggacaccatcatcgagaacaacgtgaccgtgacctcctccgtggagctggtggagaccgagcacaccggctccttctgctccatcaacggcaagcagcccatctccctgggcgactgctccttcgccggctggatcctgggcaaccccatgtgcgacgacctgatcggcaagacctcctggtcctacatcgtggagaagcccaaccccaccaacggcatctgctaccccggcaccctggaggacgaggaggagctgcgcctgaagttctccggcgtgctggagttctccaagttcgaggccttcacctccaacggctggggcgccgtgaactccggcgccggcgtgaccgccgcctgcaagttcggctcctccaactccttcttccgcaacatggtgtggctgatccaccagtccggcacctaccccgtgatcaagcgcaccttcaacaacaccaagggccgcgacgtgctgatcgtgtggggcatccaccaccccgccaccctgaaggagcaccaggacctgtacaagaaggactcctcctacgtggccgtgggctccgagacctacaaccgccgcttcacccccgagatctccacccgccccaaggtgaacggccaggccggccgcatgaccttctactggaccatggtgaagcccggcgagtccatcaccttcgagtccaacggcgccttcctggccccccgctacgccttcgagatcgtgtccgtgggcaacggcaagctgttccgctccgagctgtccatcgagtcctgctccaccaagtgccagaccgaggtgggcggcatcaacaccaacaagtccttccactccgtgcaccgcaacaccatcggcgactgccccaagtacgtgaacgtgaagtccctgaagctggccaccggcctgcgcaacgtgcccgccatcgcctcccgcggcctgttcggcgccatcgccggcttcatcgagggcggctggcccggcctgatcaacggctggtacggcttccagcaccgcaacgaggagggcaccggcatcgccgccgaccgcgagtccacccagaaggccgtggaccagatcacctccaaggtgaacaacatcgtggaccgcatgaacaccaacttcgagtccgtgcagcacgagttctccgagatcgaggagcgcatcaaccagctgtccaagcacgtggacgactccgtggtggacatctggtcctacaacgcccagctgctggtgtggctggagaacgagaagaccctggacctgcacgactccaacgtgcgcaacctgcacgagaaggtgcgccgcatgctgaaggacaacgccaaggacgagggcaacggctgcttcaccttctaccacaagtgcgacaacgagtgcatcgagaaggtgcgcaacggcacctacgaccacaaggagttcgaggaggagtcccgcatcaaccgccaggagatcgagggcgcccgcctggactcctccggcaacgtgtacaagatcctgtccatctgctcctgcatcgcctcctccctggtgctggccgccctgatcatgggcttcatcctgtgggcctgctccaacggctcctgccgctgcaccatctgcatctaa

SEQ ID NO: 14 - DNA, 1728 bp, synthetic construct
  source 1..1728: mol_type = other DNA
atggccatctccggcgtgcccgtgctgggcttcttcatcatcgccgtgctgatgtccgcccaggagtcctgggcccagaagatccccggcaacgacaactccaccgccaccctgtgcctgggccaccacgccgtgcccaacggcaccatcgtgaagaccatcaccaacgaccgcatcgaggtgaccaacgccaccgagctggtgcagaactcctccatcggcgagatctgcgactccccccaccagatcctggacggcgagaactgcaccctgatcgacgccctgctgggcgacccccagtgcgacggcttccagaacaagaagtgggacctgttcgtggagcggtccaaggcctactccaactgctacccctacgacgtgcccgactacgcctccctgcgctccctggtggcctcctccggcaccctggagttcaagaacgagtccttcaactggaccggcgtgacccagaacggcacctcctccgcctgcatccgcggctcctcctcctccttcttctcccgcctgaactggctgacccacctgaactacacctaccccgccctgaacgtgaccatgcccaacaaggagcagttcgacaagctgtacatctggggcgtgcaccaccccggcaccgacaaggaccagatcttcctgtacgcccagtcctccggccgcatcaccgtgtccaccaagcggtcccagcaggccgtgatccccaacatcggctcccgcccccgcatccgcgacatcccctcccgcatctccatctactggaccatcgtgaagcccggcgacatcctgctgatcaactccaccggcaacctgatcgccccccgcggctacttcaagatccgctccggcaagtcctccatcatgcgctccgacgcccccatcggcaagtgcaagtccgagtgcatcacccccaacggctccatccccaacgacaagcccttccagaacgtgaaccgcatcacctacggcgcctgcccccgctacgtgaagcactccaccctgaagctggccaccggcatgcgcaacgtgcccgagaagcagacccgcggcatcttcggcgccatcgccggcttcatcgagaacggctgggagggcatggtggacggctggtacggcttccgccaccagaactccgagggccgcggccaggccgccgacctgaagtccacccaggccgccatcgaccagatcaacggcaagctgaaccgcctgatcggcaagaccaacgagaagttccaccagatcgagaaggagttctccgaggtggagggccgcgtgcaggacctggagaagtacgtggaggacaccaagatcgacctgtggtcctacaacgccgagctgctggtggccctggagaaccagcacaccatcgacctgaccgactccgagatgaacaagctgttcgagaagaccaagaagcagctgcgcgagaacgccgaggacatgggcaacggctgcttcaagatctaccacaagtgcgacaacgcctgcatcgagtccatccgcaacgagacctacgaccacaacgtgtaccgcgacgaggccctgaacaaccgcttccagatcaagggcgtggagctgaagtccggctacaaggactggatcctgtggatctccttcgccatctcctgcttcctgctgtgcgtggccctgctgggcttcatcatgtgggcctgccagaagggcaacatccgctgcaacatctgcatctaa

SEQ ID NO: 15 - DNA, 1731 bp, synthetic construct
  source 1..1731: mol_type = other DNA
atggccatctccggcgtgcccgtgctgggcttcttcatcatcgccgtgctgatgtccgcccaggagtcctgggccgacaagatctgcatcggctaccacgccaacaactccaccacccaggtggacaccctgctggagaagaacgtgaccgtgacccactccgtggagctgctggagaaccagaaggagaagcggttctgcaagatcatgaacaaggcccccctggacctgaaggactgcaccatcgagggctggatcctgggcaaccccaagtgcgacctgctgctgggcgaccagtcctggtcctacatcgtggagcgccccaacgcccagaacggcatctgctaccccggcgtgctgaacgagctggaggagctgaaggccttcatcggctccggcgagcgcgtggagcggttcgagatgttccccaagtccacctgggccggcgtggacacctcccgcggcgtgaccaacgcctgcccctcctacaccatcgactcctccttctaccgcaacctggtgtggatcgtgaagaccgactccgccacctaccccgtgatcaagggcacctacaacaacaccggcacccagcccatcctgtacttctggggcgtgcaccaccccctggacaccaccgtgcaggacaacctgtacggctccggcgacaagtacgtgcgcatgggcaccgagtccatgaacttcgccaagtcccccgagatcgccgcccgccccgccgtgaacggccagcggtcccgcatcgactactactggtccgtgctgcgccccggcgagaccctgaacgtggagtccaacggcaacctgatcgccccctggtacgcctacaagttcgtgtccaccaacaagaagggcgccgtgttcaagtccgacctgcccatcgagaactgcgacgccacctgccagaccatcaccggcgtgctgcgcaccaacaagaccttccagaacgtgtcccccctgtggatcggcgagtgccccaagtacgtgaagtccgagtccctgcgcctggccaccggcctgcgcaacgtgccccagatcgccacccgcggcatcttcggcgccatcgccggcttcatcgagggcggctggaccggcatgatcgacggctggtacggctaccaccacgagaactcccagggctccggctacgccgccgaccgcgagtccacccagaaggccatcgacggcatcaccaacaaggtgaactccatcatcaacaagatgaacacccagttcgaggccgtggaccacgagttctccaacctggagcgccgcatcggcaacctgaacaagcgcatggaggacggcttcctggacgtgtggacctacaacgccgagctgctggtgctgctggagaacgagcgcaccctggacctgcacgacgccaacgtgaagaacctgtacgagaaggtgaagtcccagctgcgcgacaacgccaacgacctgggcaacggctgcttcgagttctggcacaagtgcgacaacgagtgcatggagtccgtgaagaacggcacctacgactaccccaagtaccagaaggagtccaagctgaaccgccagggcatcgagtccgtgaagctggagaacctgggcgtgtaccagatcctggccatctactccaccgtgtcctcctccctggtgctggtgggcctgatcatggccatcggcctgtggatgtgctccaacggctccatgcagtgccgcatctgcatctaa

SEQ ID NO: 16 - DNA, 1716 bp, synthetic construct
  source 1..1716: mol_type = other DNA
atggccatctccggcgtgcccgtgctgggcttcttcatcatcgccgtgctgatgtccgcccaggagtcctgggccgaccgcatctgcatcggctaccaggccaaccagaacaaccagaccgtgaacaccctgctggagcagaacgtgcccgtgaccggcgcccaggagatcctggagaccaaccacaacggcaagctgtgctccctgaacggcgtgccccccctggacctgcagtcctgcaccctggccggctggctgctgggcaaccccaactgcgacaacctgctggaggccgaggagtggtcctacatcaagatcaacgagaacgcccccgacgacctgtgcttccccggcaacttcgagaacctgcaggacctgctgctggagatgtccggcgtgcagaacttcaccaaggtgaagctgttcaacccccagtccatgaccggcgtgaccaccaacaacgtggaccagacctgccccttcgagggcaagccctccttctaccgcaacctgaactggatccagggcaactccggcctgcccttcaacatcgagatcaagaaccccacctccaaccccctgctgctgctgtggggcatccacaacaccaaggacgccgcccagcagcgcaacctgtacggcaacgactactcctacaccatcttcaacttcggcgagaagtccgaggagttccgccccgacatcggccagcgcgacgagatcaaggcccaccaggaccgcatcgactactactggggctccctgcccgcccagtccaccctgcgcatcgagtccaccggcaacctgatcgcccccgagtacggcttctactacaagcgcaaggagggcaagggcggcctgatgaagtccaagctgcccatctccgactgctccaccaagtgccagacccccctgggcgccctgaactccaccctgcccttccagaacgtgcaccagcagaccatcggcaactgccccaagtacgtgaaggccacctccctgatgctggccaccggcctgcgcaacaacccccagatggagggccgcggcctgttcggcgccatcgccggcttcatcgagggcggctggcagggcatgatcgacggctggtacggctaccaccacgagaaccaggagggctccggctacgccgccgacaaggaggccacccagaaggccgtggacgccatcaccaacaaggtgaactccatcatcgacaagatgaactcccagttcgagtccaacatcaaggagttcaaccgcctggagctgcgcatccagcacctgtccgaccgcgtggacgacgccctgctggacatctggtcctacaacaccgagctgctggtgctgctggagaacgagcgcaccctggacttccacgacgccaacgtgaagaacctgttcgagaaggtgaaggcccagctgaaggacaacgccatcgacgagggcaacggctgcttcctgctgctgcacaagtgcaacaactcctgcatggacgacatcaagaacggcacctacaagtacatggactaccgcgaggagtcccacatcgagaagcagaagatcgacggcgtgaagctgaccgactactcccgctactacaccatgaccctgtactccaccatcgcctcctccgtggtgctgggctccctgatcatcgccgccttcctgtggggctgccagaagggctccatccagtgcaagatctgcatctaa

SEQ ID NO: 17 - DNA, 1782 bp, synthetic construct
  source 1..1782: mol_type = other DNA
atggccatctccggcgtgcccgtgctgggcttcttcatcatcgccgtgctgatgtccgcccaggagtcctgggccgaccgcatctgcaccggcatcacctcctccaactccccccacgtggtgaagaccgccacccagggcgaggtgaacgtgaccggcgtgatccccctgaccaccacccccaccaagtcccacttcgccaacctgaagggcaccgagacccgcggcaagctgtgccccaagtgcctgaactgcaccgacctggacgtggccctgggccgccccaagtgcaccggcaagatcccctccgcccgcgtgtccatcctgcacgaggtgcgccccgtgacctccggctgcttccccatcatgcacgaccgcaccaagatccgccagctgcccaacctgctgcgcggctacgagcacgtgcgcctgtccacccacaacgtgatcaacgccgagggcgcccccggcggcccctacaagatcggcacctccggctcctgccccaacatcaccaacggcaacggcttcttcgccaccatggcctgggccgtgcccgacaagaacaagaccgccaccaaccccctgaccatcgaggtgccctacgtgtgcaccgagggcgaggaccagatcaccgtgtggggcttccactccgacaacgagacccagatggccaagctgtacggcgactccaagccccagaagttcacctcctccgccaacggcgtgaccacccactacgtgtcccagatcggcggcttccccaaccagaccgaggacggcggcctgccccagtccggccgcatcgtggtggactacatggtgcagaagtccggcaagaccggcaccatcacctaccagcgcggcatcctgctgccccagaaggtgtggtgcgcctccggccgctccaaggtgatcaagggctccctgcccctgatcggcgaggccgactgcctgcacgagaagtacggcggcctgaacaagtccaagccctactacaccggcgagcacgccaaggccatcggcaactgccccatctgggtgaagacccccctgaagctggccaacggcaccaagtaccgcccccccgccaagctgctgaaggagcgcggcttcttcggcgccatcgccggcttcctggagggcggctgggagggcatgatcgccggctggcacggctacacctcccacggcgcccacggcgtggccgtggccgccgacctgaagtccacccaggaggccatcaacaagatcaccaagaacctgaactccctgtccgagctggaggtgaagaacctgcagcgcctgtccggcgccatggacgagctgcacaacgagatcctggagctggacgagaaggtggacgacctgcgcgccgacaccatctcctcccagatcgagctggccgtgctgctgtccaacgagggcatcatcaactccgaggacgagcacctgctggccctggagcgcaagctgaagaagatgctgggcccctccgccgtggagatcggcaacggctgcttcgagaccaagcacaagtgcaaccagacctgcctggacaagatcgccgccggcaccttcgacgccggcgagttctccctgcccaccttcgactccctgaacatcaccgccgcctccctgaacgacgacggcctggacaaccacaccatcctgctgtactactccaccgccgcctcctccctggccgtgaccctgatgatcgccatcttcgtggtgtacatggtgtcccgcgacaacgtgtcctgctccatctgcctgtaa

SEQ ID NO: 18 - DNA, 1785 bp, synthetic construct
  source 1..1785: mol_type = other DNA
atggccatctccggcgtgcccgtgctgggcttcttcatcatcgccgtgctgatgtccgcccaggagtcctgggccgaccgcatctgcaccggcatcacctcctccaactccccccacgtggtgaagaccgccacccagggcgaggtgaacgtgaccggcgtgatccccctgaccaccacccccaccaagtcctacttcgccaacctgaagggcacccgcacccgcggcaagctgtgccccgactgcctgaactgcaccgacctggacgtggccctgggccgccccatgtgcgtgggcaccaccccctccgccaaggcctccatcctgcacgaggtgcgccccgtgacctccggctgcttccccatcatgcacgaccgcaccaagatccgccagctgcccaacctgctgcgcggctacgagaagatccgcctgtccacccagaacgtgatcgacgccgagaaggcccccggcggcccctaccgcctgggcacctccggctcctgccccaacgccacctccaagatcggcttcttcgccaccatggcctgggccgtgcccaaggacaactacaagaacgccaccaaccccctgaccgtggaggtgccctacatctgcaccgagggcgaggaccagatcaccgtgtggggcttccactccgacaacaagacccagatgaagtccctgtacggcgactccaacccccagaagttcacctcctccgccaacggcgtgaccacccactacgtgtcccagatcggcgacttccccgaccagaccgaggacggcggcctgccccagtccggccgcatcgtggtggactacatgatgcagaagcccggcaagaccggcaccatcgtgtaccagcgcggcgtgctgctgccccagaaggtgtggtgcgcctccggccgctccaaggtgatcaagggctccctgcccctgatcggcgaggccgactgcctgcacgaggagtacggcggcctgaacaagtccaagccctactacaccggcaagcacgccaaggccatcggcaactgccccatctgggtgaagacccccctgaagctggccaacggcaccaagtaccgcccccccgccaagctgctgaaggagcgcggcttcttcggcgccatcgccggcttcctggagggcggctgggagggcatgatcgccggctggcacggctacacctcccacggcgcccacggcgtggccgtggccgccgacctgaagtccacccaggaggccatcaacaagatcaccaagaacctgaactccctgtccgagctggaggtgaagaacctgcagcgcctgtccggcgccatggacgagctgcacaacgagatcctggagctggacgagaaggtggacgacctgcgcgccgacaccatctcctcccagatcgagctggccgtgctgctgtccaacgagggcatcatcaactccgaggacgagcacctgctggccctggagcgcaagctgaagaagatgctgggcccctccgccgtggacatcggcaacggctgcttcgagaccaagcacaagtgcaaccagacctgcctggaccgcatcgccgccggcaccttcaacgccggcgagttctccctgcccaccttcgactccctgaacatcaccgccgcctccctgaacgacgacggcctggacaaccacaccatcctgctgtactactccaccgccgcctcctccctggccgtgaccctgatgctggccatcttcatcgtgtacatggtgtcccgcgacaacgtgtcctgctccatctgcctgtaa

SEQ ID NO: 19 - DNA, 1707 bp, synthetic construct
  source 1..1707: mol_type = other DNA
atggagaagatcgtgctgctgttcgccatcgtgtccctggtgaagtccgaccagatctgcatcggctaccacgccaacaactccaccgagcaggtggacaccatcatggagaagaacgtgaccgtgacccacgcccaggacatcctggagaagaagcacaacggcaagctgtgcgacctggacggcgtgaagcccctgatcctgcgcgactgctccgtggccggctggctgctgggcaaccccatgtgcgacgagttcatcaacgtgcccgagtggtcctacatcgtggagaaggccaaccccgtgaacgacctgtgctaccccggcgacttcaacgactacgaggagctgaagcacctgctgtcccgcatcaaccacttcgagaagatccagatcatccccaagtcctcctggtcctcccacgaggcctccctgggcgtgtcctccgcctgcccctaccagggcaagtcctccttcttccgcaacgtggtgtggctgatcaagaagaactccacctaccccaccatcaaacgctcctacaacaacaccaaccaggaggacctgctggtgctgtggggcatccaccaccccaacgacgccgccgagcagaccaagctgtaccagaaccccaccacctacatctccgtgggcacctccaccctgaaccagcgcctggtgccccgcatcgccacccgctccaaggtgaacggccagtccggccgcatggagttcttctggaccatcctgaagcccaacgacgccatcaacttcgagtccaacggcaacttcatcgcccccgagtacgcctacaagatcgtgaagaagggcgactccaccatcatgaagtccgagctggagtacggcaactgcaacaccaagtgccagacccccatgggcgccatcaactcctccatgcccttccacaacatccaccccctgaccatcggcgagtgccccaagtacgtgaagtccaaccgcctggtgctggccaccggcctgcgcaactccccccagcgcgagcgccgccgcaagaagcgcggcctgttcggcgccatcgccggcttcatcgagggcggctggcagggcatggtggacggctggtacggctaccaccactccaacgagcagggctccggctacgccgccgacaaggagtccacccagaaggccatcgacggcgtgaccaacaaggtgaactccatcatcgacaagatgaacacccagttcgaggccgtgggccgcgagttcaacaacctggagcgccgcatcgagaacctgaacaagaagatggaggacggcttcctggacgtgtggacctacaacgccgagctgctggtgctgatggagaacgagcgcaccctggacttccacgactccaacgtgaagaacctgtacgacaaggtgcgcctgcagctgcgcgacaacgccaaggagctgggcaacggctgcttcgagttctaccacaagtgcgacaacgagtgcatggagtccgtgcgcaacggcacctacgactacccccagtactccgaggaggcccgcctgaagcgcgaggagatctccggcgtgaagctggagtccatcggcatctaccagatcctgtccatctactccaccgtggcctcctccctggccctggccatcatggtggccggcctgtccctgtggatgtgctccaacggctccctgcagtgccgcatctgcatctaa

SEQ ID NO: 20 - DNA, 1683 bp, synthetic construct
  source 1..1683: mol_type = other DNA
atgaacacccagatcctggtgttcgccctgatcgccatcatccccaccaacgccgacaagatctgcctgggccaccacgccgtgtccaacggcaccaaggtgaacaccctgaccgagcgcggcgtggaggtggtgaacgccaccgagaccgtggagcgcaccaacatcccccgcatctgctccaagggcaagcgcaccgtggacctgggccagtgcggcctgctgggcaccatcaccggccccccccagtgcgaccagttcctggagttctccgccgacctgatcatcgagcgccgcgagggctccgacgtgtgctaccccggcaagttcgtgaacgaggaggccctgcgccagatcctgcgcgagtccggcggcatcgacaaggaggccatgggcttcacctactccggcatccgcaccaacggcgccacctccgcctgccgccgctccggctcctccttctacgccgagatgaagtggctgctgtccaacaccgacaacgccgccttcccccagatgaccaagtcctacaagaacacccgcaagtcccccgccctgatcgtgtggggcatccaccactccgtgtccaccgccgagcagaccaagctgtacggctccggcaacaagctggtgaccgtgggctcctccaactaccagcagtccttcgtgccctcccccggcgcccgcccccaggtgaacggcctgtccggccgcatcgacttccactggctgatgctgaaccccaacgacaccgtgaccttctccttcaacggcgccttcatcgcccccgaccgcgcctccttcctgcgcggcaagtccatgggcatccagtccggcgtgcaggtggacgccaactgcgagggcgactgctaccactccggcggcaccatcatctccaacctgcccttccagaacatcgactcccgcgccgtgggcaagtgcccccgctacgtgaagcagcggtccctgctgctggccaccggcatgaagaacgtgcccgagatccccaagggccgcggcctgttcggcgccatcgccggcttcatcgagaacggctgggagggcctgatcgacggctggtacggcttccgccaccagaacgcccagggcgagggcaccgccgccgactacaagtccacccagtccgccatcgaccagatcaccggcaagctgaaccgcctgatcgagaagaccaaccagcagttcgagctgatcgacaacgagttcaacgaggtggagaagcagatcggcaacgtgatcaactggacccgcgactccatcaccgaggtgtggtcctacaacgccgagctgctggtggccatggagaaccagcacaccatcgacctggccgactccgagatggacaagctgtacgagcgcgtgaagcgccagctgcgcgagaacgccgaggaggacggcaccggctgcttcgagatcttccacaagtgcgacgacgactgcatggcctccatccgcaacaacacctacgaccactccaagtaccgcgaggaggccatgcagaaccgcatccagatcgaccccgtgaagctgtcctccggctacaaggacgtgatcctgtggttctccttcggcgcctcctgcttcatcctgctggccatcgtgatgggcctggtgttcatctgcgtgaagaacggcaacatgcgctgcaccatctgcatctaa

(19) United States
(12) Patent Application Publication
(10) Pub. No.: US 2024/0374708 A1
(43) Pub. Date: Nov. 14, 2024

(54) Universal Influenza Vaccine and Methods of Use

(71) Applicant: The Trustees of the University of Pennsylvania, Philadelphia, PA (US)

(72) Inventors: Scott Hensley, Philadelphia, PA (US); Drew Weissman, Wynnewood, PA (US)

(21) Appl. No.: 18/689,656

(22) PCT Filed: Sep. 7, 2022
(86) PCT No.: PCT/US2022/076013

§ 371 Date: Mar. 6, 2024

Related U.S. Application Data
(60) Provisional application No. 63/241,281, filed on Sep. 7, 2021.

(51) Int. Cl. A61K 39/145; A61K 9/127; A61K 9/51; A61K 39/00; A61P 31/16; C12N 7/00

(52) U.S. Cl. CPC A61K 39/145; A61K 9/127; A61K 9/5123; A61P 31/16; C12N 7/00; A61K 2039/53; A61K 2039/545; A61K 2039/55555; A61K 2039/575; A61K 2039/70; C12N 2760/16133; C12N 2760/16234

(57) ABSTRACT

Provided is a twenty-hemagglutinin antigen (HA) universal influenza vaccine comprising HA antigens from each known influenza A and influenza B lineage and methods of use thereof to treat or prevent influenza.

### CROSS REFERENCE TO RELATED APPLICATIONS

This application claims priority to U.S. Provisional Patent Application No. 63/241,281, filed Sep. 7, 2021 which is hereby incorporated by reference herein in its entirety.

### STATEMENT REGARDING FEDERALLY SPONSORED RESEARCH OR DEVELOPMENT

This invention was made with government support under AI150677 and AI108686 awarded by National Institutes of Health. The government has certain rights in the invention.

### BACKGROUND OF THE INVENTION

There are at least 18 different influenza A virus subtypes that circulate in animal reservoirs and occasionally these viruses enter the human population and cause a pandemic (Krammer et al., 2018, Nat Rev Dis Primers, 4:3). H1N1, H2N2, and H3N2 influenza A viruses (IAVs) have caused pandemics over the last century. Currently, H1N1, H3N2, and 2 antigenically distinct lineages of influenza B viruses (IBVs) circulate seasonally in the human population. Although surveillance programs and modeling studies have provided increased knowledge of pandemic risk (Nelson et al., 2019, Epidemics, 26:116-127; Harrington et al., 2021, Exp Mol Med, 53:737-749), which influenza subtype will cause the next pandemic cannot accurately be predicted. Several ‘universal influenza vaccines’ are in development to provide protection against diverse influenza virus subtypes (Erbelding et al, 2018, J Infect Dis, 218:347-354). However, most ‘universal influenza vaccines’ include only a limited number of antigens that possess epitopes that are conserved across different influenza virus subtypes (Yassine et al., 2015, Nat Med 21, 1065-1070; Corbett et al., 2019, mBio, 10; Nachbagauer et al., 2021, Nat Med, 27:106-114).

Thus, there is a need in the art for improved universal influenza vaccines. The present invention addresses this need.

### SUMMARY OF THE INVENTION

In one embodiment, the invention relates to a composition for inducing an immune response against one or more influenza viruses in a subject, the composition comprising a combination of at least two lipid nanoparticle (LNPs) comprising a combination of nucleoside-modified RNA molecules encoding at least two influenza virus antigens, wherein the combination of at least two nucleoside-modified RNA molecules encode hemagglutinin (HA) antigens, or fragments thereof, are from at least two of influenza A virus H1, H2, H3, H4, H5, H6, H7, H8, H9, H10, H11, H12, H13, H14, H15, H16, H17, and H18, and influenza B virus Vic and Yam.

In one embodiment, the HA antigen, or fragment thereof, is a full length HA antigen or a fragment thereof, HA-stalk domain or a fragment thereof, HA-head domain or a fragment thereof, or any combination thereof.

In one embodiment, the composition comprises nucleoside-modified RNA molecules encoding HA antigens from each of influenza A virus H1, H2, H3, H4, H5, H6, H7, H8, H9, H10, H11, H12, H13, H14, H15, H16, H17, and H18, and influenza B virus Vic and Yam.

In one embodiment, the composition comprises at least one additional viral antigen.

In one embodiment, the composition comprises a combination of at least two mRNA molecules encoded by SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 or SEQ ID NO:20.

In one embodiment, the composition comprises an adjuvant.

In one embodiment, the nucleoside-modified RNA molecules are encapsulated within the LNPs.

In one embodiment, the nucleoside-modified RNA molecules comprise at least one pseudouridine, 1-methyl pseudouridine, or 5-methyl-uridine.

In one embodiment, the composition is a universal influenza vaccine.

In one embodiment, the invention relates to a method of inducing an immune response against multiple strains of influenza virus in a subject comprising administering to the subject an effective amount of a composition comprising a combination of at least two lipid nanoparticle (LNPs) wherein each LNP comprises a nucleoside-modified RNA encoding at least one influenza virus antigen or a fragment thereof, and further wherein the combination of at least two LNPs together comprise at least two nucleoside-modified RNA molecule encoding hemagglutinin (HA) antigens, or fragments thereof selected from the group consisting of influenza A virus H1, H2, H3, H4, H5, H6, H7, H8, H9, H10, H11, H12, H13, H14, H15, H16, H17, and H18, and influenza B virus Vic and Yam.

In one embodiment, the HA antigen, or fragment thereof, is a full length HA antigen or a fragment thereof, HA-stalk domain or a fragment thereof, HA-head domain or a fragment thereof, and any combination thereof.

In one embodiment, the composition comprises nucleoside-modified RNA molecules encoding HA antigens from each of influenza A virus H1, H2, H3, H4, H5, H6, H7, H8, H9, H10, H11, H12, H13, H14, H15, H16, H17, and H18, and influenza B virus Vic and Yam.

In one embodiment, the composition comprises at least one additional viral antigen.

In one embodiment, the composition comprises a combination of mRNA molecules encoded by nucleotide sequences selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

In one embodiment, the composition comprises an adjuvant.

In one embodiment, the nucleoside-modified RNA molecules are encapsulated within the LNPs.

In one embodiment, the nucleoside-modified RNA molecules comprise at least one pseudouridine, 1-methyl pseudouridine, or 5-methyl-uridine.

In one embodiment, the composition is administered by an intradermal, subcutaneous, inhalation, intranasal, or intramuscular delivery route.

In one embodiment, the composition is administered a single time.

In one embodiment, the composition is administered multiple times.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts exemplary experimental data demonstrating that all HA mRNA-LNPs are immunogenic when delivered to mice individually. Mice were vaccinated intramuscularly with 3 μg of single HA mRNA-LNPs. Sera were collected 28 days post-vaccination and antibody levels were quantified by ELISA. HA are arranged based on protein sequence homology. HA protein sequences were aligned with MUSCLE (v.3.8.425) within Geneious (v.11.1.4). The alignment was used to construct a consensus neighbor-joining tree (Jukes-Cantor method) within Geneious. Group 1 HAs are colored blue and group 2 HAs are colored red. 3 animals were included per experimental group. Data are shown as mean.

FIG. 2A through FIG. 2F depict exemplary experimental data demonstrating that the 20 HA mRNA-LNP vaccine elicits long-lived antibody responses that react to all 20 HAs. FIG. 2A depicts data demonstrating that mice were vaccinated i.m. simultaneously with 20 different HA mRNA-LNPs. For this, mice were vaccinated with a combined dose of 50 μg of HA mRNA-LNPs (2.5 μg of each individual HA mRNA-LNP). Other groups of mice were vaccinated i.m. with 50 μg of H1 mRNA-LNP (FIG. 2B), 50 μg of H3 mRNA-LNP (FIG. 2C), 50 μg of IBV HA mRNA-LNP (FIG. 2D) or PBS (FIG. 2E). Sera were isolated 28 days (FIG. 2A-FIG. 2E) or 118 days (FIG. 2F) later and antibody reactivity to different HAs were quantified using ELISAs coated with recombinant proteins. 8 mice were included for each experimental group. Group 1 HAs are shown in blue and group 2 HAs are shown in red. AUC; area under the curve. Data are representative of 2 independent experiments and are shown as mean+/−SEM.

FIG. 3A through FIG. 3H depict that the 20 HA mRNA-LNP vaccine elicits diverse antibodies that can target different epitopes. Serum samples were collected from mice 28 days after (FIG. 3A) H1, (FIG. 3B) H3, (FIG. 3C) IBV, or (FIG. 3D) 20 HA mRNA-LNP vaccination. Samples were absorbed with magnetic beads coupled to recombinant H1, H3, or no HA (mock), and antibody levels remaining in the unabsorbed fraction were quantified by ELISA (FIG. 3A-FIG. 3D). Hemagglutination-inhibition (HAI) assays were completed using virus with (FIG. 3E) A/Michigan/45/2015 H1 or (FIG. 3F) A/Singapore/INFIMH-16-0019/2016 H3. HA stalk-reactive antibodies were quantified by ELISAs coated with ‘headless’ (FIG. 3G) H1 and (FIG. 3H) H3 recombinant proteins. AUC; area under the curve. (FIG. 3A-FIG. 3F) 6 mice were included for each experimental group. (FIG. 3G-FIG. 3H) 12 mice were included for each experimental group. Data are representative of 2-3 independent experiments and are shown as mean+/−SEM. Antibody titers were compared using a one-way ANOVA with Tukey's post-test. *p<0.05.

FIG. 4A through FIG. 4D depict exemplary experimental data demonstrating that the 20 HA mRNA-LNP vaccine protects mice from challenge with an antigenically distinct H1N1 strain. Mice were vaccinated with mRNA-LNPs encoding H1 (blue), H3 (red), IBV (gray), or 20 HAs (purple) and then 4 months later they were infected i.n. with a 500 tissue culture infectious dose (TCID)50 of A/Puerto Rico/8/1934 H1N1 influenza virus. (FIG. 4A) Weight loss, (FIG. 4B) clinical scores, and (FIG. 4C) survival were monitored for 14 days following infection. (FIG. 4D) Sera from mice was collected 28 days after vaccination with the 20 HA mRNA-LNP vaccine and 800 μl of serum was passively transferred into naïve mice. As a control, naïve serum was passively transferred into naïve mice and PBS into naïve mice. Mice were then were infected i.n. with a 500 tissue culture infectious dose (TCID)50 of A/Puerto Rico/8/1934 H1N1 influenza virus and survival was monitored for 14 days. 12-15 mice were included per immunization group. Data are representative of two independent experiments and are shown as mean+/−SEM.

FIG. 5A through FIG. 5D depict exemplary experimental data demonstrating that the 20 HA mRNA-LNP vaccine elicits antibodies that react to the antigenically distinct A/Puerto Rico/8/1934 H A. Mice were immunized with mRNA-LNPs encoding H1 (blue), H3 (red), IBV (gray), or 20 HA (purple) and sera were collected 118 days post vaccination. Antibodies reactive to the A/Puerto Rico/8/1934 full length HA (FIG. 5A), HA head (FIG. 5B), and HA stalk (FIG. 5C) were quantified by ELISA. (FIG. 5D) HAI assays using A/Puerto Rico/8/1934 were also completed and all samples were negative. 9 animals were included per experimental group. Data are shown as mean+/−SEM.

FIG. 6A through FIG. 6E depict exemplary experimental data demonstrating that the 20 HA mRNA-LNP vaccine protects ferrets from challenge with an antigenically distinct H1N1 strain. Ferrets were primed with 60 μg of the 20 HA mRNA-LNP vaccine (3 μg of each HA mRNA-LNP) and then boosted with the same vaccine dose 28 days later. (FIG. 6A) Sera were collected 28 after the first and second vaccinations and antibody reactivity to different HAs were quantified using ELISAs coated with recombinant proteins. 28 days after the second vaccination, ferrets were infected i.n. with a 10⁶ tissue culture infectious dose (TCID)₅₀ of A/Ruddy turnstone/Delaware/300/2009 H1N1 influenza virus. As a control, unvaccinated animals were also infected with the virus. (FIG. 6B) Weight loss (FIG. 6C) survival and (FIG. 6D) symptoms were monitored for 14 days following infection. (FIG. 6E) Virus levels in nasal wash samples isolated 1-7 days following infection were quantified using (TCID)₅₀ assays. 4 ferrets were included for each experimental group. Data shown are mean+/−SEM (FIG. 6A, FIG. 6B, FIG. 6E). Weight loss and nasal wash titers were compared using a t-test at each day (FIG. 6B, FIG. 6E).

### DETAILED DESCRIPTION

The present invention relates to compositions and methods for inducing an immune response against influenza virus in a subject. In some embodiments, the invention provides a composition comprising a combination of lipid nanoparticles (LNPs) comprising nucleoside-modified RNA molecules encoding a combination of at least twenty influenza virus HA antigens from each of influenza A virus H1, H2, H3, H4, H5, H6, H7, H8, H9, H10, H11, H12, H13, H14, H15, H16, H17, and H18, and influenza B virus Vic and Yam. In some embodiments, the composition further comprises at least 1, 2, 3, 4, 5, 6, 7, 8, 9, 10, 11, 12, 13, 14, 15, 16, 17, 18, 19, 20, or more than twenty additional viral antigens.

In one embodiment, the composition is a vaccine comprising a combination of at least twenty LNPs, wherein each LNP comprises a nucleoside-modified RNA molecule encoding an HA antigen from influenza A virus H1, H2, H3, H4, H5, H6, H7, H8, H9, H10, H11, H12, H13, H14, H15, H16, H17, and H18, and influenza B virus Vic and Yam, wherein the vaccine induces an immune response in the subject to multiple influenza virus strains, and therefore the vaccine is a universal influenza vaccine.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20%, ±10%, ±5%, ±1%, or ±0.1% from the specified value, as such variations are appropriate to perform the disclosed methods.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody, which is generated using recombinant DNA technology. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned), synthesizing the RNA, or other technology, which is available and well known in the art.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “immunogen” as used herein, is intended to denote a substance of matter, which is capable of inducing an adaptive immune response in an individual, where said adaptive immune response is capable of inducing an immune response, which significantly engages pathogenic agents, which share immunological features with the immunogen. “Immunogen” refers to any substance introduced into the body in order to generate an immune response. That substance can a physical molecule, such as a protein, or can be encoded by a vector, such as DNA, mRNA, or a virus.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequence or a partial nucleotide sequence encoding a protein that elicits an adaptive immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“Immune response,” as the term is used herein, means a process involving the activation and/or induction of an effector function in, by way of non-limiting examples, a T cell, B cell, natural killer (NK) cell, and/or an antigen-presenting cell (APC). Thus, an immune response, as would be understood by the skilled artisan, includes, but is not limited to, any detectable antigen-specific activation and/or induction of a helper T cell or cytotoxic T cell activity or response, production of antibodies, antigen presenting cell activity or infiltration, macrophage activity or infiltration, neutrophil activity or infiltration, and the like.

As used herein, an “immunogenic composition” may comprise an antigen (e.g., a peptide or polypeptide), a nucleic acid encoding an antigen, a cell expressing or presenting an antigen or cellular component, a virus expressing or presenting an antigen or cellular component, or a combination thereof. In particular embodiments, the composition comprises or encodes all or part of any peptide antigen described herein, or an immunogenically functional equivalent thereof. In other embodiments, the composition is in a mixture that comprises an additional immunostimulatory agent or nucleic acids encoding such an agent. Immunostimulatory agents include but are not limited to an additional antigen, an immunomodulator, an antigen presenting cell, lipid nanoparticle, or an adjuvant. In other embodiments, one or more of the additional agent(s) is covalently bonded to the antigen or an immunostimulatory agent, in any combination.

As used herein, the term “vaccine” refers to a composition that induces an immune response upon inoculation into a subject. In some embodiments, the induced immune response provides protective immunity.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

As used herein, a nucleotide sequence is “substantially homologous” to any of the nucleotide sequences described herein when its nucleotide sequence has a degree of identity with respect to the original nucleotide sequence at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

As used herein, an amino acid sequence is “substantially homologous” to any of the amino acid sequences described herein when its amino acid sequence has a degree of identity with respect to the original amino acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. The identity between two amino acid sequences can be determined by using the BLASTN algorithm (BLAST Manual, Altschul, S., et al., NCBI NLM NIH Bethesda, Md. 20894, Altschul, S., et al., J. Mol. Biol. 215: 403-410 (1990)).

The term “variant” as used herein with respect to a nucleic acid refers (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto. A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof.

The term “variant” as used with respect to a peptide or polypeptide refers to a peptide or polypeptide that differs in amino acid sequence by the insertion, deletion, or conservative substitution of amino acids, but retain at least one biological activity. Variant may also refer to a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., 1982, J. Mol. Biol. 157:105-132). The hydropathic index of an amino acid is based on a consideration of its hydrophobicity and charge. It is known in the art that amino acids of similar hydropathic indexes can be substituted and still retain protein function. In one aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

As used herein, the terms “fragment” or “functional fragment” refer to a fragment of an influenza virus antigen or a nucleic acid sequence encoding an influenza virus antigen that, when administered to a subject, provides an increased immune response. Fragments are generally 10 or more amino acids or nucleic acids in length. “Fragment” may mean a polypeptide fragment of an antigen that is capable of eliciting an immune response in a subject. A fragment of an antigen may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antigen, excluding any heterologous signal peptide added. The fragment may comprise a fragment of a polypeptide that is 95% or more, 96% or more, 97% or more, 98% or more or 99% or more identical to the antigen and additionally comprise an N terminal methionine or heterologous signal peptide which is not included when calculating percent identity.

A fragment of a nucleic acid sequence that encodes an antigen may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antigen and additionally optionally comprise sequence encoding an N terminal methionine or heterologous signal peptide which is not included when calculating percent identity.

“Isolated” means altered or removed from the natural state. For example, a nucleic acid or a peptide naturally present in a living subject is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleosides (nucleobase bound to ribose or deoxyribose sugar via N-glycosidic linkage) are used. “A” refers to adenosine, “C” refers to cytidine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. The phrase nucleotide sequence that encodes a protein or an RNA may also include introns to the extent that the nucleotide sequence encoding the protein may in some version contain an intron(s). In addition, the nucleotide sequence may contain modified nucleosides that are capable of being translated by translational machinery in a cell. Exemplary modified nucleosides are described elsewhere herein. For example, an mRNA where some or all of the uridines have been replaced with pseudouridine, 1-methyl pseudouridine, 5-methyl-uridine or another modified nucleoside, such as those described elsewhere herein. In some embodiments, the nucleotide sequence may contain a sequence where some or all cytodines are replaced with methylated cytidine, or another modified nucleoside, such as those described elsewhere herein.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In some instances, the polynucleotide or nucleic acid of the invention is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27: 196-197).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence. By way of one non-limiting example, a promoter that is recognized by bacteriophage RNA polymerase and is used to generate the mRNA by in vitro transcription.

The term “adjuvant” as used herein is defined as any molecule to enhance an antigen-specific adaptive immune response.

In some embodiments, “pseudouridine” refers to m¹acp³Ψ (1-methyl-3-(3-amino-3-carboxypropyl) pseudouridine). In another embodiment, the term refers to m¹Ψ (1-methylpseudouridine). In another embodiment, the term refers to Ψm (2′-O-methylpseudouridine. In another embodiment, the term refers to m⁵D (5-methyldihydrouridine). In another embodiment, the term refers to m³Ψ (3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present invention.

The term “lipid nanoparticle” refers to a particle having at least one dimension on the order of nanometers (e.g., 1-1,000 nm), which includes one or more lipids.

The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In some embodiments, the cationic lipid comprises a zwitterionic lipid that assumes a positive charge on pH decrease.

The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

The term “anionic lipid” refers to any lipid that is negatively charged at physiological pH.

The term “polymer conjugated lipid” refers to a molecule comprising both a lipid portion and a polymer portion. An example of a polymer conjugated lipid is a pegylated lipid.

The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

“Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

The terms “subject,” “patient,” “individual,” and the like are used interchangeably herein, and refer to any animal, or cells thereof whether in vitro or in situ, amenable to the methods described herein. In some non-limiting embodiments, the patient, subject or individual is a mammal, bird, poultry, cattle, pig, horse, sheep, ferret, primate, dog, cat, guinea pig, rabbit, bat, or human.

A “disease” is a state of health of a subject wherein the subject cannot maintain homeostasis, and wherein if the disease is not ameliorated then the subject's health continues to deteriorate.

In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, such as a human.

To “treat” a disease as the term is used herein, means to reduce the frequency or severity of at least one sign or symptom of a disease or disorder experienced by a subject.

An “effective amount” as used herein, means an amount which provides a therapeutic or prophylactic benefit.

The term “therapeutic” as used herein means a treatment and/or prophylaxis. A therapeutic effect is obtained by suppression, diminution, remission, prevention, or eradication of at least one sign or symptom of a disease or disorder.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to compositions and methods for inducing an immune response against multiple strains of influenza virus in a subject. In some embodiments, the invention provides a composition comprising a combination of lipid nanoparticles comprising nucleoside-modified RNA molecules encoding a combination of at least twenty influenza virus HA antigens from each of influenza A virus H1, H2, H3, H4, H5, H6, H7, H8, H9, H10, H11, H12, H13, H14, H15, H16, H17, and H18, and influenza B virus Vic and Yam. In some embodiments, the composition further comprises at least 1, 2, 3, 4, 5, 6, 7, 8, 9, 10, 11, 12, 13, 14, 15, 16, 17, 18, 19, 20, or more than twenty additional viral antigens. In some embodiments, one or more additional viral antigens may be from human immunodeficiency virus (HIV), Chikungunya virus (CHIKV), dengue fever virus, papilloma viruses, for example, human papillomoa virus (HPV), polio virus, hepatitis viruses, for example, hepatitis A virus (HAV), hepatitis B virus (HBV), hepatitis C virus (HCV), hepatitis D virus (HDV), and hepatitis E virus (HEV), smallpox virus (Variola major and minor), vaccinia virus, rhinoviruses, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, lassa virus, arenavirus, Merkel cell polyoma virus (MCV), influenza virus, a coronavirus including but not limited to severe acute respiratory syndrome-coronavirus 2, a cancer causing virus, or any combination thereof. In one embodiment, one or more additional viral antigen are influenza viral antigens. Exemplary additional influenza virus antigens include, but are not limited to, a full length HA antigen or a fragment thereof, an HA-stalk domain or a fragment thereof, an HA-head domain or a fragment thereof, a full length neuraminidase (NA) antigen or a fragment thereof, a NA-stalk domain or a fragment thereof, NA-head domain or a fragment thereof, full length NP antigen or a fragment thereof, full length matrix protein 1 (M1) antigen or a fragment thereof, full length matrix-2 (M2) ion channel antigen or a fragment thereof, a M2 ion channel-extracellular domain or a fragment thereof, a M2 ion channel-intracellular domain or a fragment thereof, or any combination thereof.

For example, in one embodiment, the composition is a vaccine comprising a combination of lipid nanoparticles comprising nucleoside-modified RNA molecules encoding a combination of at least twenty influenza virus HA antigens from each of influenza A virus H1, H2, H3, H4, H5, H6, H7, H8, H9, H10, H11, H12, H13, H14, H15, H16, H17, and H18, and influenza B virus Vic and Yam, wherein the vaccine induces an immune response in the subject to multiple influenza virus strains, and therefore the vaccine is a universal influenza vaccine. In some embodiments, the at least one nucleoside-modified RNA molecules are encapsulated in one or more LNP. In some embodiments, the composition comprises a combination of at least 2, 3, 4, 5, 6, 7, 8, 9, 10, 11, 12, 13, 14, 15, 16, 17, 18, 19, 20 or more than twenty LNPs comprising nucleoside-modified RNA molecules encoding the combination of at least 20 influenza virus HA antigens from each of influenza A virus H1, H2, H3, H4, H5, H6, H7, H8, H9, H10, H11, H12, H13, H14, H15, H16, H17, and H18, and influenza B virus Vic and Yam. In some embodiments, the composition comprises a combination of at least 20 LNPs comprising nucleoside-modified RNA molecules encoding the combination of at least 20 influenza virus HA antigens from each of influenza A virus H1, H2, H3, H4, H5, H6, H7, H8, H9, H10, H11, H12, H13, H14, H15, H16, H17, and H18, and influenza B virus Vic and Yam.

### Vaccine

In one embodiment, the present invention provides an immunogenic composition for inducing an immune response against influenza virus in a subject. For example, in one embodiment, the immunogenic composition is a vaccine. For a composition to be useful as a vaccine, the composition must induce an immune response against the influenza virus antigen in a cell, tissue or subject. In some embodiments, the composition induces a broad immune response against multiple strains of influenza virus in a cell, tissue or subject. In some instances, the vaccine induces a protective immune response in the subject.

A vaccine of the present invention may vary in its composition of nucleic acid and/or cellular components. In one embodiment, the vaccine comprises a combination of nucleic acid molecules encoding a combination of at least 20 influenza virus HA antigens from each of influenza A virus H1, H2, H3, H4, H5, H6, H7, H8, H9, H10, H11, H12, H13, H14, H15, H16, H17, and H18, and influenza B virus Vic and Yam. In some embodiments, the vaccine further comprises one or more additional nucleic acid molecules encoding at least 1, 2, 3, 4, 5, 6, 7, 8, 9, 10, 11, 12, 13, 14, 15, 16, 17, 18, 19, 20, or more than twenty additional viral antigens. In some embodiments, one or more additional viral antigens may be from human immunodeficiency virus (HIV), Chikungunya virus (CHIKV), dengue fever virus, papilloma viruses, for example, human papillomoa virus (HPV), polio virus, hepatitis viruses, for example, hepatitis A virus (HAV), hepatitis B virus (HBV), hepatitis C virus (HCV), hepatitis D virus (HDV), and hepatitis E virus (HEV), smallpox virus (Variola major and minor), vaccinia virus, rhinoviruses, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, lassa virus, arenavirus, Merkel cell polyoma virus (MCV), influenza virus, a coronavirus including but not limited to severe acute respiratory syndrome-coronavirus 2, a cancer causing virus, or any combination thereof. In one embodiment, one or more additional viral antigen are influenza viral antigens. Exemplary additional influenza virus antigens include, but are not limited to, a full length HA antigen or a fragment thereof, an HA-stalk domain or a fragment thereof, an HA-head domain or a fragment thereof, a full length NA antigen or a fragment thereof, a NA-stalk domain or a fragment thereof, NA-head domain or a fragment thereof, full length NP antigen or a fragment thereof, full length M1 antigen or a fragment thereof, full length M2 ion channel antigen or a fragment thereof, a M2 ion channel-extracellular domain or a fragment thereof, a M2 ion channel-intracellular domain or a fragment thereof, or any combination thereof.

In a non-limiting example, one or more nucleic acid molecule encoding an influenza virus HA antigen might also be formulated with an adjuvant. In another non-limiting example, the LNP vaccine may comprise one or more adjuvants. A vaccine of the present invention, and its various components, may be prepared and/or administered by any method disclosed herein or as would be known to one of ordinary skill in the art, in light of the present disclosure.

In various embodiments, the induction of immunity by the expression of the influenza virus antigens can be detected by observing in vivo or in vitro the response of all or any part of the immune system in the host against one or more influenza virus antigen.

For example, a method for detecting the induction of cytotoxic T lymphocytes is well known. A foreign substance that enters the living body is presented to T cells and B cells by the action of antigen presenting cells (APCs). Some T cells that respond to the antigen presented by APC in an antigen specific manner differentiate into cytotoxic T cells (also referred to as cytotoxic T lymphocytes or CTLs) due to stimulation by the antigen. These antigen-stimulated cells then proliferate. This process is referred to herein as “activation” of T cells. Therefore, CTL induction by an epitope of a polypeptide or peptide or combinations thereof can be evaluated by presenting an epitope of a polypeptide or peptide or combinations thereof to a T cell by APC, and detecting the induction of CTL. Furthermore, APCs have the effect of activating B cells, CD4+ T cells, CD8+ T cells, macrophages, eosinophils and NK cells.

A method for evaluating the inducing action of CTL using dendritic cells (DCs) as APC is well known in the art. DC is a representative APC having a robust CTL inducing action among APCs. In the methods of the invention, the epitope of a polypeptide or peptide or combinations thereof is initially expressed by the DC and then this DC is contacted with T cells. Detection of T cells having cytotoxic effects against the cells of interest after the contact with DC shows that the epitope of a polypeptide or peptide or combinations thereof has an activity of inducing the cytotoxic T cells. Furthermore, the induced immune response can also be examined by measuring IFN-gamma produced and released by CTL in the presence of antigen-presenting cells that carry immobilized peptide or a combination of peptides by visualizing using anti-IFN-gamma antibodies, such as an ELISPOT assay.

Apart from DC, peripheral blood mononuclear cells (PBMCs) may also be used as the APC. The induction of CTL is reported to be enhanced by culturing PBMC in the presence of GM-CSF and IL-4. Similarly, CTL has been shown to be induced by culturing PBMC in the presence of keyhole limpet hemocyanin (KLH) and IL-7.

The antigens confirmed to possess CTL-inducing activity by these methods are antigens having DC activation effect and subsequent CTL-inducing activity. Furthermore, CTLs that have acquired cytotoxicity due to presentation of the antigen by APC can be also used as vaccines against antigen-associated disorders.

The induction of immunity by expression of the influenza virus antigens can be further confirmed by observing the induction of antibody production against the influenza virus antigens. For example, when antibodies against an antigen are induced in a laboratory subject immunized with the composition encoding the antigens, and when antigen-associated pathology is suppressed by those antibodies, the composition is determined to induce immunity.

The specificity of the antibody response induced in a subject can include binding to many regions of the delivered antigen, as well as, the induction of neutralization capable antibodies that that prevent infection or reduce disease severity.

The induction of immunity by expression of the influenza virus antigens can be further confirmed by observing the induction of T cells, such as CD4+ T cells, CD8+ T cells, or a combination thereof. For example, CD4+ T cells can also lyse target cells, but mainly supply help in the induction of other types of immune responses, including CTL and antibody generation. The type of CD4+ T cell help can be characterized, as Th1, Th2, Th9, Th17, Tregulatory (Treg), or T follicular helper (Tfh) cells. Each subtype of CD4+ T cell supplies help to certain types of immune responses. In one embodiment, the composition selectively induces T follicular helper cells, which drive potent antibody responses.

The therapeutic compounds or compositions of the invention may be administered prophylactically (i.e., to prevent a disease or disorder) or therapeutically (i.e., to treat a disease or disorder) to subjects suffering from, or at risk of (or susceptible to) developing a disease or disorder. Such subjects may be identified using standard clinical methods. In the context of the present invention, prophylactic administration occurs prior to the manifestation of overt clinical symptoms of disease, such that a disease or disorder is prevented or alternatively delayed in its progression. In the context of the field of medicine, the term “prevent” encompasses any activity, which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

### Antigen

The present invention provides a composition that induces an immune response in a subject. In one embodiment, the composition comprises a combination of at least twenty influenza virus HA antigens from each of influenza A virus H1, H2, H3, H4, H5, H6, H7, H8, H9, H10, H11, H12, H13, H14, H15, H16, H17, and H18, and influenza B virus Vic and Yam. In some embodiments, the composition further comprises one or more additional nucleic acid molecules encoding at least 1, 2, 3, 4, 5, 6, 7, 8, 9, 10, 11, 12, 13, 14, 15, 16, 17, 18, 19, 20, or more than twenty additional viral antigens.

In one embodiment, the composition comprises a combination of LNPs comprising nucleic acid molecules, which a combination of at least twenty influenza virus HA antigens from each of influenza A virus H1, H2, H3, H4, H5, H6, H7, H8, H9, H10, H11, H12, H13, H14, H15, H16, H17, and H18, and influenza B virus Vic and Yam. In some embodiments, the vaccine further comprises one or more additional nucleic acid molecules encoding at least 1, 2, 3, 4, 5, 6, 7, 8, 9, 10, 11, 12, 13, 14, 15, 16, 17, 18, 19, 20, or more than twenty additional viral antigens. In some embodiments, one or more additional viral antigens may be from human immunodeficiency virus (HIV), Chikungunya virus (CHIKV), dengue fever virus, papilloma viruses, for example, human papillomoa virus (HPV), polio virus, hepatitis viruses, for example, hepatitis A virus (HAV), hepatitis B virus (HBV), hepatitis C virus (HCV), hepatitis D virus (HDV), and hepatitis E virus (HEV), smallpox virus (Variola major and minor), vaccinia virus, rhinoviruses, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, lassa virus, arenavirus, Merkel cell polyoma virus (MCV), influenza virus, a coronavirus including but not limited to severe acute respiratory syndrome-coronavirus 2, a cancer causing virus, or any combination thereof. In one embodiment, one or more additional viral antigen are influenza viral antigens. Exemplary additional influenza virus antigens include, but are not limited to, a full length HA antigen or a fragment thereof, an HA-stalk domain or a fragment thereof, an HA-head domain or a fragment thereof, a full length neuraminidase (NA) antigen or a fragment thereof, a NA-stalk domain or a fragment thereof, NA-head domain or a fragment thereof, full length NP antigen or a fragment thereof, full length matrix protein 1 (M1) antigen or a fragment thereof, full length matrix-2 (M2) ion channel antigen or a fragment thereof, a M2 ion channel-extracellular domain or a fragment thereof, a M2 ion channel-intracellular domain or a fragment thereof, or any combination thereof.

For example, in some embodiments, the composition comprises a nucleoside-modified RNA encoding a combination of at least twenty influenza virus HA antigens from each of influenza A virus H1, H2, H3, H4, H5, H6, H7, H8, H9, H10, H11, H12, H13, H14, H15, H16, H17, and H18, and influenza B virus Vic and Yam, or fragments or variants thereof.

In some embodiments, the influenza virus HA antigen comprises a full length HA antigen, or a fragment or variant thereof, an HA-stalk domain, or a fragment or variant thereof, an HA-head domain, or a fragment or variant thereof, an HA-headless domain, or a fragment or variant thereof, an optimized full length HA antigen, or a fragment or variant thereof, an optimized HA domain, or a fragment or variant thereof, a mini HA domain, or a fragment or variant thereof, or any combination thereof.

In one embodiment, the mRNA molecules encoding the HA antigens correspond to the nucleotide sequences set forth in: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20. In one embodiment, the mRNA molecules encoding the HA antigens are encoded by DNA sequences as set forth in: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20. Other amino acid sequences for HA antigens are known in the art, including but not limited to, amino acid sequences for HA-headless domains (see, e.g., U.S. Pat. No. 9,051,359 and U.S. Patent Application Publication No. 2019/0314490 A1) and amino acid sequences for mini HA domains (see e.g., International Publication No. WO 2014/191435 A1), each of which is incorporated herein in its entirety by reference.

In one embodiment, the nucleic acid molecule encoding the HA antigen comprises a sequence encoding a tag or signal peptide (SP). Other signal peptides that may be used include, but are not limited to, signal sequences derived from IL-2, tPA, mouse and human IgG, and synthetic optimized signal sequences. In some instances, the nucleic acid sequence comprises include additional sequences that encode linker or tag sequences that are linked to the antigen by a peptide bond.

In some embodiments, the influenza virus antigen comprises an amino acid sequence that is substantially homologous to the amino acid sequence of an influenza virus antigen described herein and retains the immunogenic function of the original amino acid sequence. For example, in some embodiments, the nucleotide sequence of the nucleic acid molecule encoding the influenza virus antigen has a degree of identity with respect to the original nucleotide sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

### Adjuvant

In one embodiment, the composition comprises an adjuvant. In one embodiment, the composition comprises a nucleic acid molecule encoding an adjuvant. In one embodiment, the adjuvant-encoding nucleic acid molecule is IVT RNA. In one embodiment, the adjuvant-encoding nucleic acid molecule is nucleoside-modified RNA. In one embodiment, the adjuvant-encoding nucleic acid molecule is nucleoside-modified mRNA.

Exemplary adjuvants include, but are not limited to, alpha-interferon, gamma-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86. Other genes which may be useful adjuvants include those encoding: MCP-I, MIP-Ia, MIP-Ip, IL-8, RANTES, L-selectin, P-selectin, E-selectin, CD34, GlyCAM-1, MadCAM-1, LFA-I, VLA-I, Mac-1, pl50.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R2, TRICK2, DR6, Caspase ICE, Fos, c-jun, Sp-I, Ap-I, Ap-2, p38, p65Rel, MyD88, IRAK, TRAF6, IkB, Inactive NIK, SAP K, SAP-I, INK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP 1, TAP2, anti-CTLA4-sc, anti-LAG3-Ig, anti-TIM3-Ig, and functional fragments thereof.

In some embodiments, the composition comprises an LNP, where the LNP acts as an adjuvant.

### Nucleic Acids

In one embodiment, the invention includes a combination of nucleic acid molecules encoding HA antigens for each of influenza A virus H1, H2, H3, H4, H5, H6, H7, H8, H9, H10, H11, H12, H13, H14, H15, H16, H17, and H18, and influenza B virus Vic and Yam. In one embodiment, the invention includes a nucleoside-modified nucleic acid molecules. In one embodiment, the nucleoside-modified nucleic acid molecules encode HA antigens for each of influenza A virus H1, H2, H3, H4, H5, H6, H7, H8, H9, H10, H11, H12, H13, H14, H15, H16, H17, and H18, and influenza B virus Vic and Yam.

The nucleic acid molecule can be made using any methodology in the art, including, but not limited to, in vitro transcription, chemical synthesis, or the like.

The nucleotide sequences encoding the combination of influenza virus antigens as described herein, can alternatively comprise sequence variations with respect to the original nucleotide sequences, for example, substitutions, insertions and/or deletions of one or more nucleotides, with the condition that the resulting polynucleotide encodes a polypeptide according to the invention. Therefore, the scope of the present invention includes nucleotide sequences that are substantially homologous or substantially identical to the nucleotide sequences recited herein and encode the influenza virus HA antigens of the invention.

A nucleotide sequence that is substantially homologous to a nucleotide sequence encoding an antigen can typically be isolated from a producer organism of the antigen based on the information contained in the nucleotide sequence by means of introducing conservative or non-conservative substitutions, for example. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art.

Further, the scope of the invention includes nucleotide sequences that encode amino acid sequences that are substantially homologous to the amino acid sequences recited herein and preserve the immunogenic function of the original amino acid sequence.

In one embodiment, the invention relates to a construct, comprising a nucleotide sequence encoding an influenza virus antigen. In one embodiment, the construct comprises a plurality of nucleotide sequences encoding a plurality of influenza virus antigens. For example, in some embodiments, the construct encodes 1 or more, 2 or more, 3 or more, or all influenza virus antigens. In one embodiment, the invention relates to a construct, comprising a nucleotide sequence encoding an adjuvant. In one embodiment, the construct comprises a first nucleotide sequence encoding an influenza virus antigen and a second nucleotide sequence encoding an adjuvant.

In one embodiment, the composition comprises a plurality of constructs, each construct encoding an influenza virus HA antigen for each of influenza A virus H1, H2, H3, H4, H5, H6, H7, H8, H9, H10, H11, H12, H13, H14, H15, H16, H17, and H18, and influenza B virus Vic and Yam. In one embodiment, the composition comprises a first construct, comprising a nucleotide sequence encoding an influenza virus antigen; and a second construct, comprising a nucleotide sequence encoding an adjuvant.

In another particular embodiment, the construct is operatively bound to a translational control element. The construct can incorporate an operatively bound regulatory sequence for the expression of the nucleotide sequence of the invention, thus forming an expression cassette.

### Vectors

The nucleic acid sequences coding for the influenza virus antigens of the invention can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically.

The nucleic acid molecules can be cloned into a number of types of vectors. For example, the nucleic acid can be cloned into a vector including, but not limited to a plasmid, a phagemid, a phage derivative, an animal virus, a PCR-generated linear DNA sequence, and a cosmid. Vectors of particular interest include expression vectors, replication vectors, probe generation vectors, sequencing vectors and vectors optimized for in vitro transcription.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, carbohydrates, peptides, cationic polymers, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/RNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as it is more readily evaporated than methanol.

Regardless of the method used to introduce exogenous nucleic acids into a host cell or otherwise expose a cell to a composition of the present invention, in order to confirm the presence of the mRNA sequence in the host cell, a variety of assays may be performed. Such assays include, for example, “molecular biological” assays well known to those of skill in the art, such as Northern blotting and RT-PCR; “biochemical” assays, such as detecting the presence or absence of a particular peptide, e.g., by immunogenic means (ELISAs and Western blots) or by assays described herein to identify agents falling within the scope of the invention.

### In Vitro Transcribed RNA

In one embodiment, the composition of the invention comprises a combination of in vitro transcribed (IVT) RNA molecules encoding the influenza virus antigens of the invention. In one embodiment, an IVT RNA can be introduced to a cell as a form of transient transfection. The RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. In one embodiment, the desired template for in vitro transcription is an influenza virus antigen capable of inducing an adaptive immune response. In one embodiment, the desired template for in vitro transcription is an adjuvant capable of enhancing an adaptive immune response.

In one embodiment, the DNA to be used for PCR contains an open reading frame. The DNA can be from a naturally occurring DNA sequence from the genome of an organism. In one embodiment, the DNA is a full-length gene of interest of a portion of a gene. The gene can include some or all of the 5′ and/or 3′ untranslated regions (UTRs). The gene can include exons and introns. In one embodiment, the DNA to be used for PCR is a human gene. In another embodiment, the DNA to be used for PCR is a human gene including the 5′ and 3′ UTRs. In another embodiment, the DNA to be used for PCR is a gene from a pathogenic or commensal organism, including bacteria, viruses, parasites, and fungi. In another embodiment, the DNA to be used for PCR is from a pathogenic or commensal organism, including bacteria, viruses, parasites, and fungi, including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

Genes that can be used as sources of DNA for PCR include genes that encode polypeptides that induce or enhance an adaptive immune response in an organism. In some instances, the genes are useful for a short term treatment. In some instances, the genes have limited safety concerns regarding dosage of the expressed gene.

In various embodiments, a plasmid is used to generate a template for in vitro transcription of mRNA, which is used for transfection.

Chemical structures with the ability to promote stability and/or translation efficiency may also be used. In some embodiments, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 RNA polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability of mRNA in the cell. On a circular DNA template, for instance, plasmid DNA, RNA polymerase produces a long concatameric product, which is not suitable for expression in eukaryotic cells. The transcription of plasmid DNA linearized at the end of the 3′ UTR results in normal sized mRNA, which is effective in eukaryotic transfection when it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003)).

The conventional method of integration of polyA/T stretches into a DNA template is molecular cloning. However, polyA/T sequence integrated into plasmid DNA can cause plasmid instability, which can be ameliorated through the use of recombination incompetent bacterial cells for plasmid propagation.

Poly(A) tails of RNAs can be further extended following in vitro transcription with the use of a poly(A) polymerase, such as E. coli polyA polymerase (E-PAP) or yeast polyA polymerase. In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to mRNA molecules. In one embodiment, RNAs produced by the methods to include a 5′ cap1 structure. Such cap1 structure can be generated using Vaccinia capping enzyme and 2′-O-methyltransferase enzymes (CellScript, Madison, WI). Alternatively, 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001)). In some embodiments RNA of the invention is introduced to a cell with a method comprising the use of TransIT®-mRNA transfection Kit (Mirus, Madison WI), which, in some instances, provides high efficiency, low toxicity, transfection.

### Nucleoside-Modified RNA

In one embodiment, the composition of the present invention comprises a nucleoside-modified nucleic acid encoding an influenza virus antigen as described herein. In one embodiment, the composition of the present invention comprises a nucleoside-modified nucleic acid encoding a plurality of antigens, including one or more influenza virus antigens. In one embodiment, the composition of the present invention comprises a nucleoside-modified nucleic acid encoding an adjuvant as described herein. In one embodiment, the composition of the present invention comprises a nucleoside-modified nucleic acid encoding one or more influenza virus antigens and one or more adjuvants.

In one embodiment, the composition of the present invention comprises a series of nucleoside-modified nucleic acid encoding one or more influenza virus antigens that change for each subsequent injection to follow a lineage scheme.

For example, in one embodiment, the composition comprises a nucleoside-modified RNA. In one embodiment, the composition comprises a nucleoside-modified mRNA. Nucleoside-modified mRNA have particular advantages over non-modified mRNA, including for example, increased stability, low or absent innate immunogenicity, and enhanced translation. Nucleoside-modified mRNA useful in the present invention is further described in U.S. Pat. Nos. 8,278,036, 8,691,966, and 8,835,108, each of which is incorporated by reference herein in its entirety.

In some embodiments, nucleoside-modified mRNA does not activate any pathophysiologic pathways, translates very efficiently and almost immediately following delivery, and serve as templates for continuous protein production in vivo lasting for several days to weeks (Karikó et al., 2008, Mol Ther 16:1833-1840; Karikó et al., 2012, Mol Ther 20:948-953). The amount of mRNA required to exert a physiological effect is small, making it applicable for human therapy. For example, as described herein, nucleoside-modified mRNA encoding an influenza virus antigen has demonstrated the ability to induce antigen-specific antibody production. For example, in some instances, antigen encoded by nucleoside-modified mRNA induces greater production of antigen-specific antibody production as compared to antigen encoded by non-modified mRNA.

In some instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In some embodiments, using mRNA rather than the protein also has many advantages. Half-lives of proteins in the circulation or in tissues are often short, thus protein treatment would need frequent dosing, while mRNA provides a template for continuous protein production for several days to weeks. Purification of proteins is problematic and they can contain aggregates and other impurities that cause adverse effects (Kromminga and Schellekens, 2005, Ann NY Acad Sci 1050:257-265).

In some embodiments, the nucleoside-modified RNA comprises the naturally occurring modified-nucleoside pseudouridine. In some embodiments, inclusion of pseudouridine makes the mRNA more stable, non-immunogenic, and highly translatable (Karikó et al., 2008, Mol Ther 16:1833-1840; Anderson et al., 2010, Nucleic Acids Res 38:5884-5892; Anderson et al., 2011, Nucleic Acids Research 39:9329-9338; Karikó et al., 2011, Nucleic Acids Research 39:e142; Karikó et al., 2012, Mol Ther 20:948-953; Karikó et al., 2005, Immunity 23:165-175).

It has been demonstrated that the presence of modified nucleosides, including pseudouridines in RNA suppress their innate immunogenicity (Karikó et al., 2005, Immunity 23:165-175). Further, protein-encoding, in vitro-transcribed RNA containing pseudouridine can be translated more efficiently than RNA containing no or other modified nucleosides (Karikó et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010, Nucleic Acids Res 38:5884-5892).

Similar effects as described for pseudouridine have also been observed for RNA containing 1-methyl-pseudouridine.

In some embodiments, the nucleoside-modified nucleic acid molecule is a purified nucleoside-modified nucleic acid molecule. For example, in some embodiments, the composition is purified to remove double-stranded contaminants. In some instances, a preparative high-performance liquid chromatography (HPLC) purification procedure is used to obtain pseudouridine-containing RNA that has superior translational potential and no innate immunogenicity (Karikó et al., 2011, Nucleic Acids Research 39:e142). Administering HPLC-purified, pseudouridine-containing RNA coding for erythropoietin into mice and macaques resulted in a significant increase of serum EPO levels (Karikó et al., 2012, Mol Ther 20:948-953), thus confirming that pseudouridine-containing mRNA is suitable for in vivo protein therapy. In some embodiments, the nucleoside-modified nucleic acid molecule is purified using non-HPLC methods. In some instances, the nucleoside-modified nucleic acid molecule is purified using chromatography methods, including but not limited to HPLC and fast protein liquid chromatography (FPLC). An exemplary FPLC-based purification procedure is described in Weissman et al., 2013, Methods Mol Biol, 969: 43-54. Exemplary purification procedures are also described in U.S. Patent Application Publication No. US2016/0032316, which is hereby incorporated by reference in its entirety.

The present invention encompasses RNA, oligoribonucleotide, and polyribonucleotide molecules comprising pseudouridine or a modified nucleoside. In some embodiments, the composition comprises an isolated nucleic acid encoding an antigen, wherein the nucleic acid comprises a pseudouridine or a modified nucleoside. In some embodiments, the composition comprises a vector, comprising an isolated nucleic acid encoding an antigen, adjuvant, or combination thereof, wherein the nucleic acid comprises a pseudouridine or a modified nucleoside.

In one embodiment, the nucleoside-modified RNA of the invention is IVT RNA, as described elsewhere herein. For example, in some embodiments, the nucleoside-modified RNA is synthesized by T7 phage RNA polymerase. In another embodiment, the nucleoside-modified mRNA is synthesized by SP6 phage RNA polymerase. In another embodiment, the nucleoside-modified RNA is synthesized by T3 phage RNA polymerase.

In one embodiment, the modified nucleoside is m¹acp³Ψ (1-methyl-3-(3-amino-3-carboxypropyl) pseudouridine. In another embodiment, the modified nucleoside is m¹Ψ (1-methylpseudouridine). In another embodiment, the modified nucleoside is Ψm (2′-O-methylpseudouridine). In another embodiment, the modified nucleoside is m⁵D (5-methyldihydrouridine). In another embodiment, the modified nucleoside is m³Ψ (3-methylpseudouridine). In another embodiment, the modified nucleoside is a pseudouridine moiety that is not further modified. In another embodiment, the modified nucleoside is a monophosphate, diphosphate, or triphosphate of any of the above pseudouridines. In another embodiment, the modified nucleoside is any other pseudouridine-like nucleoside known in the art.

In another embodiment, the nucleoside that is modified in the nucleoside-modified RNA the present invention is uridine (U). In another embodiment, the modified nucleoside is cytidine (C). In another embodiment, the modified nucleoside is adenosine (A). In another embodiment, the modified nucleoside is guanosine (G).

In another embodiment, the modified nucleoside of the present invention is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine).

In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²i6A (2-methylthio-N⁶isopentenyladenosine); io⁶A (N⁶-(cis-hydroxyisopentenyl)adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²₂G (N²,N²-dimethylguanosine); m²Gm (N²,2′-O-dimethylguanosine); m²₂Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-Carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶₂Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In another embodiment, a nucleoside-modified RNA of the present invention comprises a combination of 2 or more of the above modifications. In another embodiment, the nucleoside-modified RNA comprises a combination of 3 or more of the above modifications. In another embodiment, the nucleoside-modified RNA comprises a combination of more than 3 of the above modifications.

In various embodiments, between 0.1% and 100% of the residues in the nucleoside-modified RNA of the present invention are modified (e.g., either by the presence of pseudouridine, 1-methyl-pseudouridine, 5-methyl-uridine or another modified nucleoside base). In one embodiment, the fraction of modified residues is 0.1%. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.7%. In another embodiment, the fraction is 0.8%. In another embodiment, the fraction is 0.9%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 7%. In another embodiment, the fraction is 8%. In another embodiment, the fraction is 9%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 55%. In another embodiment, the fraction is 60%. In another embodiment, the fraction is 65%. In another embodiment, the fraction is 70%. In another embodiment, the fraction is 75%. In another embodiment, the fraction is 80%. In another embodiment, the fraction is 85%. In another embodiment, the fraction is 90%. In another embodiment, the fraction is 91%. In another embodiment, the fraction is 92%. In another embodiment, the fraction is 93%. In another embodiment, the fraction is 94%. In another embodiment, the fraction is 95%. In another embodiment, the fraction is 96%. In another embodiment, the fraction is 97%. In another embodiment, the fraction is 98%. In another embodiment, the fraction is 99%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, 0.1% of the residues of a given nucleoside (i.e., uridine, cytidine, guanosine, or adenosine) are modified. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.7%. In another embodiment, the fraction is 0.8%. In another embodiment, the fraction is 0.9%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 7%. In another embodiment, the fraction is 8%. In another embodiment, the fraction is 9%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 55%. In another embodiment, the fraction is 60%. In another embodiment, the fraction is 65%. In another embodiment, the fraction is 70%. In another embodiment, the fraction is 75%. In another embodiment, the fraction is 80%. In another embodiment, the fraction is 85%. In another embodiment, the fraction is 90%. In another embodiment, the fraction is 91%. In another embodiment, the fraction is 92%. In another embodiment, the fraction is 93%. In another embodiment, the fraction is 94%. In another embodiment, the fraction is 95%. In another embodiment, the fraction is 96%. In another embodiment, the fraction is 97%. In another embodiment, the fraction is 98%. In another embodiment, the fraction is 99%. In another embodiment, the fraction is 100%. In another embodiment, the fraction of the given nucleotide that is modified is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In some embodiments, the composition comprises a purified preparation of single-stranded nucleoside modified RNA. For example, in some embodiments, the purified preparation of single-stranded nucleoside modified RNA is substantially free of double stranded RNA (dsRNA). In some embodiments, the purified preparation is at least 90%, or at least 91%, or at least 92%, or at least 93% or at least 94%, or at least 95%, or at least 96%, or at least 97%, or at least 98%, or at least 99%, or at least 99.5%, or at least 99.9% single stranded nucleoside modified RNA, relative to all other nucleic acid molecules (DNA, dsRNA, etc.).

In another embodiment, a nucleoside-modified RNA of the present invention is translated in the cell more efficiently than an unmodified RNA molecule with the same sequence. In another embodiment, the nucleoside-modified RNA exhibits enhanced ability to be translated by a target cell. In another embodiment, translation is enhanced by a factor of 2-fold relative to its unmodified counterpart. In another embodiment, translation is enhanced by a 3-fold factor. In another embodiment, translation is enhanced by a 4-fold factor. In another embodiment, translation is enhanced by a 5-fold factor. In another embodiment, translation is enhanced by a 6-fold factor. In another embodiment, translation is enhanced by a 7-fold factor. In another embodiment, translation is enhanced by an 8-fold factor. In another embodiment, translation is enhanced by a 9-fold factor. In another embodiment, translation is enhanced by a 10-fold factor. In another embodiment, translation is enhanced by a 15-fold factor. In another embodiment, translation is enhanced by a 20-fold factor. In another embodiment, translation is enhanced by a 50-fold factor. In another embodiment, translation is enhanced by a 100-fold factor. In another embodiment, translation is enhanced by a 200-fold factor. In another embodiment, translation is enhanced by a 500-fold factor. In another embodiment, translation is enhanced by a 1000-fold factor. In another embodiment, translation is enhanced by a 2000-fold factor. In another embodiment, the factor is 10-1000-fold. In another embodiment, the factor is 10-100-fold. In another embodiment, the factor is 10-200-fold. In another embodiment, the factor is 10-300-fold. In another embodiment, the factor is 10-500-fold. In another embodiment, the factor is 20-1000-fold. In another embodiment, the factor is 30-1000-fold. In another embodiment, the factor is 50-1000-fold. In another embodiment, the factor is 100-1000-fold. In another embodiment, the factor is 200-1000-fold. In another embodiment, translation is enhanced by any other significant amount or range of amounts.

In another embodiment, the nucleoside-modified antigen-encoding RNA of the present invention induces a significantly more robust adaptive immune response as compared with an unmodified in vitro-synthesized RNA molecule of the same sequence. In another embodiment, the modified RNA molecule induces an adaptive immune response that is 2-fold greater than its unmodified counterpart. In another embodiment, the adaptive immune response is increased by a 3-fold factor. In another embodiment, the adaptive immune response is increased by a 4-fold factor. In another embodiment, the adaptive immune response is increased by a 5-fold factor. In another embodiment, the adaptive immune response is increased by a 6-fold factor. In another embodiment, the adaptive immune response is increased by a 7-fold factor. In another embodiment, the adaptive immune response is increased by an 8-fold factor. In another embodiment, the adaptive immune response is increased by a 9-fold factor. In another embodiment, the adaptive immune response is increased by a 10-fold factor. In another embodiment, the adaptive immune response is increased by a 15-fold factor. In another embodiment, the adaptive immune response is increased by a 20-fold factor. In another embodiment, the adaptive immune response is increased by a 50-fold factor. In another embodiment, the adaptive immune response is increased by a 100-fold factor. In another embodiment, the adaptive immune response is increased by a 200-fold factor. In another embodiment, the adaptive immune response is increased by a 500-fold factor. In another embodiment, the adaptive immune response is increased by a 1000-fold factor. In another embodiment, the adaptive immune response is increased by a 2000-fold factor. In another embodiment, the adaptive immune response is increased by another fold difference.

In another embodiment, “induces significantly more robust adaptive immune response” refers to a detectable increase in an adaptive immune response. In another embodiment, the term refers to a fold increase in the adaptive immune response (e.g., 1 of the fold increases enumerated above). In another embodiment, the term refers to an increase such that the nucleoside-modified RNA can be administered at a lower dose or frequency than an unmodified RNA molecule while still inducing a similarly effective adaptive immune response. In another embodiment, the increase is such that the nucleoside-modified RNA can be administered using a single dose to induce an effective adaptive immune response.

In another embodiment, the nucleoside-modified RNA of the present invention exhibits significantly less innate immunogenicity than an unmodified in vitro-synthesized RNA molecule of the same sequence. In another embodiment, the modified RNA molecule exhibits an innate immune response that is 2-fold less than its unmodified counterpart. In another embodiment, innate immunogenicity is reduced by a 3-fold factor. In another embodiment, innate immunogenicity is reduced by a 4-fold factor. In another embodiment, innate immunogenicity is reduced by a 5-fold factor. In another embodiment, innate immunogenicity is reduced by a 6-fold factor. In another embodiment, innate immunogenicity is reduced by a 7-fold factor. In another embodiment, innate immunogenicity is reduced by a 8-fold factor. In another embodiment, innate immunogenicity is reduced by a 9-fold factor. In another embodiment, innate immunogenicity is reduced by a 10-fold factor. In another embodiment, innate immunogenicity is reduced by a 15-fold factor. In another embodiment, innate immunogenicity is reduced by a 20-fold factor. In another embodiment, innate immunogenicity is reduced by a 50-fold factor. In another embodiment, innate immunogenicity is reduced by a 100-fold factor. In another embodiment, innate immunogenicity is reduced by a 200-fold factor. In another embodiment, innate immunogenicity is reduced by a 500-fold factor. In another embodiment, innate immunogenicity is reduced by a 1000-fold factor. In another embodiment, innate immunogenicity is reduced by a 2000-fold factor. In another embodiment, innate immunogenicity is reduced by another fold difference.

In another embodiment, “exhibits significantly less innate immunogenicity” refers to a detectable decrease in innate immunogenicity. In another embodiment, the term refers to a fold decrease in innate immunogenicity (e.g., 1 of the fold decreases enumerated above). In another embodiment, the term refers to a decrease such that an effective amount of the nucleoside-modified RNA can be administered without triggering a detectable innate immune response. In another embodiment, the term refers to a decrease such that the nucleoside-modified RNA can be repeatedly administered without eliciting an innate immune response sufficient to detectably reduce production of the protein encoded by the modified RNA. In another embodiment, the decrease is such that the nucleoside-modified RNA can be repeatedly administered without eliciting an innate immune response sufficient to eliminate detectable production of the protein encoded by the modified RNA.

### Lipid Nanoparticle

In one embodiment, delivery of nucleoside-modified RNA comprises any suitable delivery method, including exemplary RNA transfection methods described elsewhere herein. In some embodiments, delivery of a nucleoside-modified RNA to a subject comprises mixing the nucleoside-modified RNA with a transfection reagent prior to the step of contacting. In another embodiment, a method of present invention further comprises administering nucleoside-modified RNA together with the transfection reagent. In another embodiment, the transfection reagent is a cationic lipid reagent. In another embodiment, the transfection reagent is a cationic polymer reagent.

In another embodiment, the transfection reagent is a lipid-based transfection reagent. In another embodiment, the transfection reagent is a protein-based transfection reagent. In another embodiment, the transfection reagent is a carbohydrate-based transfection reagent. In another embodiment, the transfection reagent is a cationic lipid-based transfection reagent. In another embodiment, the transfection reagent is a cationic polymer-based transfection reagent. In another embodiment, the transfection reagent is a polyethyleneimine based transfection reagent. In another embodiment, the transfection reagent is calcium phosphate. In another embodiment, the transfection reagent is Lipofectin®, Lipofectamine®, or TransIT®. In another embodiment, the transfection reagent is any other transfection reagent known in the art.

In another embodiment, the transfection reagent forms a liposome. Liposomes, in another embodiment, increase intracellular stability, increase uptake efficiency and improve biological activity. In another embodiment, liposomes are hollow spherical vesicles composed of lipids arranged in a similar fashion as those lipids, which make up the cell membrane. They have, in another embodiment, an internal aqueous space for entrapping water-soluble compounds and range in size from 0.05 to several microns in diameter. In another embodiment, liposomes can deliver RNA to cells in a biologically active form.

In one embodiment, the composition comprises a lipid nanoparticle (LNP) and one or more nucleic acid molecules described herein. For example, in one embodiment, the composition comprises an LNP and one or more nucleoside-modified RNA molecules encoding one or more antigens, adjuvants, or a combination thereof.

In some embodiments, the lipid nanoparticle is a particle having at least one dimension on the order of nanometers (e.g., 1-1,000 nm). In some embodiments, the lipid nanoparticle comprises one or more lipids. For example, in some embodiments, the lipid comprises a lipid of Formula (I), (II) or (III).

In some embodiments, lipid nanoparticles are included in a formulation comprising a nucleoside-modified RNA as described herein. In some embodiments, such lipid nanoparticles comprise a cationic lipid (e.g., a lipid of Formula (I), (II) or (III)) and one or more excipient selected from neutral lipids, charged lipids, steroids and polymer conjugated lipids (e.g., a pegylated lipid such as a pegylated lipid of structure (IV). In some embodiments, the nucleoside-modified RNA is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In some embodiments, the nucleoside-modified RNA, when present in the lipid nanoparticles, is resistant in aqueous solution to degradation with a nuclease.

The LNP may comprise any lipid capable of forming a particle to which the one or more nucleic acid molecules are attached, or in which the one or more nucleic acid molecules are encapsulated.

In one embodiment, the LNP comprises one or more cationic lipids, and one or more stabilizing lipids. Stabilizing lipids include neutral lipids and pegylated lipids.

In one embodiment, the LNP comprises a cationic lipid. In some embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

Suitable amino lipids include those having the formula:

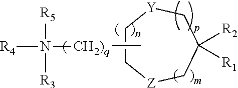

wherein R₁ and R₂ are either the same or different and independently optionally substituted C₁₀-C₂₄ alkyl, optionally substituted C₁₀-C₂₄ alkenyl, optionally substituted C₁₀-C₂₄ alkynyl, or optionally substituted C₁₀-C₂₄ acyl;
R₃ and R₄ are either the same or different and independently optionally substituted C₁-C₆ alkyl, optionally substituted C₂-C₆ alkenyl, or optionally substituted C₂-C₆ alkynyl or R₃ and R₄ may join to form an optionally substituted heterocyclic ring of 4 to 6 carbon atoms and 1 or 2 heteroatoms chosen from nitrogen and oxygen;
R₅ is either absent or present and when present is hydrogen or C₁-C₆ alkyl;
m, n, and p are either the same or different and independently either 0 or 1 with the proviso that m, n, and p are not simultaneously 0;
q is 0, 1, 2, 3, or 4; and
Y and Z are either the same or different and independently O, S, or NH.

In one embodiment, R₁ and R₂ are each linoleyl, and the amino lipid is a dilinoleyl amino lipid. In one embodiment, the amino lipid is a dilinoleyl amino lipid.

A representative useful dilinoleyl amino lipid has the formula:

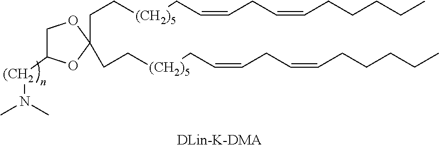

wherein n is 0, 1, 2, 3, or 4.

In one embodiment, the cationic lipid is a DLin-K-DMA. In one embodiment, the cationic lipid is DLin-KC2-DMA (DLin-K-DMA above, wherein n is 2).

In one embodiment, the cationic lipid component of the LNPs has the structure of Formula (I):

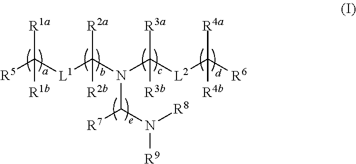

or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl;
R⁸ and R⁹ are each independently C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24;
b and c are each independently an integer from 1 to 24; and
e is 1 or 2.

In some embodiments of Formula (I), at least one of R^{1a}, R^{2a}, R^{3a} or R^{4a} is C₁-C₁₂ alkyl, or at least one of L¹ or L² is —O(C═O)— or —(C═O)O—. In other embodiments, R^{1a} and R^{1b} are not isopropyl when a is 6 or n-butyl when a is 8.

In still further embodiments of Formula (I), at least one of R^{1a}, R^{2a}, R^{3a} or R^{4a} is C₁-C₁₂ alkyl, or at least one of L¹ or L² is —O(C═O)— or —(C═O)O—; and
R^{1a} and R^{1b} are not isopropyl when a is 6 or n-butyl when a is 8.

In other embodiments of Formula (I), R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;

In some embodiments of Formula (I), any one of L¹ or L² may be —O(C═O)— or a carbon-carbon double bond. L¹ and L² may each be —O(C═O)— or may each be a carbon-carbon double bond.

In some embodiments of Formula (I), one of L¹ or L² is —O(C═O)—. In other embodiments, both L¹ and L² are —O(C═O)—.

In some embodiments of Formula (I), one of L¹ or L² is —(C═O)O—. In other embodiments, both L¹ and L² are —(C═O)O—.

In some other embodiments of Formula (I), one of L¹ or L² is a carbon-carbon double bond. In other embodiments, both L¹ and L² are a carbon-carbon double bond.

In still other embodiments of Formula (I), one of L¹ or L² is —O(C═O)— and the other of L¹ or L² is —(C═O)O—. In more embodiments, one of L¹ or L² is —O(C═O)— and the other of L¹ or L² is a carbon-carbon double bond. In yet more embodiments, one of L¹ or L² is —(C═O)O— and the other of L¹ or L² is a carbon-carbon double bond.

It is understood that “carbon-carbon” double bond, as used throughout the specification, refers to one of the following structures:

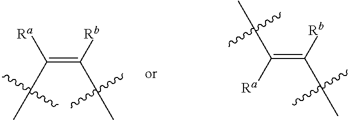

wherein R^{a} and R^{b} are, at each occurrence, independently H or a substituent. For example, in some embodiments R^{a} and R^{b} are, at each occurrence, independently H, C₁-C₁₂ alkyl or cycloalkyl, for example H or C₁-C₁₂ alkyl.

In other embodiments, the lipid compounds of Formula (I) have the following structure (Ia):

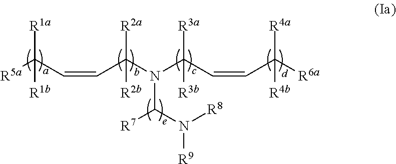

In other embodiments, the lipid compounds of Formula (I) have the following structure (Ib):

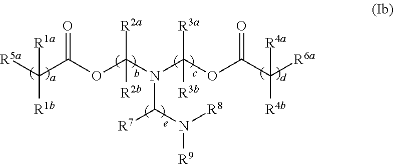

In yet other embodiments, the lipid compounds of Formula (I) have the following structure (Ic):

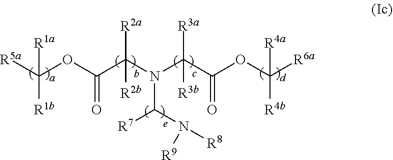

In some embodiments of the lipid compound of Formula (I), a, b, c and d are each independently an integer from 2 to 12 or an integer from 4 to 12. In other embodiments, a, b, c and d are each independently an integer from 8 to 12 or 5 to 9. In some embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some other embodiments of Formula (I), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some more embodiments of Formula (I), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some other embodiments of Formula (I), d is 0. In some embodiments, d is 1. In other embodiments, d is 2. In more embodiments, d is 3. In yet other embodiments, d is 4. In some embodiments, d is 5. In other embodiments, d is 6. In more embodiments, d is 7. In yet other embodiments, d is 8. In some embodiments, d is 9. In other embodiments, d is 10. In more embodiments, d is 11. In yet other embodiments, d is 12. In some embodiments, d is 13. In other embodiments, d is 14. In more embodiments, d is 15. In yet other embodiments, d is 16.

In some other various embodiments of Formula (I), a and d are the same. In some other embodiments, b and c are the same. In some other specific embodiments, a and d are the same and b and c are the same.

The sum of a and b and the sum of c and d in Formula (I) are factors which may be varied to obtain a lipid of Formula (I) having the desired properties. In one embodiment, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a. b, c and d are selected such the sum of a and b and the sum of c and d is 12 or greater.

In some embodiments of Formula (I), e is 1. In other embodiments, e is 2.

The substituents at R^{1a}, R^{2a}, R^{3a} and R^{4a} of Formula (I) are not particularly limited. In some embodiments R^{1a}, R^{2a}, R^{3a} and R^{4a} are H at each occurrence. In some other embodiments at least one of R^{1a}, R^{2a}, R^{3a} and R^{4a} is C₁-C₁₂ alkyl. In some other embodiments at least one of R^{1a}, R^{2a}, R^{3a} and R^{4a} is C₁-C₈ alkyl. In some other embodiments at least one of R^{1a}, R^{2a}, R^{3a} and R^{4a} is C₁-C₆ alkyl. In some of the foregoing embodiments, the C₁-C₈ alkyl is methyl, ethyl, n-propyl, iso-propyl, n-butyl, iso-butyl, tert-butyl, n-hexyl or n-octyl.

In some embodiments of Formula (I), R^{1a}, R^{1b}, R^{4a} and R^{4b} are C₁-C₁₂ alkyl at each occurrence.

In further embodiments of Formula (I), at least one of R^{1b}, R^{2b}, R^{3b} and R^{4b} is H or R^{1b}, R^{2b}, R^{3b} and R^{4b} are H at each occurrence.

In some embodiments of Formula (I), R^{1b} together with the carbon atom to which it is bound is taken together with an adjacent R^{1b} and the carbon atom to which it is bound to form a carbon-carbon double bond. In other embodiments of the foregoing R^{4b} together with the carbon atom to which it is bound is taken together with an adjacent R^{4b} and the carbon atom to which it is bound to form a carbon-carbon double bond.

The substituents at R⁵ and R⁶ of Formula (I) are not particularly limited in the foregoing embodiments. In some embodiments one or both of R⁵ or R⁶ is methyl. In some other embodiments one or both of R⁵ or R⁶ is cycloalkyl for example cyclohexyl. In these embodiments the cycloalkyl may be substituted or not substituted. In some other embodiments the cycloalkyl is substituted with C₁-C₁₂ alkyl, for example tert-butyl.

The substituents at R⁷ are not particularly limited in the foregoing embodiments of Formula (I). In some embodiments at least one R⁷ is H. In some other embodiments, R⁷ is H at each occurrence. In some other embodiments R⁷ is C₁-C₁₂ alkyl.

In some other of the foregoing embodiments of Formula (I), one of R⁸ or R⁹ is methyl. In other embodiments, both R⁸ and R⁹ are methyl.

In some different embodiments of Formula (I), R⁸ and R⁹, together with the nitrogen atom to which they are attached, form a 5, 6 or 7-membered heterocyclic ring. In some embodiments of the foregoing, R⁸ and R⁹, together with the nitrogen atom to which they are attached, form a 5-membered heterocyclic ring, for example a pyrrolidinyl ring.

In various different embodiments, the lipid of Formula (I) has one of the structures set forth in Table 1 below.

TABLE 1
Representative Lipids of Formula (I).

| | | Prep. |
|---|---|---|
| No. | Structure | Method |
| I-1 | 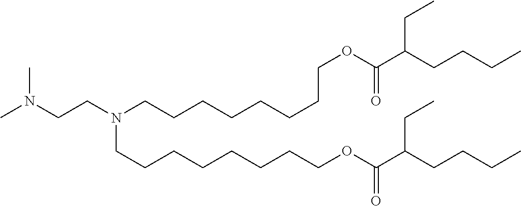 [SMILES: CCCCC(CC)C(=O)OCCCCCCCCN(CCCCCCCCOC(=O)C(CC)CCCC)CCN(C)C] | B |
| I-2 | 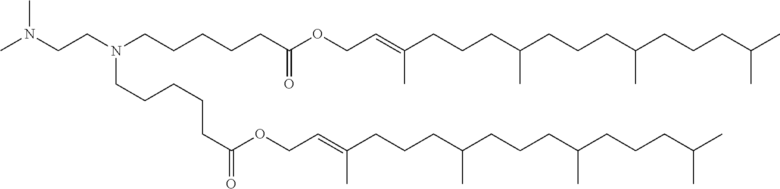 [SMILES: C/C(=C\COC(=O)CCCCCN(CCCCCC(=O)OC/C=C(\C)CCCC(C)CCCC(C)CCCC(C)C)CCN(C)C)CCCC(C)CCCC(C)CCCC(C)C] | A |
| I-3 | 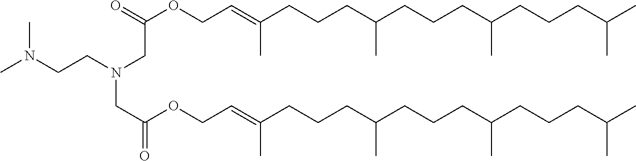 [SMILES: C/C(=C\COC(=O)CN(CCN(C)C)CC(=O)OC/C=C(\C)CCCC(C)CCCC(C)CCCC(C)C)CCCC(C)CCCC(C)CCCC(C)C] | A |
| I-4 | 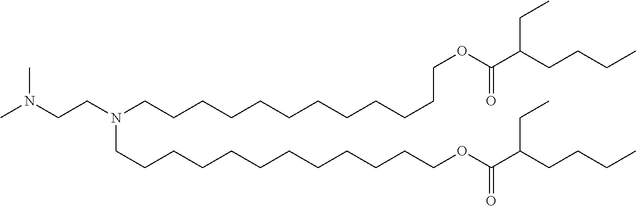 [SMILES: CCCCC(CC)C(=O)OCCCCCCCCCCCCN(CCCCCCCCCCCCOC(=O)C(CC)CCCC)CCN(C)C] | B |
| I-5 | 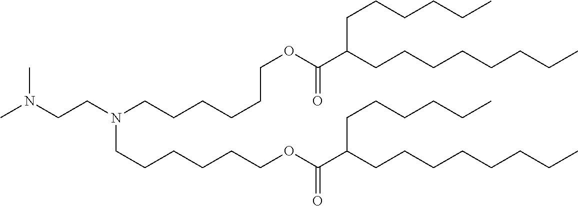 [SMILES: CCCCCCCCC(CCCCCC)C(=O)OCCCCCCN(CCCCCCOC(=O)C(CCCCCC)CCCCCCCC)CCN(C)C] | B |
| I-6 | 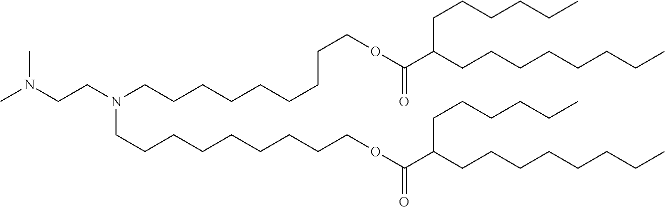 [SMILES: CCCCCCCCC(CCCCCC)C(=O)OCCCCCCCCCN(CCCCCCCCCOC(=O)C(CCCCCC)CCCCCCCC)CCN(C)C] | B |
| I-7 | 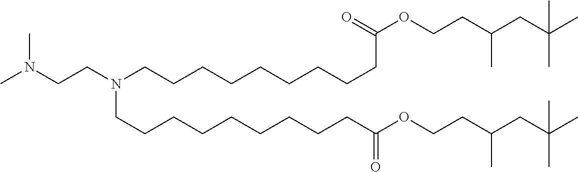 [SMILES: CC(CCOC(=O)CCCCCCCCCN(CCCCCCCCCC(=O)OCCC(C)CC(C)(C)C)CCN(C)C)CC(C)(C)C] | A |
| I-8 | 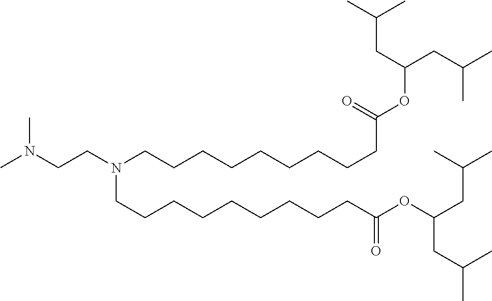 [SMILES: CC(C)CC(CC(C)C)OC(=O)CCCCCCCCCN(CCCCCCCCCC(=O)OC(CC(C)C)CC(C)C)CCN(C)C] | A |
| I-9 | 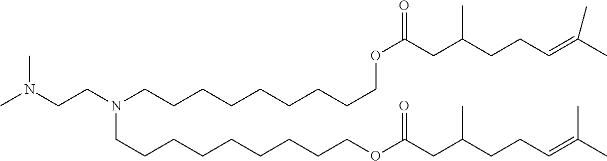 [SMILES: CC(C)=CCCC(C)CC(=O)OCCCCCCCCCN(CCCCCCCCCOC(=O)CC(C)CCC=C(C)C)CCN(C)C] | B |
| I-10 | 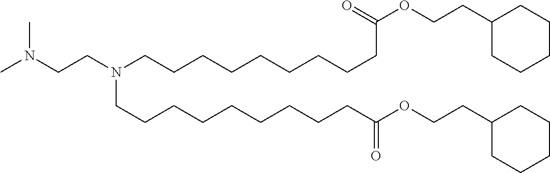 [SMILES: CN(C)CCN(CCCCCCCCCC(=O)OCCC1CCCCC1)CCCCCCCCCC(=O)OCCC1CCCCC1] | A |
| I-11 | 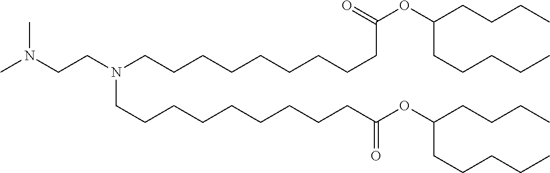 [SMILES: CCCCCC(CCCC)OC(=O)CCCCCCCCCN(CCCCCCCCCC(=O)OC(CCCC)CCCCC)CCN(C)C] | A |
| I-12 | 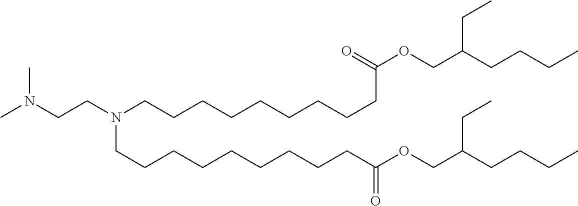 [SMILES: CCCCC(CC)COC(=O)CCCCCCCCCN(CCCCCCCCCC(=O)OCC(CC)CCCC)CCN(C)C] | A |
| I-13 | 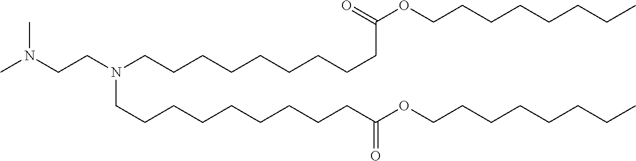 [SMILES: CCCCCCCCOC(=O)CCCCCCCCCN(CCCCCCCCCC(=O)OCCCCCCCC)CCN(C)C] | A |
| I-14 | 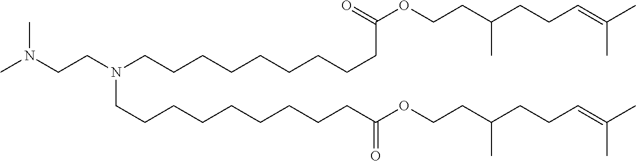 [SMILES: CC(C)=CCCC(C)CCOC(=O)CCCCCCCCCN(CCCCCCCCCC(=O)OCCC(C)CCC=C(C)C)CCN(C)C] | A |
| I-15 | 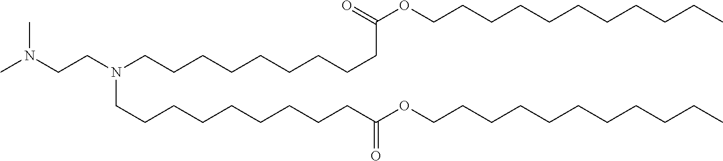 [SMILES: CCCCCCCCCCCOC(=O)CCCCCCCCCN(CCCCCCCCCC(=O)OCCCCCCCCCCC)CCN(C)C] | A |
| I-16 | 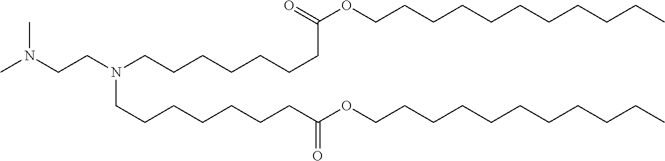 [SMILES: CCCCCCCCCCCOC(=O)CCCCCCCN(CCCCCCCC(=O)OCCCCCCCCCCC)CCN(C)C] | A |
| I-17 | 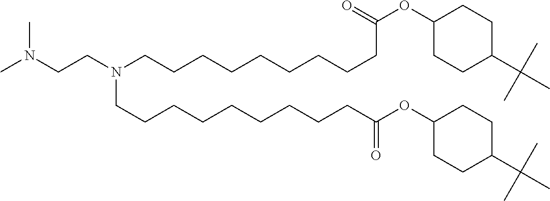 [SMILES: CN(C)CCN(CCCCCCCCCC(=O)OC1CCC(C(C)(C)C)CC1)CCCCCCCCCC(=O)OC1CCC(C(C)(C)C)CC1] | A |
| I-18 | 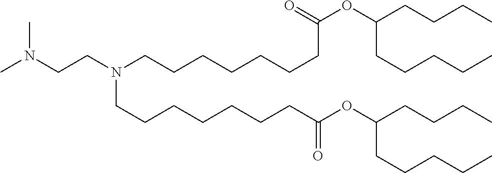 [SMILES: CCCCCC(CCCC)OC(=O)CCCCCCCN(CCCCCCCC(=O)OC(CCCC)CCCCC)CCN(C)C] | A |
| I-19 | 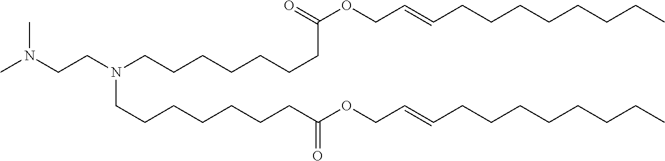 [SMILES: CCCCCCCC/C=C/COC(=O)CCCCCCCN(CCCCCCCC(=O)OC/C=C/CCCCCCCC)CCN(C)C] | A |
| I-20 | 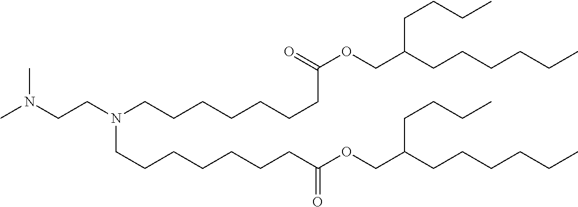 [SMILES: CCCCCCC(CCCC)COC(=O)CCCCCCCN(CCCCCCCC(=O)OCC(CCCC)CCCCCC)CCN(C)C] | A |
| I-21 | 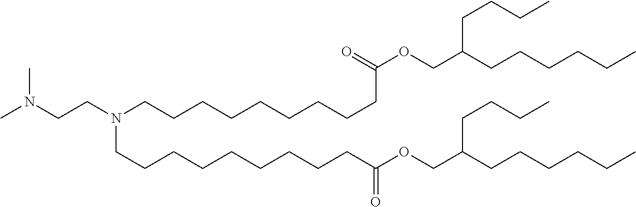 [SMILES: CCCCCCC(CCCC)COC(=O)CCCCCCCCCN(CCCCCCCCCC(=O)OCC(CCCC)CCCCCC)CCN(C)C] | A |
| I-22 | 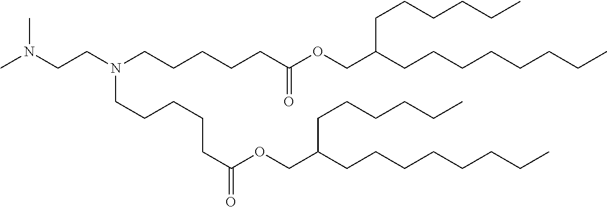 [SMILES: CCCCCCCCC(CCCCCC)COC(=O)CCCCCN(CCCCCC(=O)OCC(CCCCCC)CCCCCCCC)CCN(C)C] | A |
| I-23 | 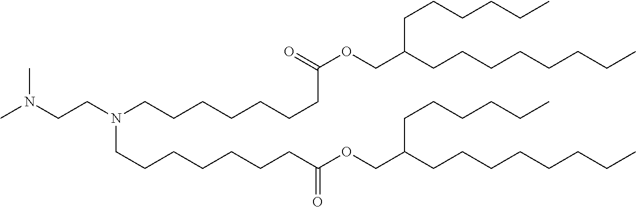 [SMILES: CCCCCCCCC(CCCCCC)COC(=O)CCCCCCCN(CCCCCCCC(=O)OCC(CCCCCC)CCCCCCCC)CCN(C)C] | A |
| I-24 | 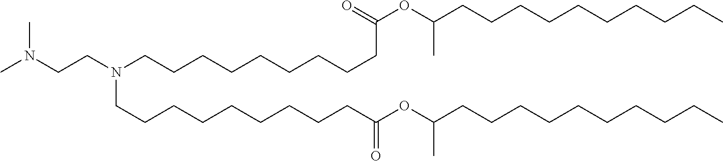 [SMILES: CCCCCCCCCCC(C)OC(=O)CCCCCCCCCN(CCCCCCCCCC(=O)OC(C)CCCCCCCCCC)CCN(C)C] | A |
| I-25 | 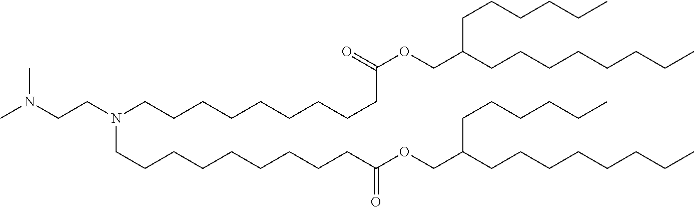 [SMILES: CCCCCCCCC(CCCCCC)COC(=O)CCCCCCCCCN(CCCCCCCCCC(=O)OCC(CCCCCC)CCCCCCCC)CCN(C)C] | A |
| I-26 | 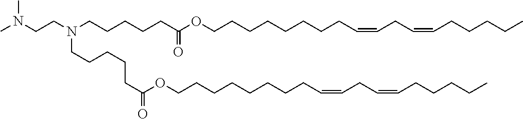 [SMILES: CCCCC/C=C\C/C=C\CCCCCCCCOC(=O)CCCCCN(CCCCCC(=O)OCCCCCCCC/C=C\C/C=C\CCCCC)CCN(C)C] | A |
| I-27 | 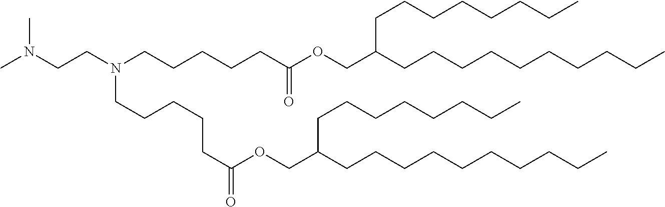 [SMILES: CCCCCCCCCCC(CCCCCCCC)COC(=O)CCCCCN(CCCCCC(=O)OCC(CCCCCCCC)CCCCCCCCCC)CCN(C)C] | A |
| I-28 | 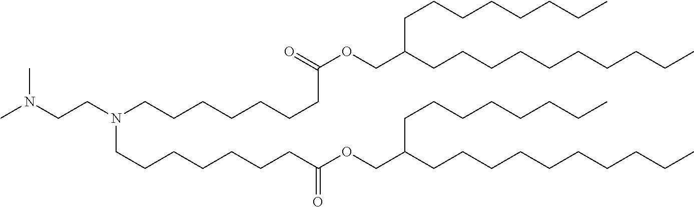 [SMILES: CCCCCCCCCCC(CCCCCCCC)COC(=O)CCCCCCCN(CCCCCCCC(=O)OCC(CCCCCCCC)CCCCCCCCCC)CCN(C)C] | A |
| I-29 | 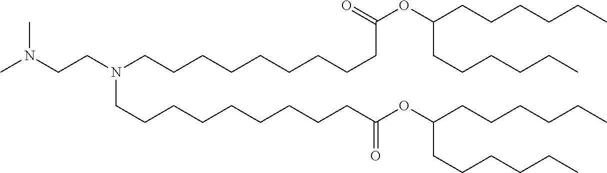 [SMILES: CCCCCCC(CCCCCC)OC(=O)CCCCCCCCCN(CCCCCCCCCC(=O)OC(CCCCCC)CCCCCC)CCN(C)C] | A |
| I-30 | 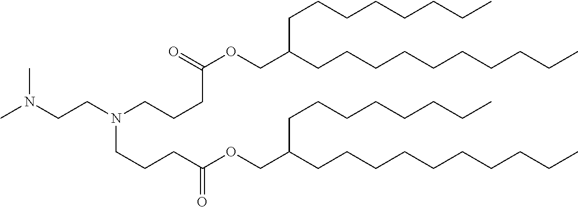 [SMILES: CCCCCCCCCCC(CCCCCCCC)COC(=O)CCCN(CCCC(=O)OCC(CCCCCCCC)CCCCCCCCCC)CCN(C)C] | A |
| I-31 | 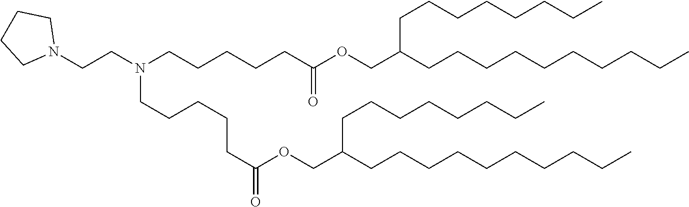 [SMILES: CCCCCCCCCCC(CCCCCCCC)COC(=O)CCCCCN(CCCCCC(=O)OCC(CCCCCCCC)CCCCCCCCCC)CCN1CCCC1] | C |
| I-32 | 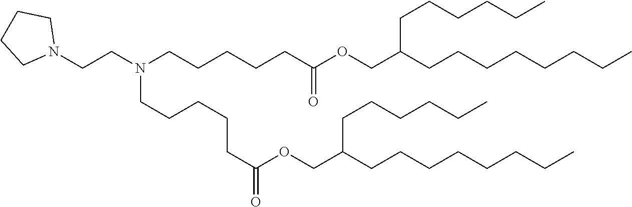 [SMILES: CCCCCCCCC(CCCCCC)COC(=O)CCCCCN(CCCCCC(=O)OCC(CCCCCC)CCCCCCCC)CCN1CCCC1] | C |
| I-33 | 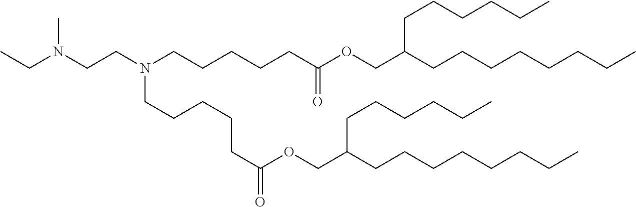 [SMILES: CCCCCCCCC(CCCCCC)COC(=O)CCCCCN(CCCCCC(=O)OCC(CCCCCC)CCCCCCCC)CCN(C)CC] | C |
| I-34 | 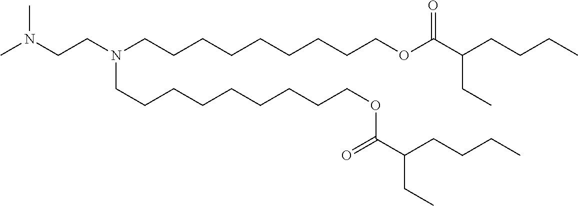 [SMILES: CCCCC(CC)C(=O)OCCCCCCCCCN(CCCCCCCCCOC(=O)C(CC)CCCC)CCN(C)C] | B |
| I-35 | 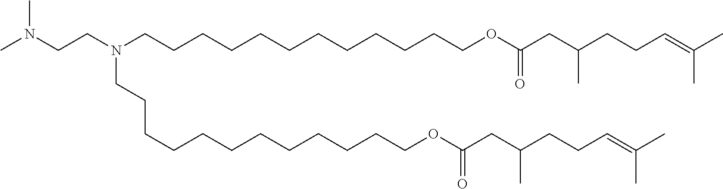 [SMILES: CC(C)=CCCC(C)CC(=O)OCCCCCCCCCCCCN(CCCCCCCCCCCCOC(=O)CC(C)CCC=C(C)C)CCN(C)C] | B |
| I-36 | 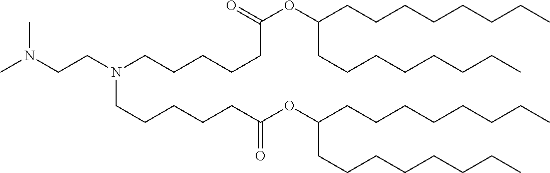 [SMILES: CCCCCCCCC(CCCCCCCC)OC(=O)CCCCCN(CCCCCC(=O)OC(CCCCCCCC)CCCCCCCC)CCN(C)C] | C |
| I-37 | 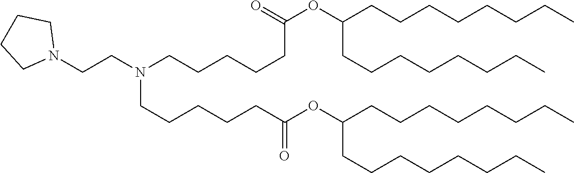 [SMILES: CCCCCCCCC(CCCCCCCC)OC(=O)CCCCCN(CCCCCC(=O)OC(CCCCCCCC)CCCCCCCC)CCN1CCCC1] | C |
| I-38 | 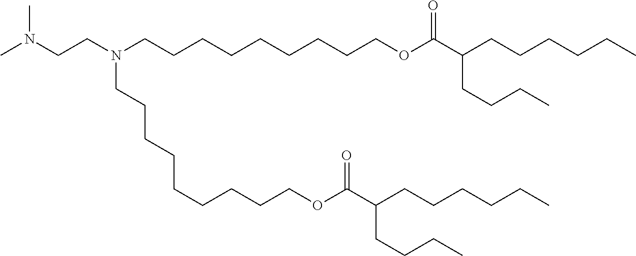 [SMILES: CCCCCCC(CCCC)C(=O)OCCCCCCCCCN(CCCCCCCCCOC(=O)C(CCCC)CCCCCC)CCN(C)C] | B |
| I-39 | 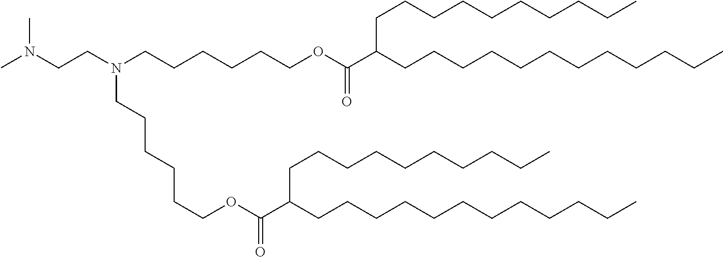 [SMILES: CCCCCCCCCCCCC(CCCCCCCCCC)C(=O)OCCCCCCN(CCCCCCOC(=O)C(CCCCCCCCCC)CCCCCCCCCCCC)CCN(C)C] | B |
| I-40 | 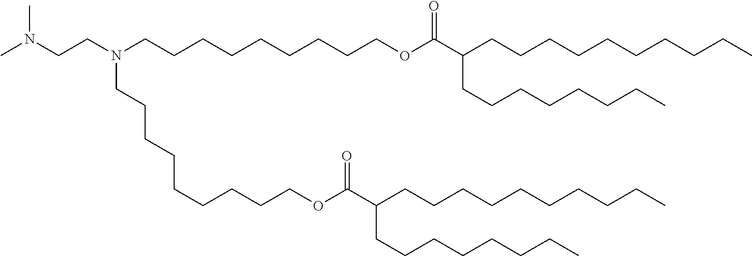 [SMILES: CCCCCCCCCCC(CCCCCCCC)C(=O)OCCCCCCCCCN(CCCCCCCCCOC(=O)C(CCCCCCCC)CCCCCCCCCC)CCN(C)C] | B |
| I-41 | 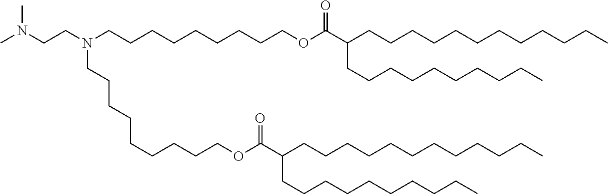 [SMILES: CCCCCCCCCCCCC(CCCCCCCCCC)C(=O)OCCCCCCCCCN(CCCCCCCCCOC(=O)C(CCCCCCCCCC)CCCCCCCCCCCC)CCN(C)C] | B |

In some embodiments, the LNPs comprise a lipid of Formula (I), a nucleoside-modified RNA and one or more excipients selected from neutral lipids, steroids and pegylated lipids. In some embodiments the lipid of Formula (I) is compound I-5. In some embodiments the lipid of Formula (I) is compound I-6.

In some other embodiments, the cationic lipid component of the LNPs has the structure of Formula (II):

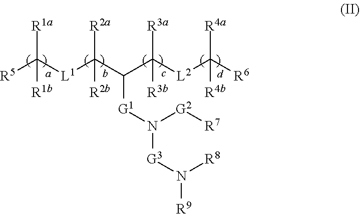

or a pharmaceutically acceptable salt, tautomer, prodrug or stereoisomer thereof, wherein:
L¹ and L² are each independently —O(C═O)—, —(C═O)O—, —C(═O)—, —O—,
—S(O)ₓ—, —S—S—, —C(═O)S—, —SC(═O)—, —NR^{a}C(═O)—, —C(═O)NR^{a}—, —NR^{a}C(═O)NR^{a}
—OC(═O)NR^{a}—, —NR^{a}C(═O)O—, or a direct bond;
G¹ is C₁-C₂ alkylene, —(C═O)—, —O(C═O)—, —SC(═O)—, —NR^{a}C(═O)— or a direct bond;
G² is —C(═O)—, —(C═O)O—, —C(═O)S—, —C(═O)NR^{a} or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either: (a) H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and
x is 0, 1 or 2.

In some embodiments of Formula (II), L¹ and L² are each independently
—O(C═O)—, —(C═O)O— or a direct bond. In other embodiments, G¹ and G² are each independently —(C═O)— or a direct bond. In some different embodiments, L¹ and L² are each independently —O(C═O)—, —(C═O)O— or a direct bond; and G¹ and G² are each independently —(C═O)— or a direct bond.

In some different embodiments of Formula (II), L¹ and L² are each independently —C(═O)—, —O—, —S(O)ₓ—, —S—S—, —C(═O)S—, —SC(═O)—, —NR^{a}—, —NR^{a}C(═O)—, —C(═O)NR^{a}—, —NR^{a}C(═O)NR^{a}, —OC(═O)NR^{a}—, —NR^{a}C(═O)O—, —NR^{a}S(O)ₓNR^{a}—, —NR^{a}S(O)ₓ— or —S(O)ₓNR^{a}—.

In other of the foregoing embodiments of Formula (II), the lipid compound has one of the following structures (IIA) or (IIB):

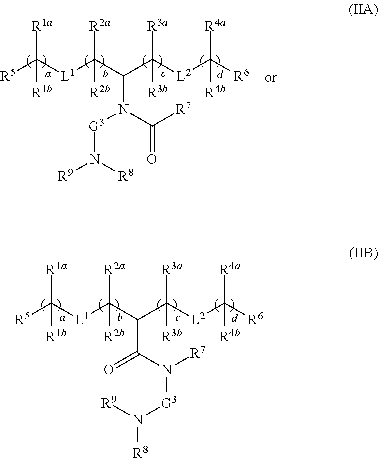

In some embodiments of Formula (II), the lipid compound has structure (IIA). In other embodiments, the lipid compound has structure (IIB).

In any of the foregoing embodiments of Formula (II), one of L¹ or L² is —O(C═O)—. For example, in some embodiments each of L¹ and L² are —O(C═O)—.

In some different embodiments of Formula (II), one of L¹ or L² is —(C═O)O—. For example, in some embodiments each of L¹ and L² is —(C═O)O—.

In different embodiments of Formula (II), one of L¹ or L² is a direct bond. As used herein, a “direct bond” means the group (e.g., L¹ or L²) is absent. For example, in some embodiments each of L¹ and L² is a direct bond.

In other different embodiments of Formula (II), for at least one occurrence of R^{1a} and R^{1b}, R^{1a} is H or C₁-C₁₂ alkyl, and R^{1b} together with the carbon atom to which it is bound is taken together with an adjacent R^{1b} and the carbon atom to which it is bound to form a carbon-carbon double bond.

In still other different embodiments of Formula (II), for at least one occurrence of R^{4a} and R^{4b}, R^{4a} is H or C₁-C₁₂ alkyl, and R^{4b} together with the carbon atom to which it is bound is taken together with an adjacent R^{4b} and the carbon atom to which it is bound to form a carbon-carbon double bond.

In more embodiments of Formula (II), for at least one occurrence of R^{2a} and R^{2b}, R^{2a} is H or C₁-C₁₂ alkyl, and R^{2b} together with the carbon atom to which it is bound is taken together with an adjacent R^{2b} and the carbon atom to which it is bound to form a carbon-carbon double bond.

In other different embodiments of Formula (II), for at least one occurrence of R^{3a} and R^{3b}, R^{3a} is H or C₁-C₁₂ alkyl, and R^{3b} together with the carbon atom to which it is bound is taken together with an adjacent R^{3b} and the carbon atom to which it is bound to form a carbon-carbon double bond.

In various other embodiments of Formula (II), the lipid compound has one of the following structures (IIC) or (IID).

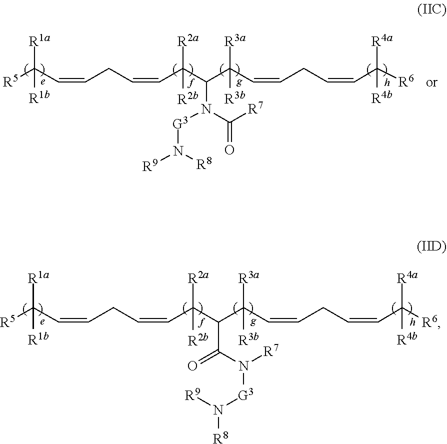

wherein e, f, g and h are each independently an integer from 1 to 12.

In some embodiments of Formula (II), the lipid compound has structure (IIC). In other embodiments, the lipid compound has structure (IID).

In various embodiments of structures (IIC) or (IID), e, f, g and h are each independently an integer from 4 to 10.

In some embodiments of Formula (II), a, b, c and d are each independently an integer from 2 to 12 or an integer from 4 to 12. In other embodiments, a, b, c and d are each independently an integer from 8 to 12 or 5 to 9. In some embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some embodiments of Formula (II), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some embodiments of Formula (II), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some embodiments of Formula (II), d is 0. In some embodiments, d is 1. In other embodiments, d is 2. In more embodiments, d is 3. In yet other embodiments, d is 4. In some embodiments, d is 5. In other embodiments, d is 6. In more embodiments, d is 7. In yet other embodiments, d is 8. In some embodiments, d is 9. In other embodiments, d is 10. In more embodiments, d is 11. In yet other embodiments, d is 12. In some embodiments, d is 13. In other embodiments, d is 14. In more embodiments, d is 15. In yet other embodiments, d is 16.

In some embodiments of Formula (II), e is 1. In other embodiments, e is 2. In more embodiments, e is 3. In yet other embodiments, e is 4. In some embodiments, e is 5. In other embodiments, e is 6. In more embodiments, e is 7. In yet other embodiments, e is 8. In some embodiments, e is 9. In other embodiments, e is 10. In more embodiments, e is 11. In yet other embodiments, e is 12.

In some embodiments of Formula (II), f is 1. In other embodiments, f is 2. In more embodiments, f is 3. In yet other embodiments, f is 4. In some embodiments, f is 5. In other embodiments, f is 6. In more embodiments, f is 7. In yet other embodiments, f is 8. In some embodiments, f is 9. In other embodiments, f is 10. In more embodiments, f is 11. In yet other embodiments, f is 12.

In some embodiments of Formula (II), g is 1. In other embodiments, g is 2. In more embodiments, g is 3. In yet other embodiments, g is 4. In some embodiments, g is 5. In other embodiments, g is 6. In more embodiments, g is 7. In yet other embodiments, g is 8. In some embodiments, g is 9. In other embodiments, g is 10. In more embodiments, g is 11. In yet other embodiments, g is 12.

In some embodiments of Formula (II), h is 1. In other embodiments, e is 2. In more embodiments, h is 3. In yet other embodiments, h is 4. In some embodiments, e is 5. In other embodiments, h is 6. In more embodiments, h is 7. In yet other embodiments, h is 8. In some embodiments, h is 9. In other embodiments, h is 10. In more embodiments, h is 11. In yet other embodiments, h is 12.

In some other various embodiments of Formula (II), a and d are the same. In some other embodiments, b and c are the same. In some other specific embodiments and a and d are the same and b and c are the same.

The sum of a and b and the sum of c and d of Formula (II) are factors which may be varied to obtain a lipid having the desired properties. In one embodiment, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a. b, c and d are selected such that the sum of a and b and the sum of c and d is 12 or greater.

The substituents at R^{1a}, R^{2a}, R^{3a} and R^{4a} of Formula (II) are not particularly limited. In some embodiments, at least one of R^{1a}, R^{2a}, R^{3a} and R^{4a} is H. In some embodiments R^{1a}, R^{2a}, R^{3a} and R^{4a} are H at each occurrence. In some other embodiments at least one of R^{1a}, R^{2a}, R^{3a} and R^{4a} is C₁-C₁₂ alkyl. In some other embodiments at least one of R^{1a}, R^{2a}, R^{3a} and R^{4a} is C₁-C₈ alkyl. In some other embodiments at least one of R^{1a}, R^{2a}, R^{3a} and R^{4a} is C₁-C₆ alkyl. In some of the foregoing embodiments, the C₁-C₈ alkyl is methyl, ethyl, n-propyl, iso-propyl, n-butyl, iso-butyl, tert-butyl, n-hexyl or n-octyl.

In some embodiments of Formula (II), R^{1a}, R^{1b}, R^{4a} and R^{4b} are C₁-C₁₂ alkyl at each occurrence.

In further embodiments of Formula (II), at least one of R^{1b}, R^{2b}, R^{3b} and R^{4b} is H or R^{1b}, R^{2b}, R^{3b} and R^{4b} are H at each occurrence.

In some embodiments of Formula (II), R^{1b} together with the carbon atom to which it is bound is taken together with an adjacent R^{1b} and the carbon atom to which it is bound to form a carbon-carbon double bond. In other embodiments of the foregoing R^{4b} together with the carbon atom to which it is bound is taken together with an adjacent R^{4b} and the carbon atom to which it is bound to form a carbon-carbon double bond.

The substituents at R⁵ and R⁶ of Formula (II) are not particularly limited in the foregoing embodiments. In some embodiments one of R⁵ or R⁶ is methyl. In other embodiments each of R⁵ or R⁶ is methyl.

The substituents at R⁷ of Formula (II) are not particularly limited in the foregoing embodiments. In some embodiments R⁷ is C₆-C₁₆ alkyl. In some other embodiments, R⁷ is C₆-C₉ alkyl. In some of these embodiments, R⁷ is substituted with —(C═O)OR^{b}, —O(C═O)R^{b}, —C(═O)R^{b}, —OR^{b}, —S(O)ₓR^{b}, —S—SR^{b}, —C(═O)SR^{b},
—SC(═O)R^{b}, —NR^{a}R^{b}, —NR^{a}C(═O)R^{b}, —C(═O)NR^{a}R^{b}, —NR^{a}C(═O)NR^{a}R^{b},
—OC(═O)NR^{a}R^{b}, —NR^{a}C(═O)OR^{b}, —NR^{a}S(O)ₓNR^{a}R^{b}, —NR^{a}S(O)ₓR or —S(O)ₓNR^{a}R^{b}, wherein: R^{a} is H or C₁-C₁₂ alkyl; R^{b} is C₁-C₁₅ alkyl; and x is 0, 1 or 2. For example, in some embodiments R⁷ is substituted with —(C═O)OR^{b} or —O(C═O)R^{b}.

In various of the foregoing embodiments of Formula (II), R^{b} is branched C₁-C₁₅ alkyl. For example, in some embodiments R^{b} has one of the following structures:

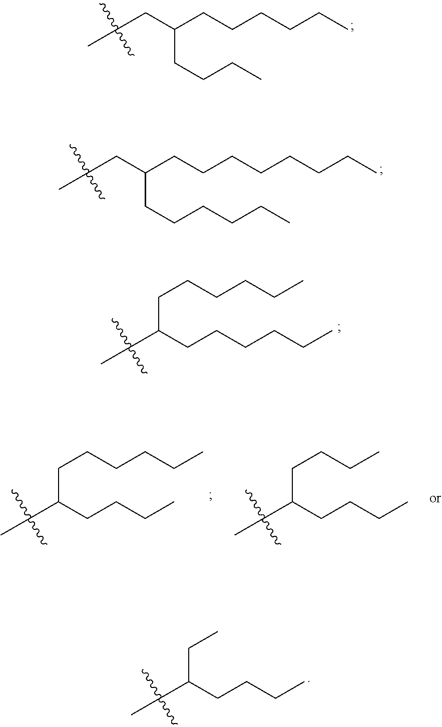

In some other of the foregoing embodiments of Formula (II), one of R⁸ or R⁹ is methyl. In other embodiments, both R⁸ and R⁹ are methyl.

In some different embodiments of Formula (II), R⁸ and R⁹, together with the nitrogen atom to which they are attached, form a 5, 6 or 7-membered heterocyclic ring. In some embodiments of the foregoing, R⁸ and R⁹, together with the nitrogen atom to which they are attached, form a 5-membered heterocyclic ring, for example a pyrrolidinyl ring. In some different embodiments of the foregoing, R⁸ and R⁹, together with the nitrogen atom to which they are attached, form a 6-membered heterocyclic ring, for example a piperazinyl ring.

In still other embodiments of the foregoing lipids of Formula (II), G³ is C₂-C₄ alkylene, for example C₃ alkylene.

In various different embodiments, the lipid compound has one of the structures set forth in Table 2 below.

TABLE 2
Representative Lipids of Formula (II).

| | | Prep. |
|---|---|---|
| No. | Structure | Method |
| II-1 | 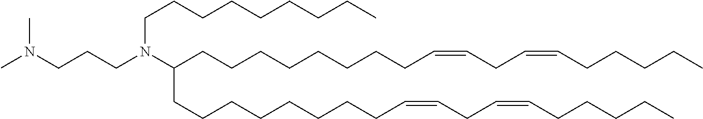 [SMILES: CCCCC/C=C\C/C=C\CCCCCCCCC(CCCCCCCC/C=C\C/C=C\CCCCC)N(CCCCCCCCC)CCCN(C)C] | D |
| II-2 | 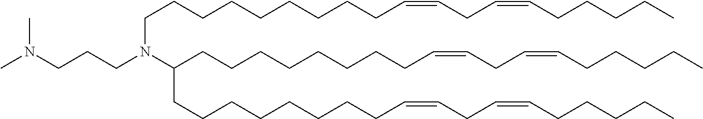 [SMILES: CCCCC/C=C\C/C=C\CCCCCCCCCN(CCCN(C)C)C(CCCCCCCC/C=C\C/C=C\CCCCC)CCCCCCCC/C=C\C/C=C\CCCCC] | D |
| II-3 | 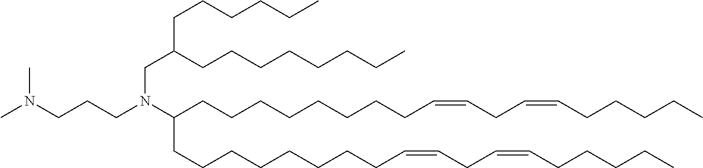 [SMILES: CCCCC/C=C\C/C=C\CCCCCCCCC(CCCCCCCC/C=C\C/C=C\CCCCC)N(CCCN(C)C)CC(CCCCCC)CCCCCCCC] | D |
| II-4 | 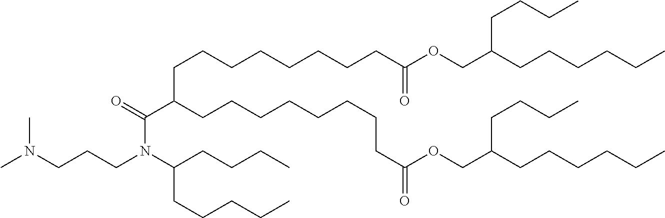 [SMILES: CCCCCCC(CCCC)COC(=O)CCCCCCCCC(CCCCCCCCC(=O)OCC(CCCC)CCCCCC)C(=O)N(CCCN(C)C)C(CCCC)CCCCC] | E |
| II-5 | 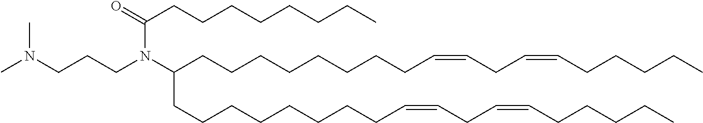 [SMILES: CCCCC/C=C\C/C=C\CCCCCCCCC(CCCCCCCC/C=C\C/C=C\CCCCC)N(CCCN(C)C)C(=O)CCCCCCCC] | D |
| II-6 | 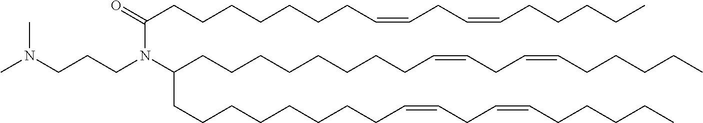 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC] | D |
| II-7 | 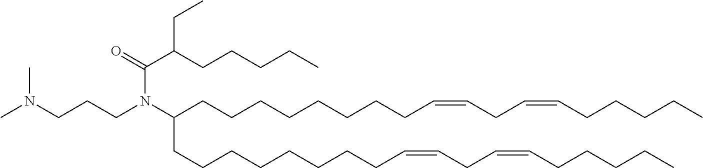 [SMILES: CCCCC/C=C\C/C=C\CCCCCCCCC(CCCCCCCC/C=C\C/C=C\CCCCC)N(CCCN(C)C)C(=O)C(CC)CCCCC] | D |
| II-8 | 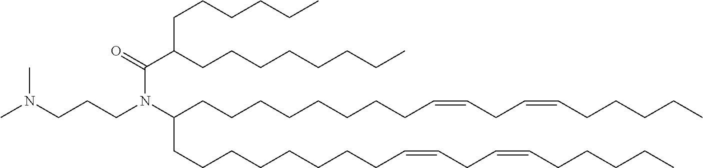 [SMILES: CCCCC/C=C\C/C=C\CCCCCCCCC(CCCCCCCC/C=C\C/C=C\CCCCC)N(CCCN(C)C)C(=O)C(CCCCCC)CCCCCCCC] | D |
| II-9 | 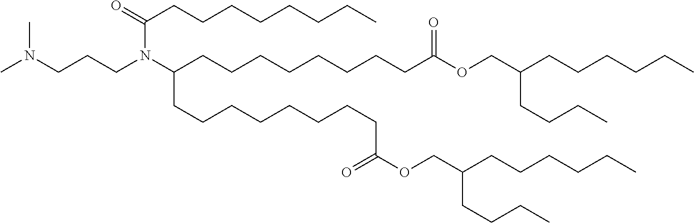 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC] | D |
| II-10 | 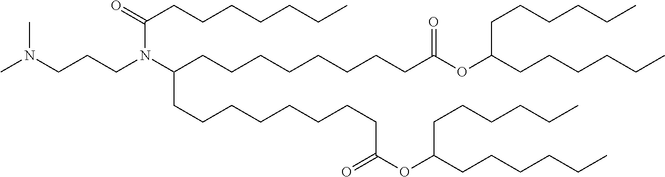 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] | D |
| II-11 | 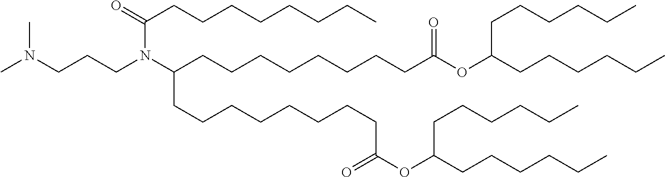 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC] | D |
| II-12 | 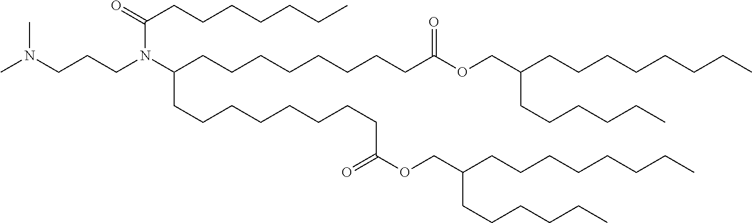 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC] | D |
| II-13 | 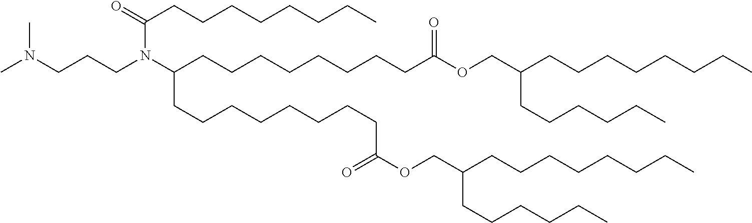 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC] | D |
| II-14 | 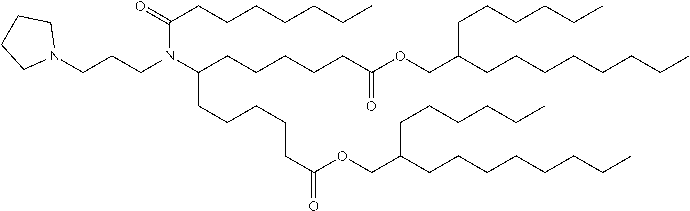 [SMILES: CCCCCCCCC(CCCCCC)COC(=O)CCCCCC(CCCCCC(=O)OCC(CCCCCC)CCCCCCCC)N(CCCN1CCCC1)C(=O)CCCCCCC] | D |
| II-15 | 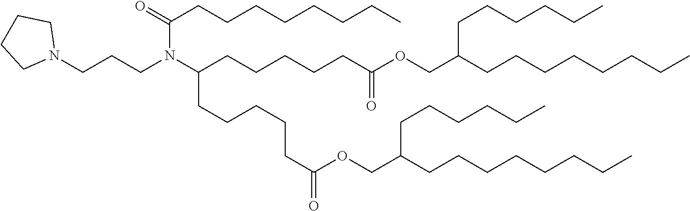 [SMILES: CCCCCCCCC(=O)N(CCCN1CCCC1)C(CCCCCC(=O)OCC(CCCCCC)CCCCCCCC)CCCCCC(=O)OCC(CCCCCC)CCCCCCCC] | D |
| II-16 | 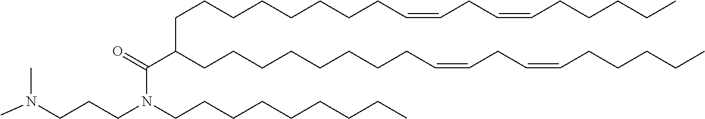 [SMILES: CCCCC/C=C\C/C=C\CCCCCCCCC(CCCCCCCC/C=C\C/C=C\CCCCC)C(=O)N(CCCCCCCCC)CCCN(C)C] | E |
| II-17 | 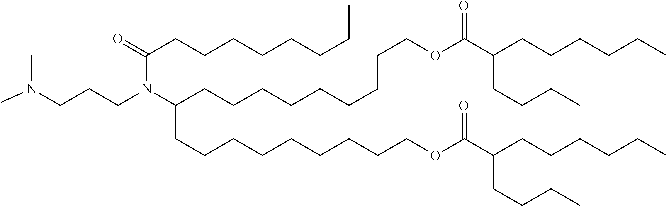 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC] | D |
| II-18 | 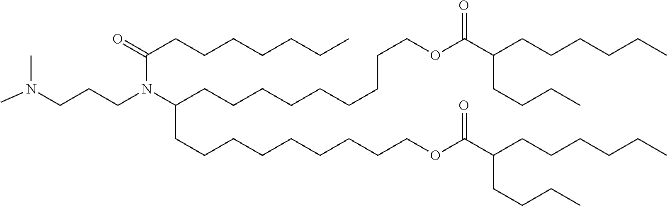 [SMILES: CCCCCCCC(=O)N(CCCN(C)C)C(CCCCCCCCCOC(=O)C(CCCC)CCCCCC)CCCCCCCCCOC(=O)C(CCCC)CCCCCC] | D |
| II-19 | 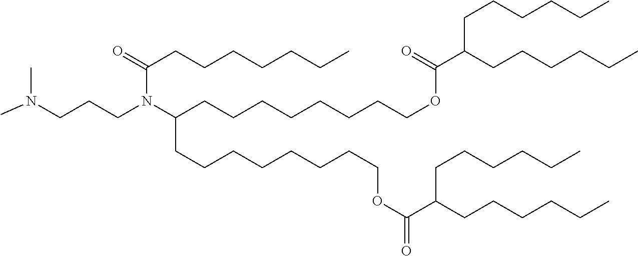 [SMILES: CCCCCCCC(=O)N(CCCN(C)C)C(CCCCCCCCOC(=O)C(CCCCCC)CCCCCC)CCCCCCCCOC(=O)C(CCCCCC)CCCCCC] | D |
| II-20 | 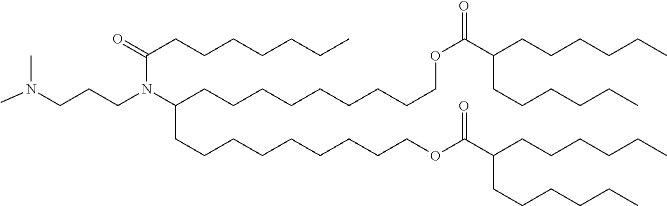 [SMILES: CCCCCCCC(=O)N(CCCN(C)C)C(CCCCCCCCCOC(=O)C(CCCCCC)CCCCCC)CCCCCCCCCOC(=O)C(CCCCCC)CCCCCC] | D |
| II-21 | 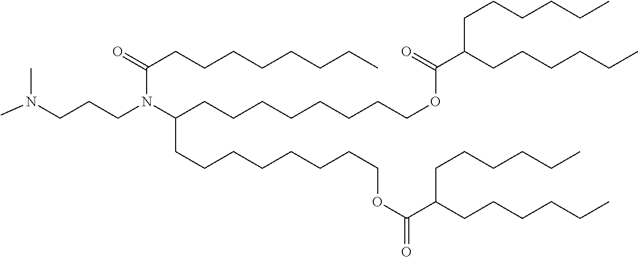 [SMILES: CCCCCCCCC(=O)N(CCCN(C)C)C(CCCCCCCCOC(=O)C(CCCCCC)CCCCCC)CCCCCCCCOC(=O)C(CCCCCC)CCCCCC] | D |
| II-22 | 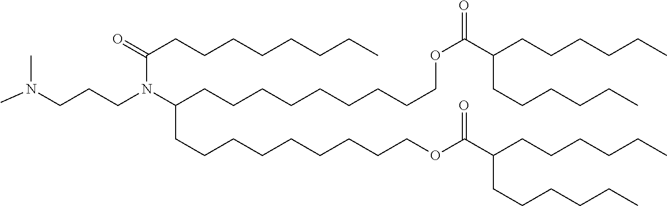 [SMILES: CCCCCCCCC(=O)N(CCCN(C)C)C(CCCCCCCCCOC(=O)C(CCCCCC)CCCCCC)CCCCCCCCCOC(=O)C(CCCCCC)CCCCCC] | D |
| II-23 | 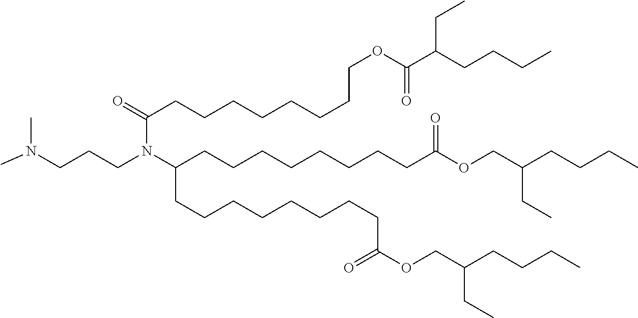 [SMILES: CCCCC(CC)COC(=O)CCCCCCCCC(CCCCCCCCC(=O)OCC(CC)CCCC)N(CCCN(C)C)C(=O)CCCCCCCCOC(=O)C(CC)CCCC] | D |
| II-24 | 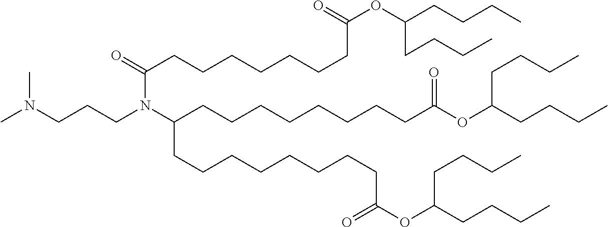 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC] | D |
| II-25 | 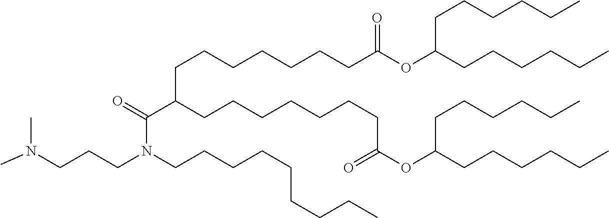 [SMILES: CCCCCCCCCN(CCCN(C)C)C(=O)C(CCCCCCCC(=O)OC(CCCCCC)CCCCCC)CCCCCCCC(=O)OC(CCCCCC)CCCCCC] | E |
| II-26 | 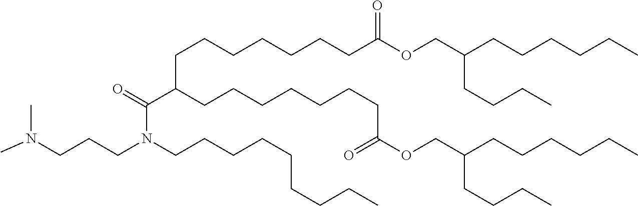 [SMILES: CCCCCCCCCN(CCCN(C)C)C(=O)C(CCCCCCCC(=O)OCC(CCCC)CCCCCC)CCCCCCCC(=O)OCC(CCCC)CCCCCC] | E |
| II-27 | 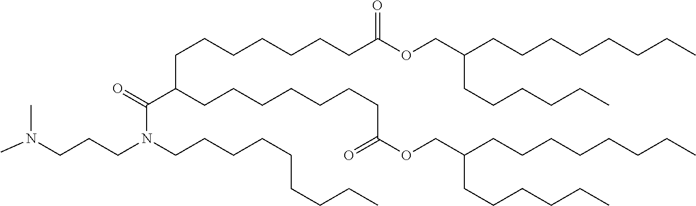 [SMILES: CCCCCCCCCN(CCCN(C)C)C(=O)C(CCCCCCCC(=O)OCC(CCCCCC)CCCCCCCC)CCCCCCCC(=O)OCC(CCCCCC)CCCCCCCC] | E |
| II-28 | 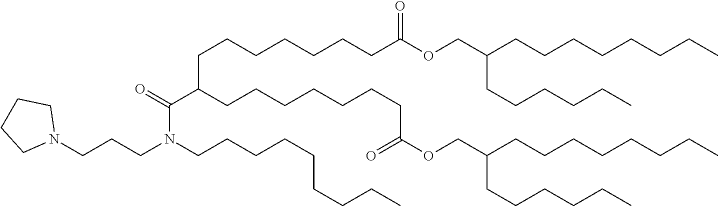 [SMILES: CCCCCCCCCN(CCCN1CCCC1)C(=O)C(CCCCCCCC(=O)OCC(CCCCCC)CCCCCCCC)CCCCCCCC(=O)OCC(CCCCCC)CCCCCCCC] | E |
| II-29 | 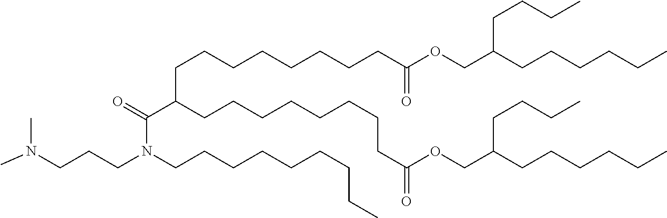 [SMILES: CCCCCCCCCN(CCCN(C)C)C(=O)C(CCCCCCCCC(=O)OCC(CCCC)CCCCCC)CCCCCCCCC(=O)OCC(CCCC)CCCCCC] | E |
| II-30 | 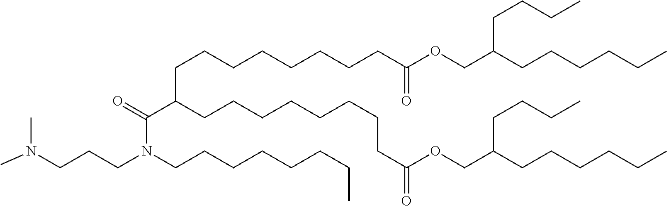 [SMILES: CCCCCCCCN(CCCN(C)C)C(=O)C(CCCCCCCCC(=O)OCC(CCCC)CCCCCC)CCCCCCCCC(=O)OCC(CCCC)CCCCCC] | E |
| II-31 | 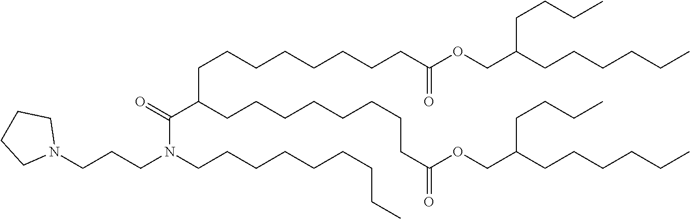 [SMILES: CCCCCCCCCN(CCCN1CCCC1)C(=O)C(CCCCCCCCC(=O)OCC(CCCC)CCCCCC)CCCCCCCCC(=O)OCC(CCCC)CCCCCC] | E |
| II-32 | 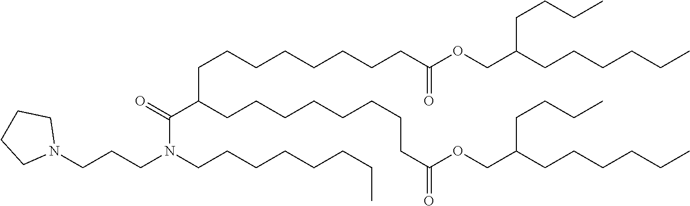 [SMILES: CCCCCCCCN(CCCN1CCCC1)C(=O)C(CCCCCCCCC(=O)OCC(CCCC)CCCCCC)CCCCCCCCC(=O)OCC(CCCC)CCCCCC] | E |
| II-33 | 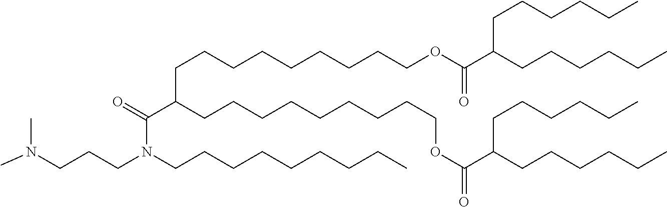 [SMILES: CCCCCCCCCN(CCCN(C)C)C(=O)C(CCCCCCCCCOC(=O)C(CCCCCC)CCCCCC)CCCCCCCCCOC(=O)C(CCCCCC)CCCCCC] | E |
| II-34 | 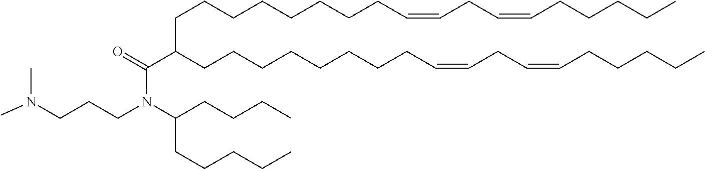 [SMILES: CCCCC/C=C\C/C=C\CCCCCCCCC(CCCCCCCC/C=C\C/C=C\CCCCC)C(=O)N(CCCN(C)C)C(CCCC)CCCCC] | E |
| II-35 | 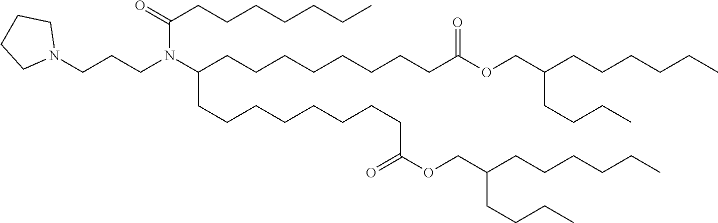 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC] | D |
| II-36 | 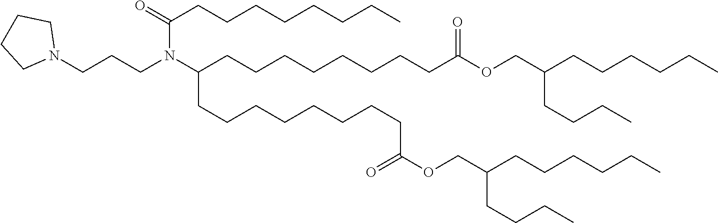 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC] | D |

In some embodiments, the LNPs comprise a lipid of Formula (II), a nucleoside-modified RNA and one or more excipient selected from neutral lipids, steroids and pegylated lipids. In some embodiments the lipid of Formula (II) is compound II-9. In some embodiments the lipid of Formula (II) is compound II-10. In some embodiments the lipid of Formula (II) is compound II-11. In some embodiments the lipid of Formula (II) is compound II-12. In some embodiments the lipid of Formula (II) is compound II-32.

In some other embodiments, the cationic lipid component of the LNPs has the structure of Formula (III):
(III)
or a pharmaceutically acceptable salt, tautomer, prodrug or stereoisomer thereof, wherein:
one of L¹ or L² is —O(C═O)—, —(C═O)O—, —C(═O)—, —O—, —S(O)ₓ—, —S—S—,
—C(═O)S—, SC(═O)—, —NR^{a}C(═O)—, —C(═O)NR^{a}—, NR^{a}C(═O)NR^{a}—, —OC(═O)NR^{a}—or
—NR^{a}C(═O)O—, and the other of L¹ or L² is —O(C═O)—, —(C═O)O—, —C(═O)—, —O—, —S(O)ₓ—,
—S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

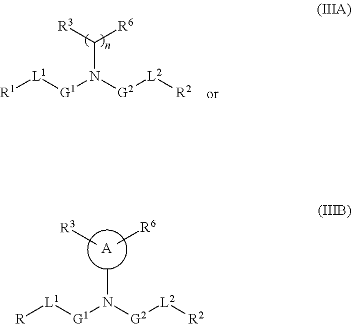

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

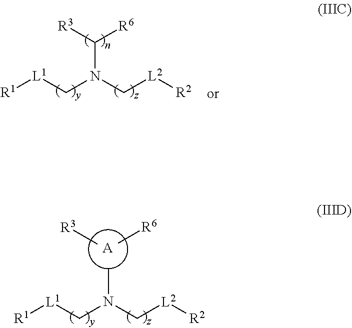

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures IIIE or (IIIF):

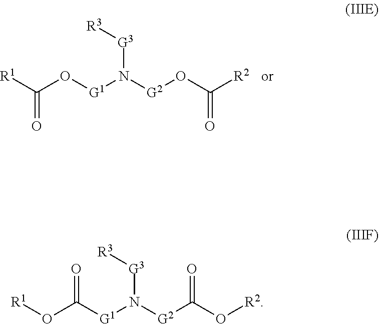

In some of the foregoing embodiments of Formula (III), the lipid has one of the following structures (IIIG), (IIIH), (IIII), or (IIIJ):

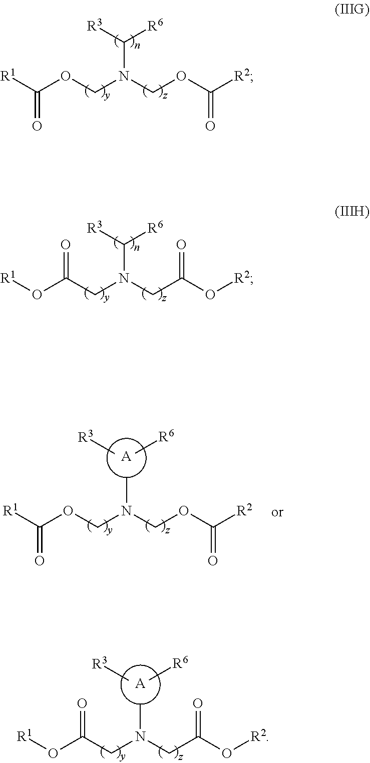

(IIII) (IIIJ)

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G3 is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

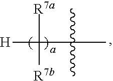

wherein:
R^{7a} and R^{7b} are, at each occurrence, independently H or C₁-C₁₂ alkyl; and
a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

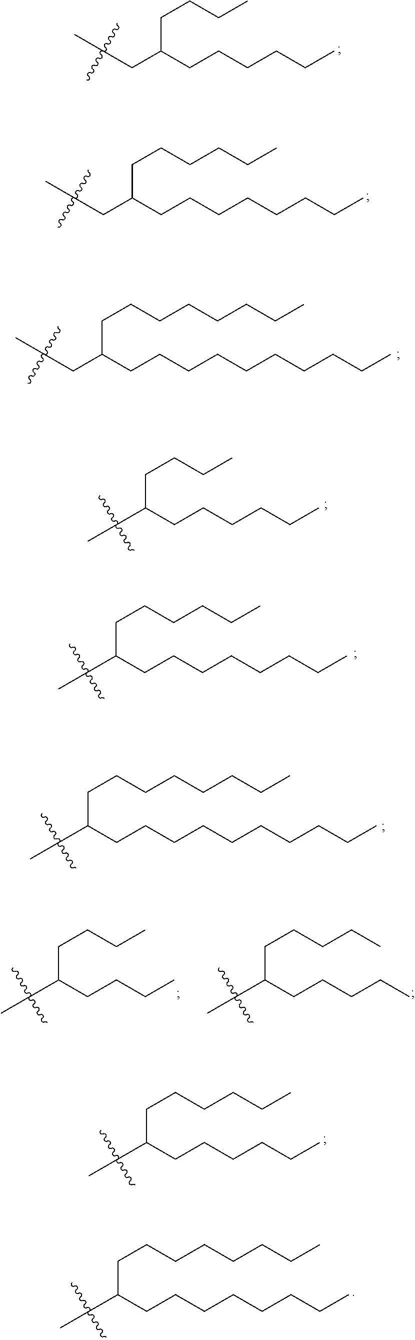

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the structures set forth in Table 3 below.

TABLE 3
Representative Compounds of Formula (III).

| | | Prep. |
|---|---|---|
| No. | Structure | Method |
| III-1 | 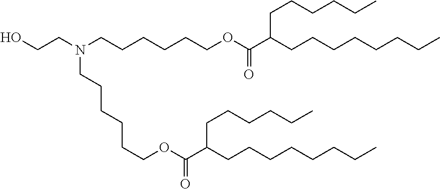 [SMILES: CCCCCCCCC(CCCCCC)C(=O)OCCCCCCN(CCO)CCCCCCOC(=O)C(CCCCCC)CCCCCCCC] | F |
| III-2 | 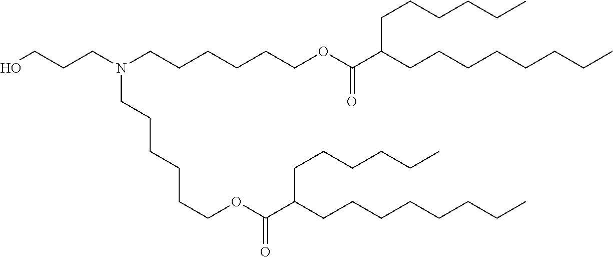 [SMILES: CCCCCCCCC(CCCCCC)C(=O)OCCCCCCN(CCCO)CCCCCCOC(=O)C(CCCCCC)CCCCCCCC] | F |
| III-3 | 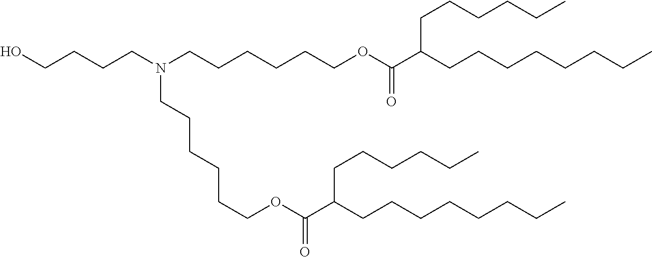 [SMILES: CCCCCCCCC(CCCCCC)C(=O)OCCCCCCN(CCCCO)CCCCCCOC(=O)C(CCCCCC)CCCCCCCC] | F |
| III-4 | 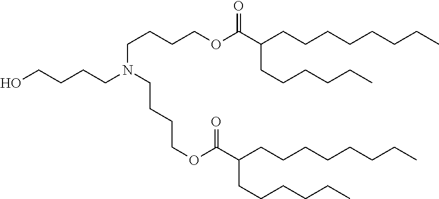 [SMILES: CCCCCCCCC(CCCCCC)C(=O)OCCCCN(CCCCO)CCCCOC(=O)C(CCCCCC)CCCCCCCC] | F |
| III-5 | 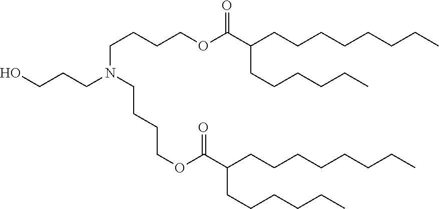 [SMILES: CCCCCCCCC(CCCCCC)C(=O)OCCCCN(CCCO)CCCCOC(=O)C(CCCCCC)CCCCCCCC] | F |
| III-6 | 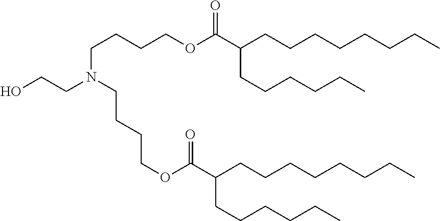 [SMILES: CCCCCCCCC(CCCCCC)C(=O)OCCCCN(CCO)CCCCOC(=O)C(CCCCCC)CCCCCCCC] | F |
| III-7 | 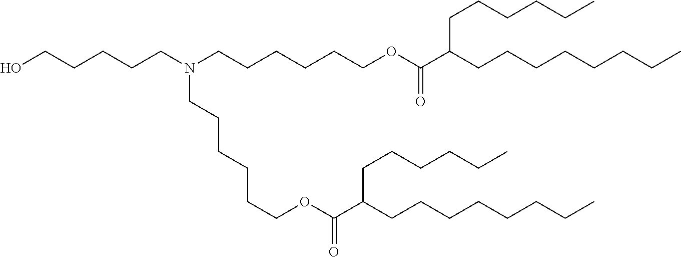 [SMILES: CCCCCCCCC(CCCCCC)C(=O)OCCCCCCN(CCCCCO)CCCCCCOC(=O)C(CCCCCC)CCCCCCCC] | F |
| III-8 | 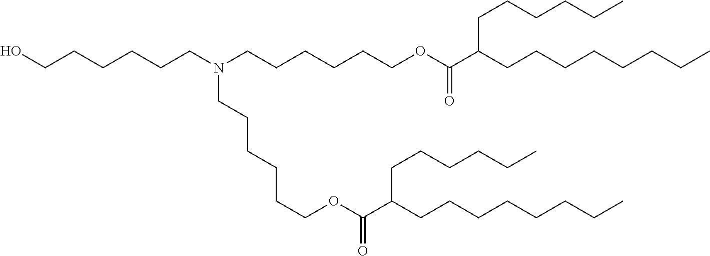 [SMILES: CCCCCCCCC(CCCCCC)C(=O)OCCCCCCN(CCCCCCO)CCCCCCOC(=O)C(CCCCCC)CCCCCCCC] | F |
| III-9 | 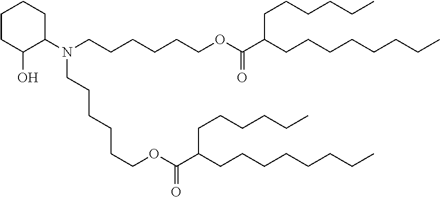 [SMILES: CCCCCCCCC(CCCCCC)C(=O)OCCCCCCN(CCCCCCOC(=O)C(CCCCCC)CCCCCCCC)C1CCCCC1O] | F |
| III-10 | 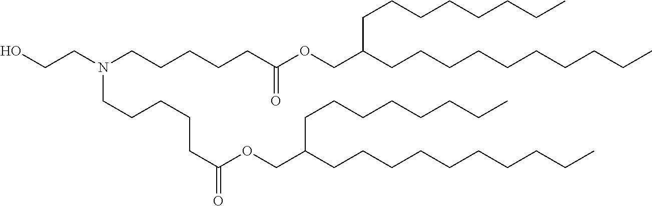 [SMILES: CCCCCCCCCCC(CCCCCCCC)COC(=O)CCCCCN(CCO)CCCCCC(=O)OCC(CCCCCCCC)CCCCCCCCCC] | F |
| III-11 | 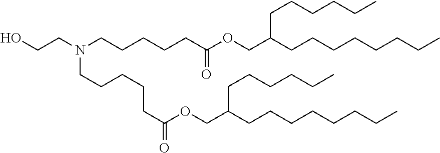 [SMILES: CCCCCCCCC(CCCCCC)COC(=O)CCCCCN(CCO)CCCCCC(=O)OCC(CCCCCC)CCCCCCCC] | F |
| III-12 | 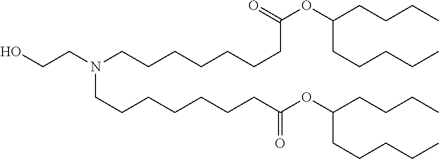 [SMILES: CCCCCC(CCCC)OC(=O)CCCCCCCN(CCO)CCCCCCCC(=O)OC(CCCC)CCCCC] | F |
| III-13 | 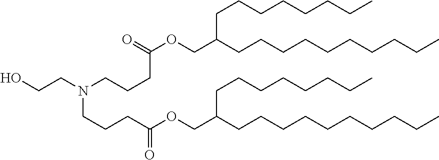 [SMILES: CCCCCCCCCCC(CCCCCCCC)COC(=O)CCCN(CCO)CCCC(=O)OCC(CCCCCCCC)CCCCCCCCCC] | F |
| III-14 | 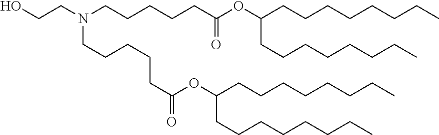 [SMILES: CCCCCCCCC(CCCCCCCC)OC(=O)CCCCCN(CCO)CCCCCC(=O)OC(CCCCCCCC)CCCCCCCC] | F |
| III-15 | 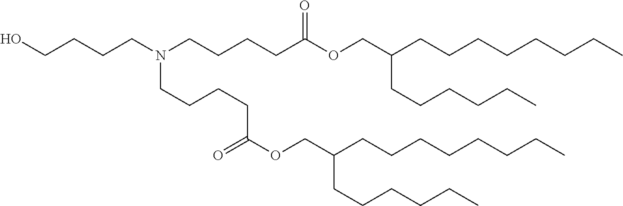 [SMILES: CCCCCCCCC(CCCCCC)COC(=O)CCCCN(CCCCO)CCCCC(=O)OCC(CCCCCC)CCCCCCCC] | F |
| III-16 | 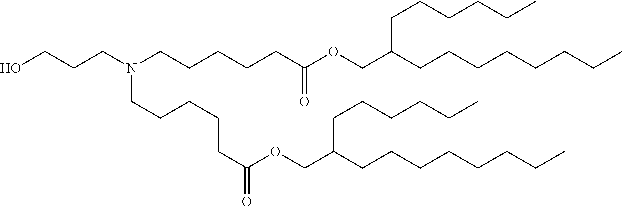 [SMILES: CCCCCCCCC(CCCCCC)COC(=O)CCCCCN(CCCO)CCCCCC(=O)OCC(CCCCCC)CCCCCCCC] | F |
| III-17 | 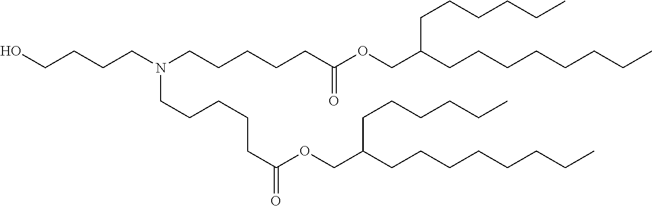 [SMILES: CCCCCCCCC(CCCCCC)COC(=O)CCCCCN(CCCCO)CCCCCC(=O)OCC(CCCCCC)CCCCCCCC] | F |
| III-18 | 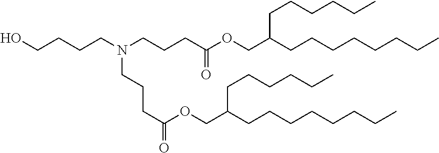 [SMILES: CCCCCCCCC(CCCCCC)COC(=O)CCCN(CCCCO)CCCC(=O)OCC(CCCCCC)CCCCCCCC] | F |
| III-19 | 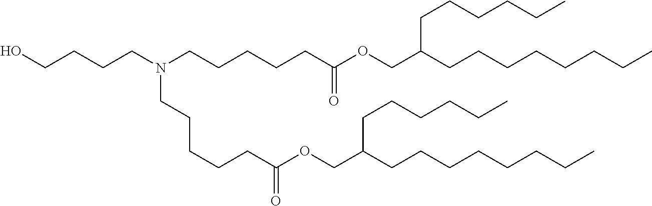 [SMILES: CCCCCCCCC(CCCCCC)COC(=O)CCCCCN(CCCCO)CCCCCC(=O)OCC(CCCCCC)CCCCCCCC] | F |
| III-20 | 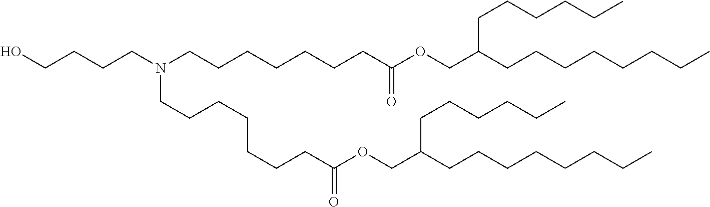 [SMILES: CCCCCCCCC(CCCCCC)COC(=O)CCCCCCCN(CCCCO)CCCCCCCC(=O)OCC(CCCCCC)CCCCCCCC] | F |
| III-21 | 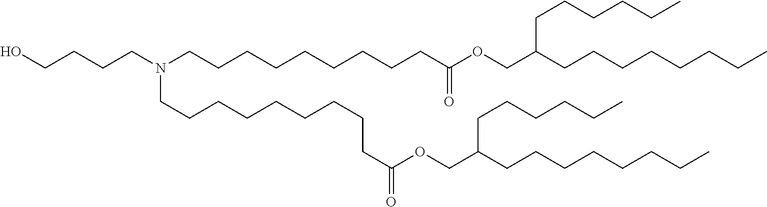 [SMILES: CCCCCCCCC(CCCCCC)COC(=O)CCCCCCCCCN(CCCCO)CCCCCCCCCC(=O)OCC(CCCCCC)CCCCCCCC] | F |
| III-22 | 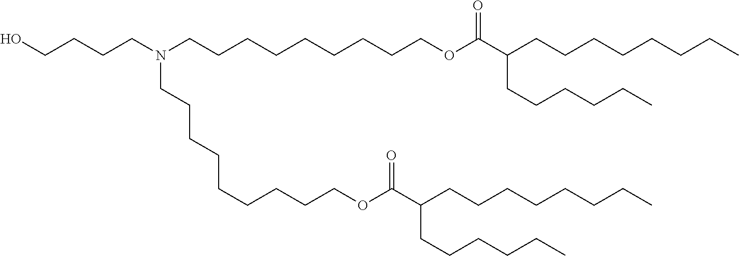 [SMILES: CCCCCCCCC(CCCCCC)C(=O)OCCCCCCCCCN(CCCCO)CCCCCCCCCOC(=O)C(CCCCCC)CCCCCCCC] | F |
| III-23 | 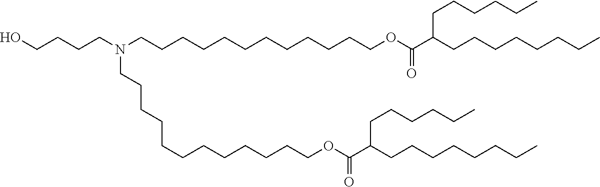 [SMILES: CCCCCCCCC(CCCCCC)C(=O)OCCCCCCCCCCCCN(CCCCO)CCCCCCCCCCCCOC(=O)C(CCCCCC)CCCCCCCC] | F |
| III-24 | 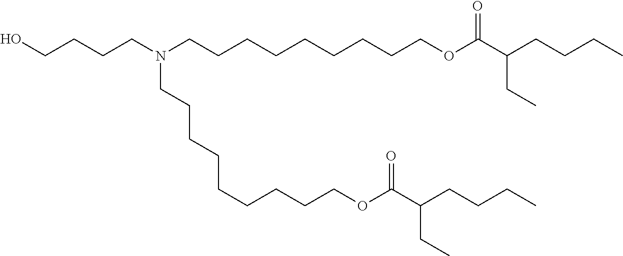 [SMILES: CCCCC(CC)C(=O)OCCCCCCCCCN(CCCCO)CCCCCCCCCOC(=O)C(CC)CCCC] | F |
| III-25 | 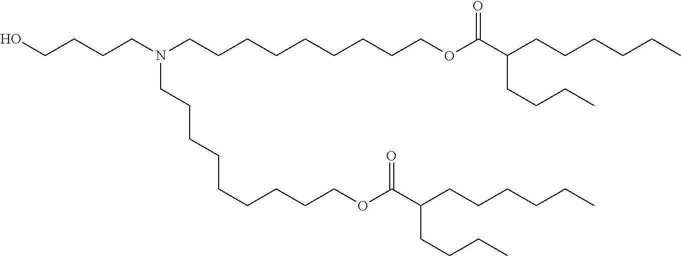 [SMILES: CCCCCCC(CCCC)C(=O)OCCCCCCCCCN(CCCCO)CCCCCCCCCOC(=O)C(CCCC)CCCCCC] | F |
| III-26 | 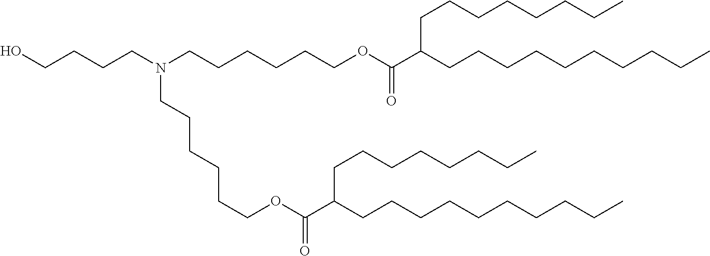 [SMILES: CCCCCCCCCCC(CCCCCCCC)C(=O)OCCCCCCN(CCCCO)CCCCCCOC(=O)C(CCCCCCCC)CCCCCCCCCC] | F |
| III-27 | 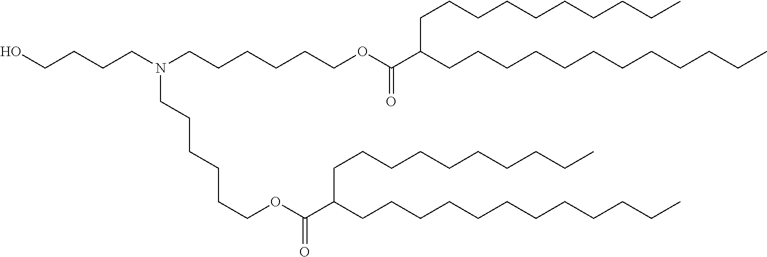 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] | F |
| III-28 | 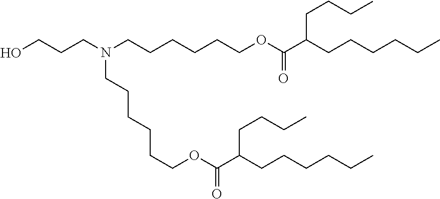 [SMILES: CCCCCCC(CCCC)C(=O)OCCCCCCN(CCCO)CCCCCCOC(=O)C(CCCC)CCCCCC] | F |
| III-29 | 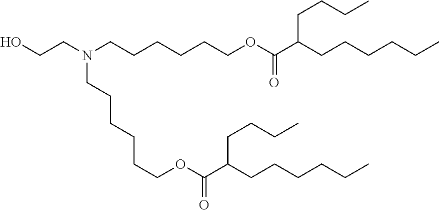 [SMILES: CCCCCCC(CCCC)C(=O)OCCCCCCN(CCO)CCCCCCOC(=O)C(CCCC)CCCCCC] | F |
| III-30 | 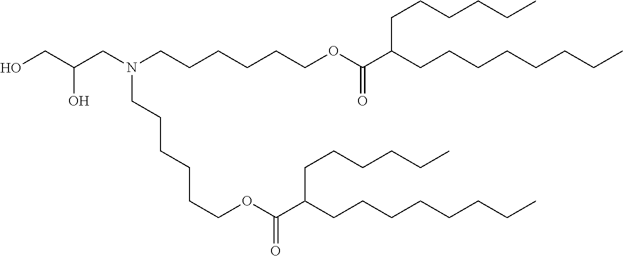 [SMILES: CCCCCCCCC(CCCCCC)C(=O)OCCCCCCN(CCCCCCOC(=O)C(CCCCCC)CCCCCCCC)CC(O)CO] | F |
| III-31 | 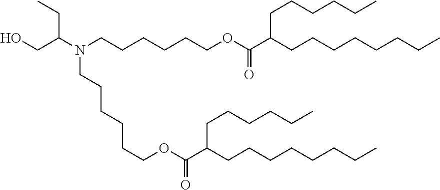 [SMILES: CCCCCCCCC(CCCCCC)C(=O)OCCCCCCN(CCCCCCOC(=O)C(CCCCCC)CCCCCCCC)C(CC)CO] | F |
| III-32 | 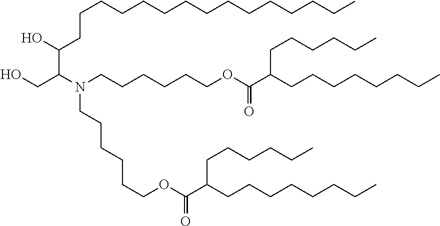 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] | F |
| III-33 | 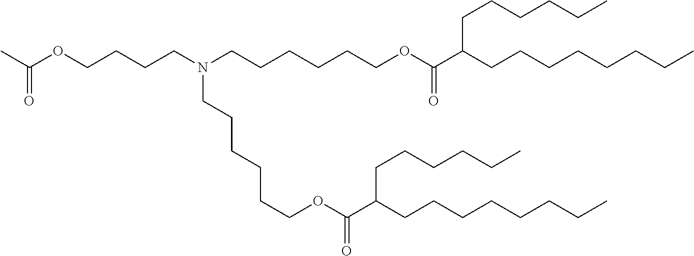 [SMILES: CCCCCCCCC(CCCCCC)C(=O)OCCCCCCN(CCCCCCOC(=O)C(CCCCCC)CCCCCCCC)CCCCOC(C)=O] | F |
| III-34 | 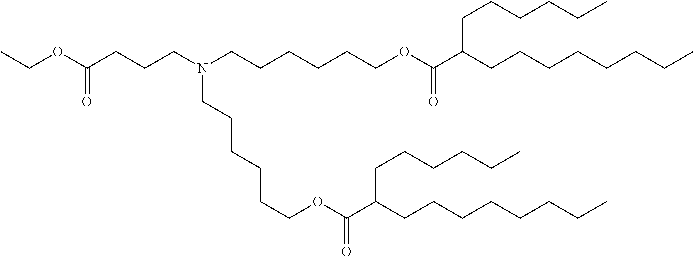 [SMILES: CCCCCCCCC(CCCCCC)C(=O)OCCCCCCN(CCCCCCOC(=O)C(CCCCCC)CCCCCCCC)CCCC(=O)OCC] | F |
| III-35 | 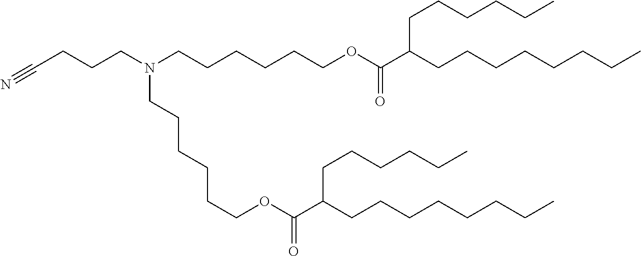 [SMILES: CCCCCCCCC(CCCCCC)C(=O)OCCCCCCN(CCCC#N)CCCCCCOC(=O)C(CCCCCC)CCCCCCCC] | F |
| III-36 | 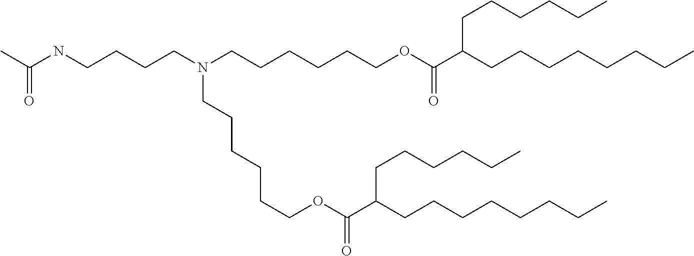 [SMILES: CCCCCCCCC(CCCCCC)C(=O)OCCCCCCN(CCCCCCOC(=O)C(CCCCCC)CCCCCCCC)CCCCNC(C)=O] | F |

In some embodiments, the LNPs comprise a lipid of Formula (III), a nucleoside-modified RNA and one or more excipient selected from neutral lipids, steroids and pegylated lipids. In some embodiments the lipid of Formula (III) is compound III-3. In some embodiments the lipid of Formula (III) is compound III-7.

In some embodiments, the cationic lipid is present in the LNP in an amount from about 30 to about 95 mole percent. In one embodiment, the cationic lipid is present in the LNP in an amount from about 30 to about 70 mole percent. In one embodiment, the cationic lipid is present in the LNP in an amount from about 40 to about 60 mole percent. In one embodiment, the cationic lipid is present in the LNP in an amount of about 50 mole percent. In one embodiment, the LNP comprises only cationic lipids.

In some embodiments, the LNP comprises one or more additional lipids which stabilize the formation of particles during their formation.

Suitable stabilizing lipids include neutral lipids and anionic lipids.

Exemplary anionic lipids include, but are not limited to, phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE). In one embodiment, the neutral lipid is 1,2-distearoyl-sn-glycero-3-phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid (e.g., lipid of Formula (I)) to the neutral lipid ranges from about 2:1 to about 8:1.

In various embodiments, the LNPs further comprise a steroid or steroid analogue. A “steroid” is a compound comprising the following carbon skeleton:

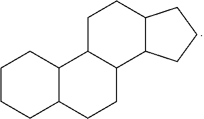 [SMILES: C1CCC2C(C1)CCC1C3CCCC3CCC21]

In some embodiments, the steroid or steroid analogue is cholesterol. In some of these embodiments, the molar ratio of the cationic lipid (e.g., lipid of Formula (I)) to cholesterol ranges from about 2:1 to 1:1.

In some embodiments, the LNP comprises glycolipids (e.g., monosialoganglioside GM₁). In some embodiments, the LNP comprises a sterol, such as cholesterol.

In some embodiments, the LNPs comprise a polymer conjugated lipid.

In some embodiments, the LNP comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)₂₀₀₀)carbamyl]-1,2-dimyristyloxlpropyl-3-amine (PEG-c-DMA). In one embodiment, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate. In various embodiments, the molar ratio of the cationic lipid to the pegylated lipid ranges from about 100:1 to about 25:1.

In some embodiments, the LNPs comprise a pegylated lipid having the following structure (IV):

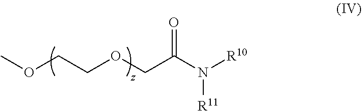

or a pharmaceutically acceptable salt, tautomer or stereoisomer thereof, wherein:
R¹⁰ and R¹¹ are each independently a straight or branched, saturated or unsaturated alkyl chain containing from 10 to 30 carbon atoms, wherein the alkyl chain is optionally interrupted by one or more ester bonds; and
z has mean value ranging from 30 to 60.

In some of the foregoing embodiments of the pegylated lipid (IV), R¹⁰ and R¹¹ are not both n-octadecyl when z is 42. In some other embodiments, R¹⁰ and R¹¹ are each independently a straight or branched, saturated or unsaturated alkyl chain containing from 10 to 18 carbon atoms. In some embodiments, R¹⁰ and R¹¹ are each independently a straight or branched, saturated or unsaturated alkyl chain containing from 12 to 16 carbon atoms. In some embodiments, R¹⁰ and R¹¹ are each independently a straight or branched, saturated or unsaturated alkyl chain containing 12 carbon atoms. In some embodiments, R¹⁰ and R¹¹ are each independently a straight or branched, saturated or unsaturated alkyl chain containing 14 carbon atoms. In other embodiments, R¹⁰ and R¹¹ are each independently a straight or branched, saturated or unsaturated alkyl chain containing 16 carbon atoms. In still more embodiments, R¹⁰ and R¹¹ are each independently a straight or branched, saturated or unsaturated alkyl chain containing 18 carbon atoms. In still other embodiments, R¹⁰ is a straight or branched, saturated or unsaturated alkyl chain containing 12 carbon atoms and R¹¹ is a straight or branched, saturated or unsaturated alkyl chain containing 14 carbon atoms.

In various embodiments, z spans a range that is selected such that the PEG portion of (II) has an average molecular weight of about 400 to about 6000 g/mol. In some embodiments, the average z is about 45.

In other embodiments, the pegylated lipid has one of the following structures:

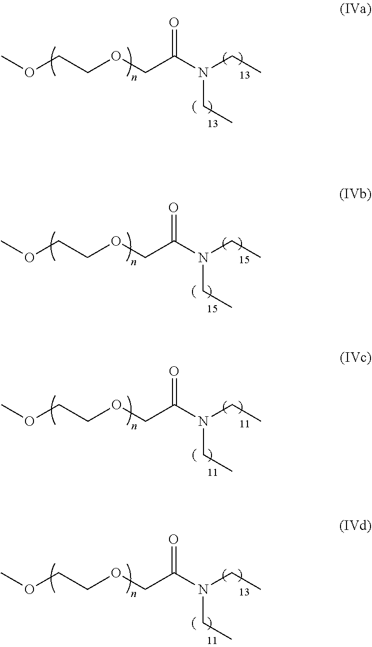

wherein n is an integer selected such that the average molecular weight of the pegylated lipid is about 2500 g/mol.

In some embodiments, the additional lipid is present in the LNP in an amount from about 1 to about 10 mole percent. In one embodiment, the additional lipid is present in the LNP in an amount from about 1 to about 5 mole percent. In one embodiment, the additional lipid is present in the LNP in about 1 mole percent or about 1.5 mole percent.

In some embodiments, the LNPs comprise a lipid of Formula (I), a nucleoside-modified RNA, a neutral lipid, a steroid and a pegylated lipid. In some embodiments the lipid of Formula (I) is compound I-6. In different embodiments, the neutral lipid is DSPC. In other embodiments, the steroid is cholesterol. In still different embodiments, the pegylated lipid is compound IVa.

In some embodiments, the LNP comprises one or more targeting moieties, which are capable of targeting the LNP to a cell or cell population. For example, in one embodiment, the targeting moiety is a ligand, which directs the LNP to a receptor found on a cell surface.

In some embodiments, the LNP comprises one or more internalization domains. For example, in one embodiment, the LNP comprises one or more domains, which bind to a cell to induce the internalization of the LNP. For example, in one embodiment, the one or more internalization domains bind to a receptor found on a cell surface to induce receptor-mediated uptake of the LNP. In some embodiments, the LNP is capable of binding a biomolecule in vivo, where the LNP-bound biomolecule can then be recognized by a cell-surface receptor to induce internalization. For example, in one embodiment, the LNP binds systemic ApoE, which leads to the uptake of the LNP and associated cargo.

Other exemplary LNPs and their manufacture are described in the art, for example in U.S. Patent Application Publication No. US20120276209, Semple et al., 2010, Nat Biotechnol., 28(2):172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer., 131(5): E781-90; Belliveau et al., 2012, Mol Ther nucleic Acids, 1: e37; Jayaraman et al., 2012, Angew Chem Int Ed Engl., 51(34): 8529-8533; Mui et al., 2013, Mol Ther Nucleic Acids. 2, e139; Maier et al., 2013, Mol Ther., 21(8): 1570-1578; and Tam et al., 2013, Nanomedicine, 9(5): 665-74, each of which are incorporated by reference in their entirety.

The following Reaction Schemes illustrate methods to make lipids of Formula (I), (II) or (III).

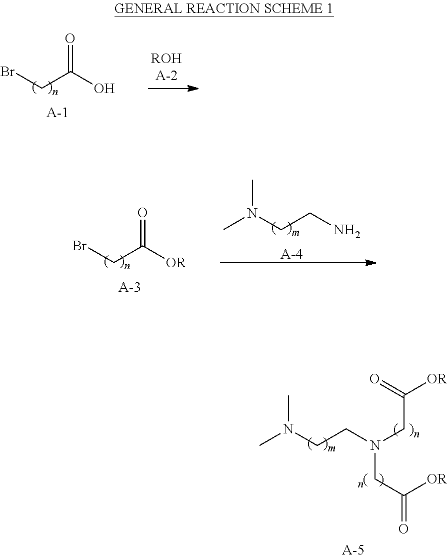

Embodiments of the lipid of Formula (I e.g., compound A-5) can be prepared according to General Reaction Scheme 1 (“Method A”), wherein R is a saturated or unsaturated C₁-C₂₄ alkyl or saturated or unsaturated cycloalkyl, m is 0 or 1 and n is an integer from 1 to 24. Referring to General Reaction Scheme 1, compounds of structure A-1 can be purchased from commercial sources or prepared according to methods familiar to one of ordinary skill in the art. A mixture of A-1, A-2 and DMAP is treated with DCC to give the bromide A-3. A mixture of the bromide A-3, a base (e.g., N,N-diisopropylethylamine) and the N,N-dimethyldiamine A-4 is heated at a temperature and time sufficient to produce A-5 after any necessarily workup and or purification step.

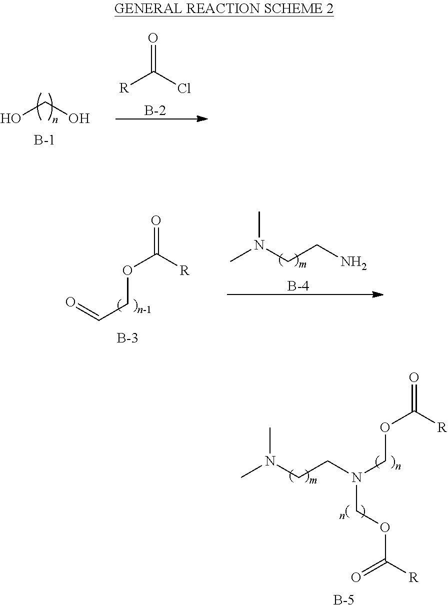

Other embodiments of the compound of Formula (I) (e.g., compound B-5) can be prepared according to General Reaction Scheme 2 (“Method B”), wherein R is a saturated or unsaturated C₁-C₂₄ alkyl or saturated or unsaturated cycloalkyl, m is 0 or 1 and n is an integer from 1 to 24. As shown in General Reaction Scheme 2, compounds of structure B-1 can be purchased from commercial sources or prepared according to methods familiar to one of ordinary skill in the art. A solution of B-1 (1 equivalent) is treated with acid chloride B-2 (1 equivalent) and a base (e.g., triethylamine). The crude product is treated with an oxidizing agent (e.g., pyridinum chlorochromate) and intermediate product B-3 is recovered. A solution of crude B-3, an acid (e.g., acetic acid), and N,N-dimethylaminoamine B-4 is then treated with a reducing agent (e.g., sodium triacetoxyborohydride) to obtain B-5 after any necessary work up and/or purification.

It should be noted that although starting materials A-1 and B-1 are depicted above as including only saturated methylene carbons, starting materials which include carbon-carbon double bonds may also be employed for preparation of compounds which include carbon-carbon double bonds.

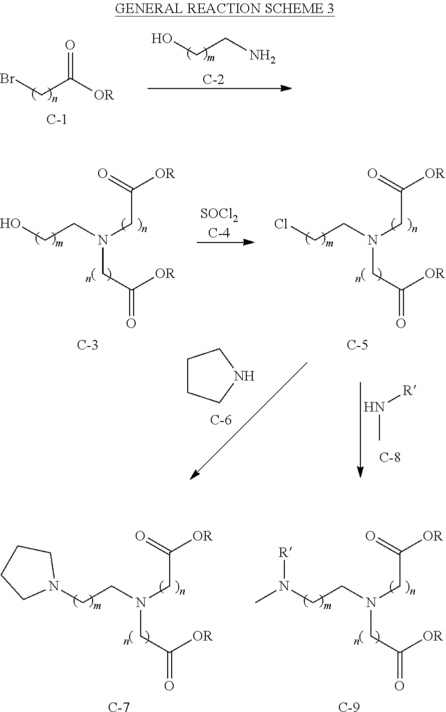

Different embodiments of the lipid of Formula (I) (e.g., compound C-7 or C9) can be prepared according to General Reaction Scheme 3 (“Method C”), wherein R is a saturated or unsaturated C₁-C₂₄ alkyl or saturated or unsaturated cycloalkyl, m is 0 or 1 and n is an integer from 1 to 24. Referring to General Reaction Scheme 3, compounds of structure C-1 can be purchased from commercial sources or prepared according to methods familiar to one of ordinary skill in the art.

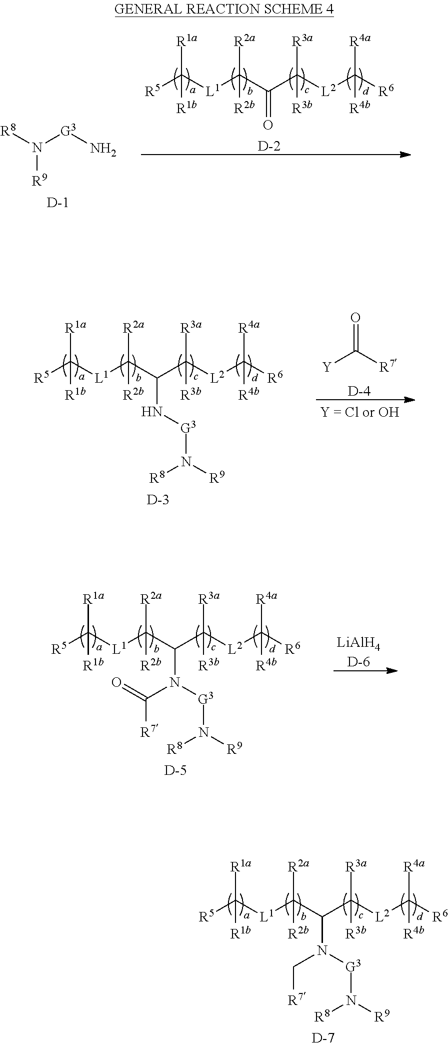

Embodiments of the compound of Formula (II) (e.g., compounds D-5 and D-7) can be prepared according to General Reaction Scheme 4 (“Method D”), wherein R^{1a}, R^{1b}, R^{2a}, R^{2b}, R^{3a}, R^{3b}, R^{4a}, R^{4b}, R⁵, R⁶, R⁸, R⁹, L¹, L², G¹, G², G³, a, b, c and d are as defined herein, and R^{7′} represents R⁷ or a C₃-C₁₉ alkyl. Referring to General Reaction Scheme 1, compounds of structure D-1 and D-2 can be purchased from commercial sources or prepared according to methods familiar to one of ordinary skill in the art. A solution of D-1 and D-2 is treated with a reducing agent (e.g., sodium triacetoxyborohydride) to obtain D-3 after any necessary work up. A solution of D-3 and a base (e.g. trimethylamine, DMAP) is treated with acyl chloride D-4 (or carboxylic acid and DCC) to obtain D-5 after any necessary work up and/or purification. D-5 can be reduced with LiAlH4 D-6 to give D-7 after any necessary work up and/or purification.

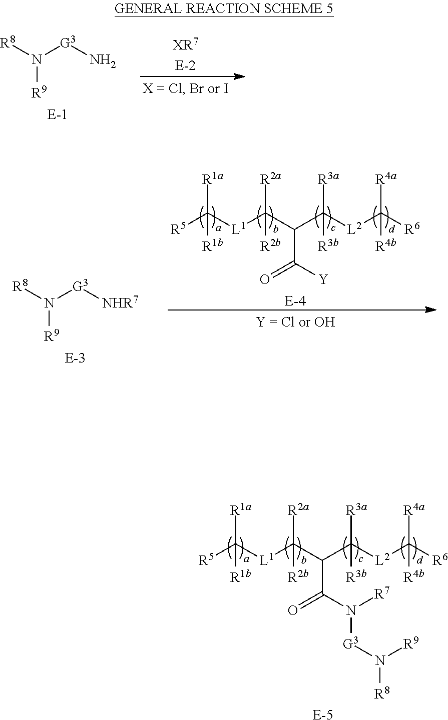

Embodiments of the lipid of Formula (II) (e.g., compound E-5) can be prepared according to General Reaction Scheme 5 (“Method E”), wherein R^{1a}, R^{1b}, R^{2a}, R^{2b}, R^{3a}, R^{3b}, R^{4a}, R^{4b}, R⁵, R⁶, R⁷, R⁸, R⁹, L¹, L², G³, a, b, c and d are as defined herein. Referring to General Reaction Scheme 2, compounds of structure E-1 and E-2 can be purchased from commercial sources or prepared according to methods familiar to one of ordinary skill in the art. A mixture of E-1 (in excess), E-2 and a base (e.g., potassium carbonate) is heated to obtain E-3 after any necessary work up. A solution of E-3 and a base (e.g. trimethylamine, DMAP) is treated with acyl chloride E-4 (or carboxylic acid and DCC) to obtain E-5 after any necessary work up and/or purification.

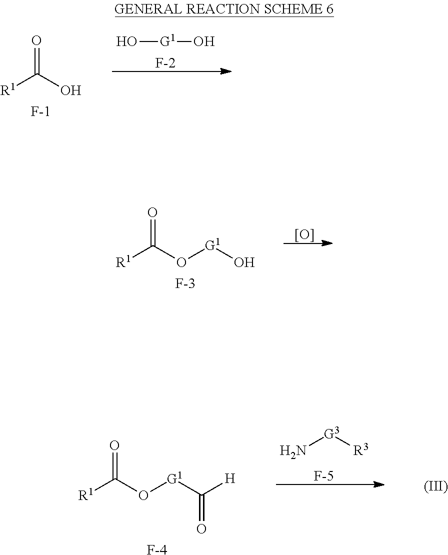

General Reaction Scheme 6 provides an exemplary method (Method F) for preparation of Lipids of Formula (III). G¹, G³, R¹ and R³ in General Reaction Scheme 6 are as defined herein for Formula (III), and G1′ refers to a one-carbon shorter homologue of G1. Compounds of structure F-1 are purchased or prepared according to methods known in the art. Reaction of F-1 with diol F-2 under appropriate condensation conditions (e.g., DCC) yields ester/alcohol F-3, which can then be oxidized (e.g., PCC) to aldehyde F-4. Reaction of F-4 with amine F-5 under reductive amination conditions yields a lipid of Formula (III).

It should be noted that various alternative strategies for preparation of lipids of Formula (III) are available to those of ordinary skill in the art. For example, other lipids of Formula (III) wherein L¹ and L² are other than ester can be prepared according to analogous methods using the appropriate starting material. Further, General Reaction Scheme 6 depicts preparation of a lipids of Formula (III), wherein G¹ and G² are the same; however, this is not a required aspect of the invention and modifications to the above reaction scheme are possible to yield compounds wherein G¹ and G² are different.

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to subjects of all sorts. Modification of pharmaceutical compositions suitable for administration to humans in order to render the compositions suitable for administration to various subjects is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient, which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers. In some embodiments, the formulation may comprise dry particles which comprise the active ingredient and which have a diameter in the range from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. In some embodiments, dry powder compositions include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some instances having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Methods of Treatment or Prevention

The present invention provides methods of inducing an adaptive immune response against influenza virus in a subject comprising administering an effective amount of a composition comprising a combination of at least twenty lipid nanoparticle (LNPs) wherein each LNP comprises a nucleoside-modified RNA encoding at least one influenza virus antigen or a fragment thereof, and further wherein the combination of at least twenty LNPs together comprise at least twenty nucleoside-modified RNA molecule encoding a hemagglutinin (HA) antigen, or fragment thereof for each of influenza A virus H1, H2, H3, H4, H5, H6, H7, H8, H9, H10, H11, H12, H13, H14, H15, H16, H17, and H18, and influenza B virus Vic and Yam.

In one embodiment, the method provides immunity in the subject to multiple strains of influenza virus, influenza virus infection, or to a disease or disorder associated with influenza virus. The present invention thus provides a method of treating or preventing the infection, disease, or disorder associated with influenza virus.

In one embodiment, the composition is administered to a subject having an infection, disease, or disorder associated with influenza virus. In one embodiment, the composition is administered to a subject at risk for developing the infection, disease, or disorder associated with influenza virus. For example, the composition may be administered to a subject who is at risk for being in contact with influenza virus. In one embodiment, the composition is administered to a subject who lives in, traveled to, or is expected to travel to a geographic region in which influenza virus is prevalent. In one embodiment, the composition is administered to a subject who is in contact with or expected to be in contact with another person who lives in, traveled to, or is expected to travel to a geographic region in which influenza virus is prevalent. In one embodiment, the composition is administered to a subject who has knowingly been exposed to influenza virus through their occupation, or other contact.

In one embodiment, the method comprises administering a composition comprising one or more nucleoside-modified nucleic acid molecules encoding one or more influenza virus antigens. In one embodiment, the method comprises administering a composition comprising a first nucleoside-modified nucleic acid molecule encoding one or more influenza virus antigens and a second nucleoside-modified nucleic acid molecule encoding one or more influenza virus antigens. In one embodiment, the method comprises administering a composition comprising a one or more nucleoside-modified nucleic acid molecules encoding a plurality of lineage influenza virus antigens described herein.

In one embodiment, the method comprises administering one or more compositions, each composition comprising one or more nucleoside-modified nucleic acid molecules encoding one or more influenza virus antigens. In one embodiment, the method comprises administering a first composition comprising one or more nucleoside-modified nucleic acid molecules encoding one or more influenza virus antigens and administering a second composition comprising one or more nucleoside-modified nucleic acid molecules encoding one or more influenza virus antigens. In one embodiment, the method comprises administering a plurality of compositions, each composition comprising one or more nucleoside-modified nucleic acid molecules encoding one or more lineage influenza virus antigens described herein. In some embodiments, the method comprises a staggered administration of the plurality of compositions.

In some embodiments, the method comprises administering to subject a plurality of nucleoside-modified nucleic acid molecules encoding a plurality of influenza virus antigens, adjuvants, or a combination thereof.

In some embodiments, the method of the invention allows for sustained expression of the influenza virus antigen or adjuvant, described herein, for at least several days following administration. In some embodiments, the method of the invention allows for sustained expression of the influenza virus antigen or adjuvant, described herein, for at least 2 weeks following administration. In some embodiments, the method of the invention allows for sustained expression of the influenza virus antigen or adjuvant, described herein, for at least 1 month following administration. However, the method, in some embodiments, also provides for transient expression, as in some embodiments, the nucleic acid is not integrated into the subject genome.

In some embodiments, the method comprises administering nucleoside-modified RNA, which provides stable expression of the influenza virus antigen or adjuvant described herein. In some embodiments, administration of nucleoside-modified RNA results in little to no innate immune response, while inducing an effective adaptive immune response.

In some embodiments, the method provides sustained protection against influenza virus. For example, in some embodiments, the method provides sustained protection against influenza virus for more than 2 weeks. In some embodiments, the method provides sustained protection against influenza virus for 1 month or more. In some embodiments, the method provides sustained protection against influenza virus for 2 months or more. In some embodiments, the method provides sustained protection against influenza virus for 3 months or more. In some embodiments, the method provides sustained protection against influenza virus for 4 months or more. In some embodiments, the method provides sustained protection against influenza virus for 5 months or more. In some embodiments, the method provides sustained protection against influenza virus for 6 months or more. In some embodiments, the method provides sustained protection against influenza virus for 1 year or more.

In one embodiment, a single immunization of the composition induces a sustained protection against influenza virus for 1 month or more, 2 months or more, 3 months or more, 4 months or more, 5 months or more, 6 months or more, or 1 year or more.

Administration of the compositions of the invention in a method of treatment can be achieved in a number of different ways, using methods known in the art. In one embodiment, the method of the invention comprises systemic administration of the subject, including for example enteral or parenteral administration. In some embodiments, the method comprises intradermal delivery of the composition. In another embodiment, the method comprises intravenous delivery of the composition. In some embodiments, the method comprises intramuscular delivery of the composition. In one embodiment, the method comprises subcutaneous delivery of the composition. In one embodiment, the method comprises inhalation of the composition. In one embodiment, the method comprises intranasal delivery of the composition.

It will be appreciated that the composition of the invention may be administered to a subject either alone, or in conjunction with another agent.

The therapeutic and prophylactic methods of the invention thus encompass the use of pharmaceutical compositions encoding an influenza virus antigen, adjuvant, or a combination thereof, described herein to practice the methods of the invention. The pharmaceutical compositions useful for practicing the invention may be administered to deliver a dose of from 1 ng/kg/day and 100 mg/kg/day. In one embodiment, the invention envisions administration of a dose, which results in a concentration of the compound of the present invention from 10 nM and 10 μM in a mammal.

Typically, dosages which may be administered in a method of the invention to a mammal, such as a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. In some embodiments, the dosage of the compound will vary from about 0.1 μg to about 10 mg per kilogram of body weight of the mammal. In some embodiments, the dosage will vary from about 1 μg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months, several years, or even less frequently, such as every 10-20 years, 15-30 years, or even less frequently, such as every 50-100 years. The frequency of the dose will be readily apparent to the skilled artisan and will depend upon any number of factors, such as, but not limited to, the type and severity of the disease being treated, the type and age of the mammal, etc.

In some embodiments, administration of an immunogenic composition or vaccine of the present invention may be performed by single administration or boosted by multiple administrations.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Development of a Multivalent Nucleoside-Modified mRNA Vaccine to Protect Against all Known Influenza Virus Subtypes

In this report, an alternative strategy is presented for inducing ‘universal’ immunity against distinct influenza virus strains. Previously it has been demonstrated that mRNA-LNP vaccines expressing HA and conserved influenza virus antigens are immunogenic in mice (Pardi et al., 2018, Nat Commun, 9:3361; Freyn et al., 2020, Mol Ther, 28:1569-1584). Instead of focusing on immunogens to elicit antibodies against epitopes that are conserved among many different influenza virus strains, a new vaccine has been designed that encodes separate immunogens from all know IAV subtypes and IBV lineages. Previous studies have shown that cocktails of virus-like particles encoding antigens from 4 different influenza virus subtypes are immunogenic in mice when delivered intranasally (Schwartzman et al., 2015, mBio, 6:e01044). The studies presented herein indicate that antigens from at least 20 distinct influenza viruses can be simultaneously delivered via mRNA-LNPs. The production and standardization of different antigens expressed by mRNA-LNP vaccines is simpler compared to other vaccine approaches (Pardi et al., 2020, Curr Opin Immunol, 65:14-20; Pardi et al., 2018, Nat Rev Drug Discov, 17:261-279), and there may be unique properties of mRNA vaccines that allow the induction of immune responses to multiple antigens without noticeable immunodominance biases. For example, it has previously been reported that mRNA-LNP vaccines induce long-lived germinal center reactions in mice (Pardi et al., 2018, J Exp Med, 215:1571-1588), a finding that has been recently found to occur in SARSCoV2 mRNA vaccinated humans as well (Turner et al., 2021, Nature, 595:421-425).

Mice receiving passively transferred 20 HA mRNA-LNP elicited antibodies survived longer relative to naïve mice following challenge with highly lethal mismatched A/Puerto Rico/8/1934 strain, but most of these mice eventually succumbed to infection. These data suggest that both antibodies and cellular immunity may be required for full protection afforded by the 20 HA mRNA vaccine. Antigenically mismatched challenge strains were used in these experiments to mimic a new pandemic involving a previously unknown viral strain. It is likely that mRNA influenza vaccines that are mismatched to infecting strains do not completely prevent infections, but instead, limit disease severity and protect against death through combined humoral and cellular immune responses. Consistent with this, similar levels of virus were found in nasal washes of vaccinated and non-vaccinated ferrets at early time points following challenge with an antigenically mismatched A/Ruddy turnstone/Delaware/300/2009 avian H1N1 strain. However, vaccinated ferrets cleared virus faster, displayed fewer and less severe symptoms, and survived the H1N1 infection. This may be similar to the ongoing situation with SARS-CoV-2 delta variant infections in humans previously immunized with SARS-CoV-2 mRNA vaccines. In most cases, symptoms and severity are greatly reduced and virus is cleared faster in vaccinated individuals infected with the antigenically drifted SARS-CoV-2 delta variant (Sheikh et al., 2021, Lancet, 397:2461-2462; Lopez Bernal et al., 2021, N Engl J Med, 385:585-594). The overall approach will likely be useful for infectious diseases other than influenza viruses. Multivalent mRNA-LNP vaccines may be applied against other variable pathogens, such as coronaviruses and rhinoviruses. Additional studies will be required to determine the maximum number of antigens that can be simultaneously delivered via mRNA-LNP vaccines and the underlying immunological mechanisms that allow the induction of responses against multiple antigens.

The materials and methods used for the experiments are now described

### Murine Experiments

Female 6-8 week old C57BL/6 mice were purchased from Charles River Laboratories. mRNALNPs were diluted in PBS and injected into animals intramuscularly (i.m.) into the hind leg. For the 20 HA mRNA-LNP vaccinations, individual HA mRNA-LNPs were pooled before vaccination. After vaccination, blood samples were obtained at different days from the submandibular vein and serum was isolated for antigenic analyses. For some experiments, mice were then anesthetized with isoflurane and intranasally inoculated with 500 (TCID)₅₀ of A/Puerto Rico/8/1934 H1N1 influenza virus in 50 μL PBS. Clinical severity scores were calculated by monitoring mice for lethargy, hunched posture, ruffled fur, and labored breathing. Weight loss and survival were also monitored. For some experiments, 800 μl serum from mice immunized with the 20 HA mRNA-LNP vaccine was passively transferred into naïve mice 5 hours prior to A/Puerto Rico/8/1934 infection. All murine experiments were approved by the Institutional Animal Care and Use Committees of the Wistar Institute and the University of Pennsylvania.

### Ferret Experiments

Six month old male ferrets were purchased from Triple F Farms (Sayre, PA, USA). All ferrets were screened for antibodies against circulating influenza A and B viruses, as determined by hemagglutinin inhibition assays using the following antigens obtained through the International Reagent Resource, Influenza Division, WHO Collaborating Center for Surveillance, Epidemiology and Control of Influenza, Centers for Disease Control and Prevention, Atlanta, GA, USA: 2018-2019 WHO Antigen, Influenza A (H3) Control Antigen (A/Singapore/INFIMH-16-0019/2016), BPL-Inactivated, FR-1606; 2014-2015 WHO Antigen, Influenza A (H1N1)pdm09 Control Antigen (A/California/07/2009 NYMC X-179A), BPL-Inactivated, FR-1184; 2018-2019 WHO Antigen, Influenza B Control Antigen, Victoria Lineage (B/Colorado/06/2017), BPL-Inactivated, FR-1607; 2015-2016 WHO Antigen, Influenza B Control Antigen, Yamagata Lineage (B/Phuket/3073/2013), BPL-Inactivated, FR-1403. Vaccinations were administered i.m. and infections were delivered i.n. in a total volume of 500 μL. Clinical symptoms such as weight loss, temperature, Reuman activity score and other symptoms such as sneezing, coughing, lethargy or nasal discharge were recorded during each procedure. Animals were given A/D diet twice a day to entice eating once they reached 10% weight loss and Ringer's lactated solution was administered subcutaneously up to twice daily upon observed dehydration. Humane endpoints for this study included body weight loss exceeding 20% (relative to weight at challenge) and a prolonged clinical activity score of 3. Animals were sedated using isoflurane for all nasal washes and survival blood draws. Ketamine and xylazine were used for sedation for all terminal procedures followed by cardiac administration of euthanasia solution.

### Recombinant HA Proteins

Recombinant full-length HA proteins and ‘headless’ HA proteins were used as antigens in ELISAs and absorption assays. For full-length HA proteins, plasmids were created with codon optimized full length HA sequences and HA transmembrane domains were replaced with a FoldOn trimerization domain from T4 fibritin, an AviTag site specific biotinylation sequence, and a hexahistidine tag, as previously described (Whittle et al., 2014, J Virol, 88:4047-4057). Plasmids were obtained encoding recombinant headless H1 and H3 HA proteins from Adrian McDermott and Barney Graham from the Vaccine Research Center at the National Institutes of Health (5, 6). Plasmids were transfected into 293F suspension cells and supernatants were isolated four days later and clarified by centrifugation. HA proteins were purified from supernatants by Ni-NTA affinity chromatography (Qiagen). For some experiments, proteins were biotinylated using the Avidity BirA-500 kit.

### Standard ELISAs

96 well ELISA plates (Immulon) were coated with 50 μL of recombinant proteins in PBS at 2 μg/mL and incubated overnight at 4° C. The day of the experiment, plates were blocked with 150 μL of PBS-0.01% Tween 20, 3% Normal Goat serum, and 0.5% Milk powder, and incubated for 1 hour at room temperature. Two-fold serial dilutions of samples in blocking buffer were added to the plates and allowed to incubate for 2 hours at room temperature. Plates were then incubated with peroxidase-conjugated goat anti-mouse IgG (Jackson), or peroxidase-conjugated goat antiferret IgG (Abcam). SureBlue TMB peroxidase substrate (KPL) was added to each well, and the reaction was then stopped with the addition of a 250 mM HCl solution. Absorbance was read at 450 nm using a plate reader (Molecular Devices). Plates were washed three times with PBS-0.01% Tween 20 between each step. Background signals at each dilution were subtracted for each sample and area under the curve (AUC) was calculated using GraphPad Prism. All background subtracted data below y=0 were excluded and data were represented as mean±SEM.

### Biotinylated ELISAs

For some ELISAs, streptavidin plates coated with biotinylated HA proteins were used. For this, flat bottom 96 well ELISA plates (Immulon) were coated with 50 μL of 2 μg/mL of streptavidin diluted in PBS, and plates were incubated with streptavidin at 37° C. overnight. The day of the experiment, plates were washed and then incubated for 1 hour at room temperature with 50 μL of 2 μg/mL biotinylated protein in TBS-0.01% Tween20 and 0.1% BSA. Plates were then blocked with TBS-0.01% Tween20 and 1% BSA for 1 hour at room temperature. Two-fold serial dilutions of samples were added to the plates and incubated for 2 hours at room temperature. Plates were then incubated with peroxidase-conjugated goat anti-mouse IgG (Jackson), or peroxidaseconjugated goat anti-ferret IgG (Abcam). SureBlue TMB peroxidase substrate (KPL) was added to each well, and the reaction was then stopped with the addition of 250 mM HCl solution. Absorbance was read at 450 nm using a plate reader (Molecular Devices). Plates were washed three times with PBS-0.01% Tween 20 between each step. Data were analyzed using Prism 8.0 (GraphPad), and the area under the curve (AUC) was calculated. All background subtracted data below y=0 were excluded and data were represented as mean±SEM.

### HAI Assays

Sera samples were pretreated with receptor-destroying enzyme (Denka Seiken) followed by hemadsorption. HAI titrations were performed in 96-well round plates (Corning). Sera were serially diluted twofold and added to four agglutinating doses of virus in a total volume of 100 μL. Next, 12.5 μL of a 2% (vol/vol) turkey erythrocyte solution was added. The sera, virus, and erythrocytes were mixed, and the assay was read out after incubating for 60 minutes at room temperature. HAI titers were recorded as the inverse of the highest antibody dilution that inhibited four agglutinating doses of virus. HAI experiments were completed with viruses with HA from A/Michigan/45/2015, A/Singapore/INFIMH-16-0019/2016, or A/Puerto Rico/8/1934.

### Absorption Assays

Streptavidin M-280 Dynabeads (ThermoFisher) were couple to biotinylated recombinant HA proteins according to manufacturer's instructions. Briefly, beads were washed with PBS+0.1% BSA. Excess biotinylated recombinant HA proteins from A/Michigan/45/2015 (H1) or A/Singapore/INFIMH-16-0019/2016 (H3) were coupled to beads at a concentration of 0.2 μg/μL. Proteins were allowed to incubate with beads while rotating for 30 minutes at room temperature. Beads were separated from unbound proteins using a magnet, washed, and resuspended in PBS+0.1% BSA. Serum samples were diluted 1:25 in PBS and incubated with protein coupled beads at a ratio of 1:2. The bead and sample mixtures were incubated for 1 hour at room temperature while shaking at 800 rpm. After this incubation, antibodies bound to beads were removed using a magnet. The remaining unbound antibody fractions were used in serological assays.

The results of the experiments are now described

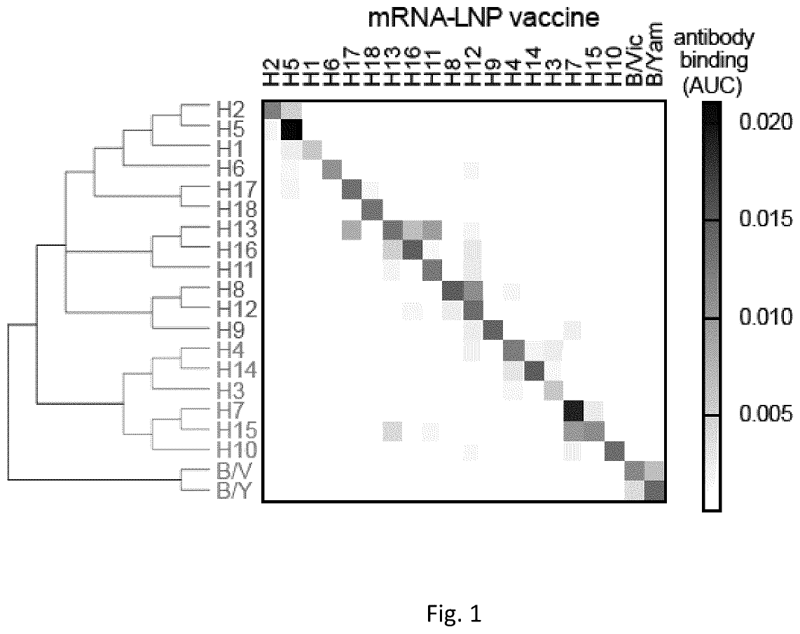

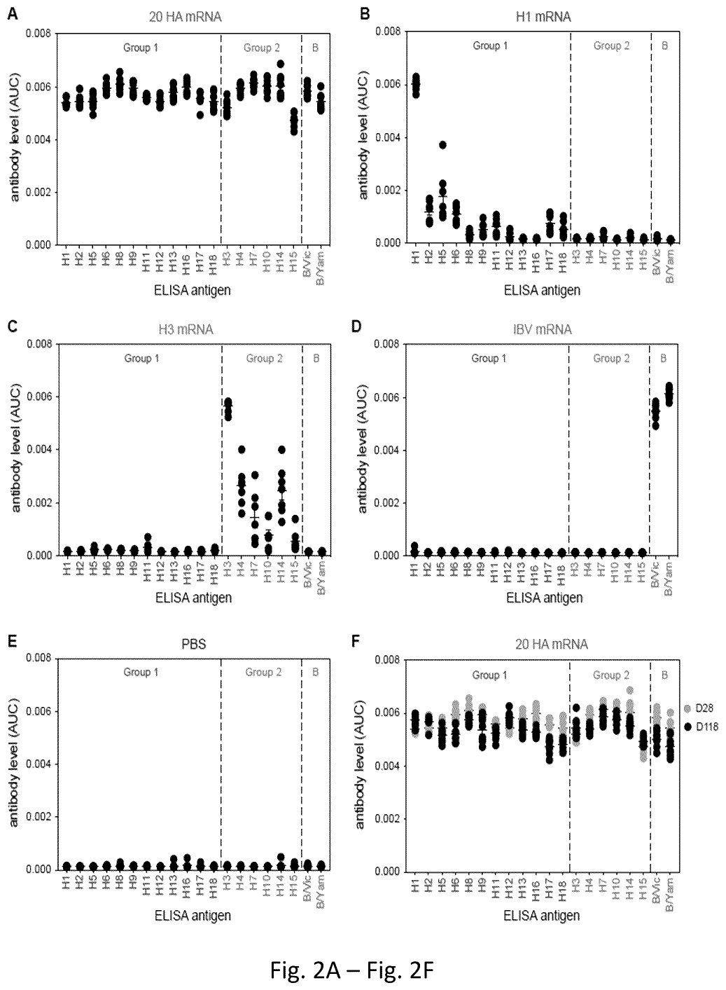

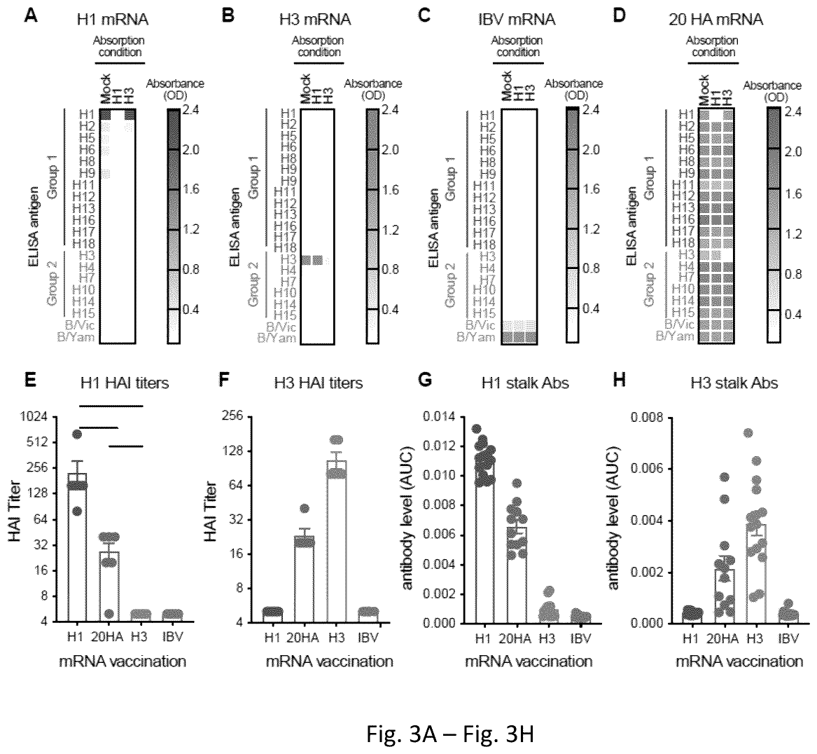

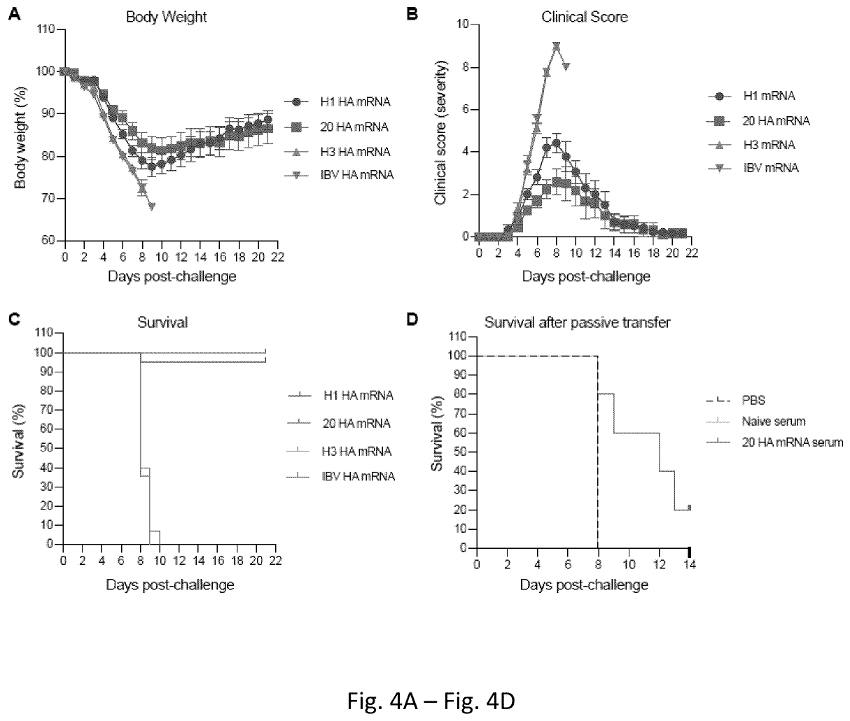

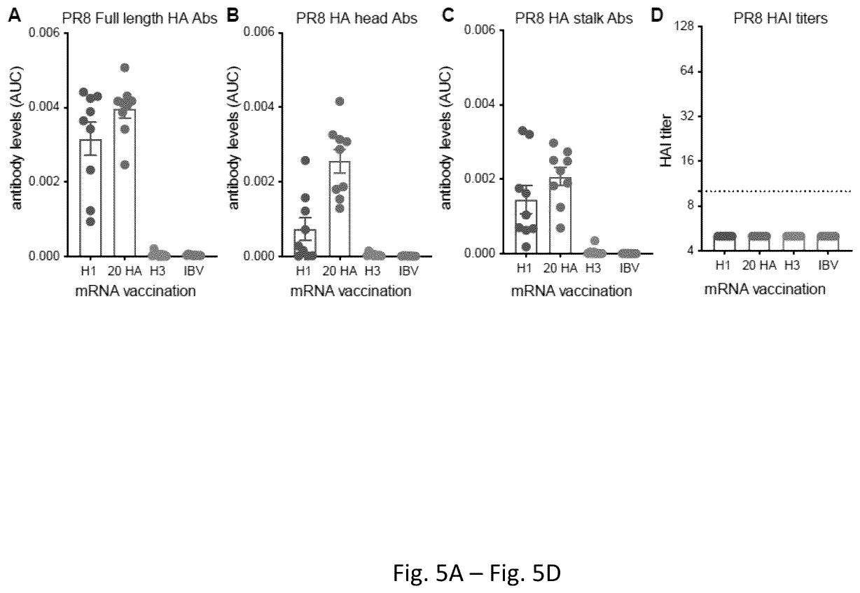

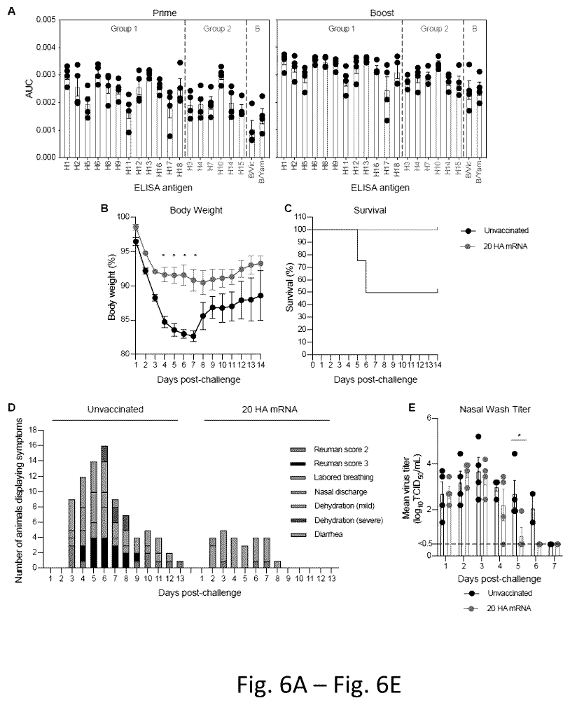

A nucleoside-modified mRNA-LNP vaccine was developed expressing HA antigens from all known influenza virus subtypes to determine if antibodies against multiple mRNA-expressed antigens can be elicited simultaneously. 20 different HA nucleoside-modified mRNAs were prepared using a T7 RNA polymerase on linearized plasmids and each individual mRNA was encapsulated in LNPs as previously described (8). A representative HA from each IAV subtype and IBV lineage was included (FIG. 1). Groups of mice were vaccinated intramuscularly with a low dose (3 μg) of each individual HA mRNA vaccine to verify that each mRNA vaccine component was immunogenic. Antibodies reactive to all 20 HA vaccine components in serum obtained 28 days after vaccination were quantified (FIG. 1). Each individual HA mRNA vaccine elicited antibodies that reacted more efficiently to the encoded HA compared to other HAs that were tested. A low level of cross-reactivity was found among antibodies elicited by single HA mRNA vaccinations, which is consistent with previous studies (8) demonstrating that higher doses of vaccines are required to elicit antibodies that target the HA stalk. Mice were then vaccinated with all 20 HA mRNA-LNPs simultaneously. For these experiments, mice were vaccinated intramuscularly with a combined dose of 50 μg of HA mRNA (2.5 μg of each individual HA mRNA-LNP) and the levels of serum antibodies were quantified 28 days after vaccination (FIG. 2A). As a control, mice were vaccinated with a 50 μg dose of mRNA-LNPs encoding single HAs from H1N1 (A/Michigan/45/2015) (FIG. 2B), H3N2 (A/Singapore/INFIMH/16/2016) (FIG. 2C), or IBV (B/Phuket/3073/2013) (FIG. 2D). Mice vaccinated with the 20 HA mRNA-LNPs produced antibodies that reacted to all 20 encoded HAs (FIG. 2A). The broad reactivity of antibodies induced by the 20 HA mRNA-LNP vaccine was not simply due to the higher 50 μg dose of mRNA-LNP vaccine used in these experiments. Mice that were vaccinated with a 50 μg dose of H1 mRNA-LNP produced antibodies that reacted strongly to H1 with lower reactivity to other group 1 HAs and minimal reactivity to group 2 HAs and IBV HAs (FIG. 2B). Similarly, mice vaccinated with a 50 μg dose of H3 mRNA-LNP produced antibodies that reacted strongly to H3 with lower reactivity to other group 2 HAs and minimal reactivity to group 1 HAs and IBV HAs (FIG. 2C). Mice vaccinated with a 50 μg dose of IBV mRNA-LNP produced antibodies that reacted strongly to IBV HAs (FIG. 2D) and mice that were vaccinated with PBS did not produce detectable influenza virus-reactive antibodies (FIG. 2E). Antibody levels in mice immunized with the 20 HA mRNA-LNP vaccine remained largely unchanged 4 months post-vaccination (FIG. 2F). Absorption assays were completed to determine the level of cross-subtype reactivity of antibodies elicited by vaccination. For these experiments, serum samples were absorbed with magnetic beads that were coupled with different recombinant HAs. ELISAs were then completed with serum antibodies that remained after absorption. As expected, H1-coupled beads were able to deplete H1-reactive serum antibodies from mice that received an H1 mRNA-LNP vaccine (FIG. 3A) and H3-coupled beads were able to deplete H3-reactive serum antibodies from mice that received an H3 mRNA-LNP vaccine (FIG. 3B). Neither H1- or H3-coupled beads depleted IBV HA-reactive antibodies elicited by an IBV mRNA-LNP vaccine (FIG. 3C). H1-coupled beads efficiently depleted H1-reactive antibodies and H3-couple beads efficiently depleted H3-reactive antibodies in the serum of mice vaccinated with the 20 HA mRNA-LNP vaccine, but these absorptions did not substantially decrease binding of antibodies reactive to other HAs in the testing panel (FIG. 3D). These data indicate that the 20 HA mRNA-LNP vaccine elicits antibodies reactive to distinct HAs, rather than purely cross-reactive antibodies that react to all HA subtypes. Additional assays were completed to further characterize the specificity of H1 and H3-reactive antibodies elicited by vaccination. Using serum samples collected 28 days post-vaccination, hemagglutination inhibition (HAI) assays were completed to detect antibodies targeting the HA globular head (10) and additional ELISA analyses with “headless HAs” (5, 6) to detect HA stalk-reactive antibodies. The 20 HA mRNA-LNP vaccine elicited H1 and H3 head and stalk-reactive antibodies (FIG. 3E-FIG. 3H). As expected H1 and H3 head and stalk-reactive serum antibodies were at lower levels in mice receiving the 20 HA mRNA-LNP vaccine (which contained only 2.5 μg of H1 mRNA and 2.5 μg of H3 mRNA) compared to mice receiving 50 μg of H1 or H3 mRNA LNPs (FIG. 3E-FIG. 3F). Taken together, these data indicate that mRNA vaccines can successfully deliver at least 20 distinct antigens without noticeable immunodominance biases or immune focusing on epitopes conserved among the different antigens. Mice ˜4 months were challenged after vaccination with the highly mouse-adapted A/Puerto Rico/8/1934 H1N1 influenza A virus strain. The HA of A/Puerto Rico/8/1934 H1N1 virus is antigenically distinct compare to the A/Michigan/45/2015 H1 component of the 20 HA mRNA-LNP vaccine, and therefore this challenge experiment mimics a pandemic situation where the viral strain is imperfectly matched to the vaccine antigens. Mice vaccinated with H3 mRNA-LNP or IBV mRNA-LNP rapidly lost weight, displayed clinical symptoms, and died between 8-10 days after infection (FIG. 4A-FIG. 4C). Conversely, mice vaccinated with H1 mRNA-LNP or the 20 HA mRNA-LNP initially lost weight (FIG. 4A) and were symptomatic (FIG. 4B) following vaccination but then began recovering 8-10 days after infection. Interestingly, mice receiving the 20 HA mRNA-LNP vaccine were less symptomatic compared to mice receiving H1 mRNA-LNP vaccines between 5-8 days after infection (FIG. 4B), although the immunological basis of this is unknown. All of the mice vaccinated with the 20 HA mRNA-LNP vaccine and 95% of mice vaccinated with the H1 mRNA-LNP vaccine survived after A/Puerto Rico/8/1934 H1N1 challenge (FIG. 4C). Animals vaccinated with the 20 HA mRNA-LNP and H1 mRNALNP produced antibodies that bound to epitopes in the HA head and stalk (FIG. 5A-FIG. 5C) but these antibodies could not inhibit agglutination of red blood cells (FIG. 5D). It is likely that both antibody and cellular responses elicited by the 20 HA mRNA-LNP vaccine contributed to protection since passive transfer of serum from vaccinated mice into naïve mice only partially protected against A/Puerto Rico/8/1934 infection (FIG. 4D). Finally, a prime/boost vaccination experiment was completed in ferrets to mimic the dosing schedule currently employed for SARS-CoV-2 mRNA vaccines (Freyn et al., 2020, Mol Ther, 28:1569-1584; Topol, 2021, Cell, 184:1401). Animals were vaccinated intramuscularly with 60 μg of the 20 HA mRNA-LNP vaccine and boosted intramuscularly with the same dose 28 days later. Each ferret produced antibodies reactive to all 20 HAs after a single vaccination, and antibody levels increased after the booster vaccination (FIG. 6A). Similar to the murine experiments, ferrets were challenged with an H1N1 virus that was distinct from the A/Michigan/45/2015 H1 that was included in the vaccine. Ferrets were infected with the A/Ruddy turnstone/Delaware/300/2009 avian H1N1 virus and virus was quantified in nasal washes, monitored weight loss, survival, and clinical scores. Unvaccinated animals rapidly lost weight and 2 out of 4 animals died after infection (FIG. 6B-FIG. 6C). The 2 unvaccinated ferrets who did not succumb to infection were given fluids intravenously and offered soft food. Vaccinated ferrets lost less weight and all animals survived following infection without intervention (FIG. 6B-FIG. 6C). Unvaccinated animals displayed more symptoms relative to vaccinated animals after infection (FIG. 6D). Viral titers in nasal washes were similar in unvaccinated and vaccinated animals at days 1-4 after infection, but virus was cleared more efficiently in vaccinated animals at days 5 and 6 after infection (FIG. 6E).

### Example 2: Sequences

2904_pUC-ccTEV-A-bat-Peru-33-10 HA-A101
(SEQ ID NO: 1)
ATGGCCATCTCCGGCGTGCCCGTGCTGGGCTTCTTCATCATCGCCGTGCTGATGTCCGCCCAGGAGTCCTG
GGCCGACCAGATCTGCATCGGCTACCACTCCAACAACTCCACCCAGACCGTGAACACCCTGCTGGAGTCCA
ACGTGCCCGTGACCTCCTCCCACTCCATCCTGGAGAAGGAGCACAACGGCCTGCTGTGCAAGCTGAAGGGC
AAGGCCCCCCTGGACCTGATCGACTGCTCCCTGCCCGCCTGGCTGATGGGCAACCCCAAGTGCGACGAGCT
GCTGACCGCCTCCGAGTGGGCCTACATCAAGGAGGACCCCGAGCCCGAGAACGGCATCTGCTTCCCCGGCG
ACTTCGACTCCCTGGAGGACCTGATCCTGCTGGTGTCCAACACCGACCACTTCCGCAAGGAGAAGATCATC
GACATGACCCGCTTCTCCGACGTGACCACCAACAACGTGGACTCCGCCTGCCCCTACGACACCAACGGCGC
CTCCTTCTACCGCAACCTGAACTGGGTGCAGCAGAACAAGGGCAAGCAGCTGATCTTCCACTACCAGAACT
CCGAGAACAACCCCCTGCTGATCATCTGGGGCGTGCACCAGACCTCCAACGCCGCCGAGCAGAACACCTAC
TACGGCTCCCAGACCGGCTCCACCACCATCACCATCGGCGAGGAGACCAACACCTACCCCCTGGTGATCTC
CGAGTCCTCCATCCTGAACGGCCACTCCGACCGCATCAACTACTTCTGGGGCGTGGTGAACCCCAACCAGA
ACTTCTCCATCGTGTCCACCGGCAACTTCATCTGGCCCGAGTACGGCTACTTCTTCCAGAAGACCACCAAC
ATCTCCGGCATCATCAAGTCCTCCGAGAAGATCTCCGACTGCGACACCATCTGCCAGACCAAGATCGGCGC
CATCAACTCCACCCTGCCCTTCCAGAACATCCACCAGAACGCCATCGGCGACTGCCCCAAGTACGTGAAGG
CCCAGGAGCTGGTGCTGGCCACCGGCCTGCGCAACAACCCCATCAAGGAGACCCGCGGCCTGTTCGGCGCC
ATCGCCGGCTTCATCGAGGGCGGCTGGCAGGGCCTGATCGACGGCTGGTACGGCTACCACCACCAGAACTC
CGAGGGCTCCGGCTACGCCGCCGACAAGGAGGCCACCCAGAAGGCCGTGGACGCCATCACCACCAAGGTGA
ACAACATCATCGACAAGATGAACACCCAGTTCGAGTCCACCGCCAAGGAGTTCAACAAGATCGAGATGCGC
ATCAAGCACCTGTCCGACCGCGTGGACGACGGCTTCCTGGACGTGTGGTCCTACAACGCCGAGCTGCTGGT
GCTGCTGGAGAACGAGCGCACCCTGGACTTCCACGACGCCAACGTGAACAACCTGTACCAGAAGGTGAAGG
TGCAGCTGAAGGACAACGCCATCGACATGGGCAACGGCTGCTTCAAGATCCTGCACAAGTGCAACAACACC
TGCATGGACGACATCAAGAACGGCACCTACAACTACTACGAGTACCGCAAGGAGTCCCACCTGGAGAAGCA
GAAGATCGACGGCGTGAAGCTGTCCGAGAACTCCTCCTACAAGATCATGATCATCTACTCCACCGTGGCCT
CCTCCGTGGTGCTGGGCCTGATCATCCTGGCCGCCATCGAGTGGGGCTGCTTCAAGGGCAACCTGCAGTGC
CGCATCTGCATCtaa
2905_pUC-ccTEV-A-black headed gull-Sweden-5-99 HA-A101
(SEQ ID NO: 2)
ATGGCCATCTCCGGCGTGCCCGTGCTGGGCTTCTTCATCATCGCCGTGCTGATGTCCGCCCAGGAGTCCTG
GGCCGACAAGATCTGCATCGGCTACCTGTCCAACAACGCCACCGACACCGTGGACACCCTGACCGAGAACG
GCGTGCCCGTGACCTCCTCCGTGGACCTGGTGGAGACCAACCACACCGGCACCTACTGCTCCCTGAACGGC
ATCTCCCCCATCCACCTGGGCGACTGCTCCTTCGAGGGCTGGATCGTGGGCAACCCCTCCTGCGCCACCAA
CATCAACATCCGCGAGTGGTCCTACCTGATCGAGGACCCCAACGCCCCCAACAAGCTGTGCTTCCCCGGCG
AGCTGGACAACAACGGCGAGCTGCGCCACCTGTTCTCCGGCGTGAACTCCTTCTCCCGCACCGAGCTGATC
TCCCCCAACAAGTGGGGCGACATCCTGGACGGCGTGACCGCCTCCTGCCGCGACAACGGCGCCTCCTCCTT
CTACCGCAACCTGGTGTGGATCGTGAAGAACAAGAACGGCAAGTACCCCGTGATCAAGGGCGACTACAACA
ACACCACCGGCCGCGACGTGCTGGTGCTGTGGGGCATCCACCACCCCGACACCGAGACCACCGCCATCAAC
CTGTACGCCTCCAAGAACCCCTACACCCTGGTGTCCACCAAGGAGTGGTCCAAGCGgTACGAGCTGGAGAT
CGGCACCCGCATCGGCGACGGCCAGCGgTCCTGGATGAAGCTGTACTGGCACCTGATGCGCCCCGGCGAGC
GCATCATGTTCGAGTCCAACGGCGGCCTGATCGCCCCCCGCTACGGCTACATCATCGAGAAGTACGGCACC
GGCCGCATCTTCCAGTCCGGCGTGCGCATGGCCAAGTGCAACACCAAGTGCCAGACCTCCCTGGGCGGCAT
CAACACCAACAAGACCTTCCAGAACATCGAGCGCAACGCCCTGGGCGACTGCCCCAAGTACATCAAGTCCG
GCCAGCTGAAGCTGGCCACCGGCCTGCGCAACGTGCCCTCCGTGGGCGAGCGCGGCCTGTTCGGCGCCATC
GCCGGCTTCATCGAGGGCGGCTGGCCCGGCCTGATCAACGGCTGGTACGGCTTCCAGCACCAGAACGAGCA
GGGCACCGGCATCGCCGCCGACAAGGCCTCCACCCAGAAGGCCATCGACGAGATCACCACCAAGATCAACA
ACATCATCGAGAAGATGAACGGCAACTACGACTCCATCCGCGGCGAGTTCAACCAGGTGGAGAAGCGCATC
AACATGCTGGCCGACCGCGTGGACGACGCCGTGACCGACATCTGGTCCTACAACGCCAAGCTGCTGGTGCT
GCTGGAGAACGGCCGCACCCTGGACCTGCACGACGCCAACGTGCGCAACCTGCACGACCAGGTGAAGCGCA
TCCTGAAGTCCAACGCCATCGACGAGGGCGACGGCTGCTTCAACCTGCTGCACAAGTGCAACGACTCCTGC
ATGGAGACCATCCGCAACGGCACCTACAACCACGAGGACTACCGCGAGGAGTCCCAGCTGAAGCGCCAGGA
GATCGAGGGCATCAAGCTGAAGTCCGAGGACAACGTGTACAAGGTGCTGTCCATCTACTCCTGCATCGCCT
CCTCCATCGTGCTGGTGGGCCTGATCCTGGCCTTCATCATGTGGGCCTGCTCCAACGGCAACTGCCGCTTC
AACGTGTGCATCtaa
2906_pUC-ccTEV-A-duck-Czech-56 HA-A101
(SEQ ID NO: 3)
ATGGCCATCTCCGGCGTGCCCGTGCTGGGCTTCTTCATCATCGCCGTGCTGATGTCCGCCCAGGAGTCCTG
GGCCCAGAACTACACCGGCAACCCCGTGATCTGCATGGGCCACCACGCCGTGGCCAACGGCACCATGGTGA
AGACCCTGGCCGACGACCAGGTGGAGGTGGTGACCGCCCAGGAGCTGGTGGAGTCCCAGAACCTGCCCGAG
CTGTGCCCCTCCCCCCTGCGCCTGGTGGACGGCCAGACCTGCGACATCATCAACGGCGCCCTGGGCTCCCC
CGGCTGCGACCACCTGAACGGCGCCGAGTGGGACGTGTTCATCGAGCGCCCCAACGCCGTGGACACCTGCT
ACCCCTTCGACGTGCCCGAGTACCAGTCCCTGCGCTCCATCCTGGCCAACAACGGCAAGTTCGAGTTCATC
GCCGAGGAGTTCCAGTGGAACACCGTGAAGCAGAACGGCAAGTCCGGCGCCTGCAAGCGCGCCAACGTGAA
CGACTTCTTCAACCGCCTGAACTGGCTGGTGAAGTCCGACGGCAACGCCTACCCCCTGCAGAACCTGACCA
AGATCAACAACGGCGACTACGCCCGCCTGTACATCTGGGGCGTGCACCACCCCTCCACCGACACCGAGCAG
ACCAACCTGTACAAGAACAACCCCGGCGGCGTGACCGTGTCCACCAAGACCTCCCAGACCTCCGTGGTGCC
CAACATCGGCTCCCGCCCCCTGGTGCGCGGCCAGTCCGGCCGCGTGTCCTTCTACTGGACCATCGTGGAGC
CCGGCGACCTGATCGTGTTCAACACCATCGGCAACCTGATCGCCCCCCGCGGCCACTACAAGCTGAACAAC
CAGAAGAAGTCCACCATCCTGAACACCGCCATCCCCATCGGCTCCTGCGTGTCCAAGTGCCACACCGACAA
GGGCTCCCTGTCCACCACCAAGCCCTTCCAGAACATCTCCCGCATCGCCGTGGGCGACTGCCCCCGCTACG
TGAAGCAGGGCTCCCTGAAGCTGGCCACCGGCATGCGCAACATCCCCGAGAAGGCCTCCCGCGGCCTGTTC
GGCGCCATCGCCGGCTTCATCGAGAACGGCTGGCAGGGCCTGATCGACGGCTGGTACGGCTTCCGCCACCA
GAACGCCGAGGGCACCGGCACCGCCGCCGACCTGAAGTCCACCCAGGCCGCCATCGACCAGATCAACGGCA
AGCTGAACCGCCTGATCGAGAAGACCAACGACAAGTACCACCAGATCGAGAAGGAGTTCGAGCAGGTGGAG
GGCCGCATCCAGGACCTGGAGAAGTACGTGGAGGACACCAAGATCGACCTGTGGTCCTACAACGCCGAGCT
GCTGGTGGCCCTGGAGAACCAGCACACCATCGACGTGACCGACTCCGAGATGAACAAGCTGTTCGAGCGCG
TGCGCCGCCAGCTGCGCGAGAACGCCGAGGACAAGGGCAACGGCTGCTTCGAGATCTTCCACAAGTGCGAC
AACAACTGCATCGAGTCCATCCGCAACGGCACCTACGACCACGACATCTACCGCGACGAGGCCATCAACAA
CCGCTTCCAGATCCAGGGCGTGAAGCTGACCCAGGGCTACATGGACATCATCCTGTGGATCTCCTTCTCCA
TCTCCTGCTTCCTGCTGGTGGCCCTGCTGCTGGCCTTCATCCTGTGGGCCTGCCAGAACGGCAACATCCGC
TGCCAGATCTGCATCtaa
2907_pUC-ccTEV-A-Hong Kong-33982-2009 HA-A101
(SEQ ID NO: 4)
ATGGCCATCTCCGGCGTGCCCGTGCTGGGCTTCTTCATCATCGCCGTGCTGATGTCCGCCCAGGAGTCCTG
GGCCGACAAGATCTGCATCGGCCACCAGTCCACCAACTCCACCGAGACCGTGGACACCCTGACCGAGACCA
ACGTGCCCGTGACCCACGCCAAGGAGCTGCTGCAGACCGAGCACAACGGCATGCTGTGCGCCACCAACCTG
GGCCACCCCCTGATCCTGGACACCTGCACCATCGAGGGCCTGATCTACGGCAACCCCTCCTGCGACCTGCT
GCTGGAGGGCCGCGAGTGGTCCTACATCGTGGAGCGCCCCTCCGCCGTGAACGGCACCTGCTACCCCGGCA
ACGTGGAGAACCTGGAGGAGCTGCGCACCCTGTTCTCCTCCGCCTCCTCCTACCAGCGCATCCAGATCTTC
CCCGACACCACCTGGAACGTGACCTACACCGGCACCTCCCGCTCCTGCTCCGGCTCCTTCTACCGCTCCAT
GCGCTGGCTGACCCAGAAGTCCGGCTTCTACCCCGTGCAGGACGCCAAGTACACCAACAACCGCGGCAAGA
ACATCCTGTTCGTGTGGGGCATCCACCACCCCCACACCTACACCGACCAGACCACCCTGTACATCCGCAAC
GACACCACCACCTCCGTGACCACCGAGGAGCTGAACCGCATCTTCAAGCCCGTGATCGTGCCCCGCCTGCT
GGTGAACGGCCAGCAGGGCCGCATCGACTACTACTGGTCCGTGCTGAAGCCCGGCCAGACCCTGCGCGTGC
GCTCCAACGGCAACCTGATCGCCCCCTGGTACGGCCACGTGCTGTCCGGCGGCTCCCACGGCCGCATCCTG
AAGACCGACCTGAAGTCCGGCAACTGCGTGGTGCAGTGCCAGACCGAGAAGGGCGGCCTGAACTCCACCCT
GCCCTTCCACAACATCTCCAAGTACGCCTTCGGCACCTGCCCCAAGTACGTGAAGGTGAACTCCCTGAAGC
TGGCCATCGGCCTGCGCAACGTGCCCGCCCGCTCCAACCGCGGCCTGTTCGGCGCCATCGCCGGCTTCATC
GAGGGCGGCTGGCCCGGCCTGGTGGCCGGCTGGTACGGCTTCCAGCACTCCAACGACCAGGGCGTGGGCAT
GGCCGCCGACCGCGACTCCACCCAGAAGGCCGTGGACAAGATCACCTCCAAGGTGAACAACATCGTGGACA
AGATGAACAAGCAGTACGAGATCATCGACCACGAGTTCTCCGAGGTGGAGACCCGCCTGAACATGATCAAC
AACAAGATCGACGACCAGATCCAGGACGTGTGGGCCTACAACGCCGAGCTGCTGGTGCTGCTGGAGAACCA
GAAGACCCTGGACGAGCACGACGCCAACGTGAACAACCTGTACAACAAGGTGAAGCGCGCCCTGGGCTCCA
ACGCCATGGAGGACGGCAAGGGCTGCTTCGAGCTGTACCACAAGTGCGACGACCAGTGCATGGAGACCATC
CGCAACGGCACCTACGACCGCCGCAAGTACCGCGAGGAGTCCCGCCTGGAGCGCCAGCGCATCGAGGGCGT
GAAGCTGGAGTCCGAGGGCACCTACAAGATCCTGACCATCTACTCCACCGTGGCCTCCTCCCTGGTGATCG
CCATGGGCTTCGCCGCCTTCCTGTTCTGGGCCATGTCCAACGGCTCCTGCCGCTGCAACATCTGCATCtaa
2908_pUC-ccTEV-A-Japan-305-1957 HA-A101
(SEQ ID NO: 5)
ATGGCCATCTCCGGCGTGCCCGTGCTGGGCTTCTTCATCATCGCCGTGCTGATGTCCGCCCAGGAGTCCTG
GGCCGACCAGATCTGCATCGGCTACCACGCCAACAACTCCACCGAGAAGGTGGACACCATCCTGGAGCGCA
ACGTGACCGTGACCCACGCCAAGGACATCCTGGAGAAGACCCACAACGGCAAGCTGTGCAAGCTGAACGGC
ATCCCCCCCCTGGAGCTGGGCGACTGCTCCATCGCCGGCTGGCTGCTGGGCAACCCCGAGTGCGACCGCCT
GCTGTCCGTGCCCGAGTGGTCCTACATCATGGAGAAGGAGAACCCCCGCGACGGCCTGTGCTACCCCGGCT
CCTTCAACGACTACGAGGAGCTGAAGCACCTGCTGTCCTCCGTGAAGCACTTCGAGAAGGTGAAGATCCTG
CCCAAGGACCGCTGGACCCAGCACACCACCACCGGCGGCTCCCGCGCCTGCGCCGTGTCCGGCAACCCCTC
CTTCTTCCGCAACATGGTGTGGCTGACCAAGAAGGGCTCCAACTACCCCGTGGCCAAGGGCTCCTACAACA
ACACCTCCGGCGAGCAGATGCTGATCATCTGGGGCGTGCACCACCCCAACGACGAGACCGAGCAGCGCACC
CTGTACCAGAACGTGGGCACCTACGTGTCCGTGGGCACCTCCACCCTGAACAAGCGgTCCACCCCCGAGAT
CGCCACCCGCCCCAAGGTGAACGGCCTGGGCGGCCGCATGGAGTTCTCCTGGACCCTGCTGGACATGTGGG
ACACCATCAACTTCGAGTCCACCGGCAACCTGATCGCCCCCGAGTACGGCTTCAAGATCTCCAAGCGCGGC
TCCTCCGGCATCATGAAGACCGAGGGCACCCTGGAGAACTGCGAGACCAAGTGCCAGACCCCCCTGGGCGC
CATCAACACCACCCTGCCCTTCCACAACGTGCACCCCCTGACCATCGGCGAGTGCCCCAAGTACGTGAAGT
CCGAGAAGCTGGTGCTGGCCACCGGCCTGCGCAACGTGCCCCAGATCGAGTCCCGCGGCCTGTTCGGCGCC
ATCGCCGGCTTCATCGAGGGCGGCTGGCAGGGCATGGTGGACGGCTGGTACGGCTACCACCACTCCAACGA
CCAGGGCTCCGGCTACGCCGCCGACAAGGAGTCCACCCAGAAGGCCTTCGACGGCATCACCAACAAGGTGA
ACTCCGTGATCGAGAAGATGAACACCCAGTTCGAGGCCGTGGGCAAGGAGTTCTCCAACCTGGAGCGCCGC
CTGGAGAACCTGAACAAGAAGATGGAGGACGGCTTCCTGGACGTGTGGACCTACAACGCCGAGCTGCTGGT
GCTGATGGAGAACGAGCGCACCCTGGACTTCCACGACTCCAACGTGAAGAACCTGTACGACAAGGTGCGCA
TGCAGCTGCGCGACAACGTGAAGGAGCTGGGCAACGGCTGCTTCGAGTTCTACCACAAGTGCGACGACGAG
TGCATGAACTCCGTGAAGAACGGCACCTACGACTACCCCAAGTACGAGGAGGAGTCCAAGCTGAACCGCAA
CGAGATCAAGGGCGTGAAGCTGTCCTCCATGGGCGTGTACCAGATCCTGGCCATCTACGCCACCGTGGCCG
GCTCCCTGTCCCTGGCCATCATGATGGCCGGCATCTCCTTCTGGATGTGCTCCAACGGCTCCCTGCAGTGC
CGCATCTGCATCtaa
2909_pUC-ccTEV-A-Jiangxi-09037-2014 HA-A101
(SEQ ID NO: 6)
ATGGCCATCTCCGGCGTGCCCGTGCTGGGCTTCTTCATCATCGCCGTGCTGATGTCCGCCCAGGAGTCCTG
GGCCCTGGACAAGATCTGCCTGGGCCACCACGCCGTGGCCAACGGCACCATCGTGAAGACCCTGACCAACG
AGCAGGAGGAGGTGACCAACGCCACCGAGACCGTGGAGTCCACCGGCATCAACCGCCTGTGCATGAAGGGC
CGCAAGCACAAGGACCTGGGCAACTGCCACCCCATCGGCATGCTGATCGGCACCCCCGCCTGCGACCTGCA
CCTGACCGGCACCTGGGACACCCTGATCGAGCGCGAGAACGCCATCGCCTACTGCTACCCCGGCGCCACCG
TGAACGTGGAGGCCCTGCGCCAGAAGATCATGGAGTCCGGCGGCATCGACAAGATCTCCACCGGCTTCACC
TACGGCTCCTCCATCAACTCCGCCGGCACCACCCGCGCCTGCATGCGCAACGGCGGCAACTCCTTCTACGC
CGAGCTGAAGTGGCTGGTGTCCAAGTCCAAGGGCCAGGACTTCCCCCAGACCACCAACACCTACCGCAACA
CCGACACCGCCGAGCACCTGATCATGTGGGGCATCCACCACCCCTCCTCCATCCAGGAGAAGAACGACCTG
TACGGCACCCAGTCCCTGTCCATCTCCGTGGGCTCCTCCACCTACCGCAACAACTTCGTGCCCGTGGTGGG
CGCCCGCCCCCAGGTGAACGGCCAGTCCGGCCGCATCGACTTCCACTGGACCCTGGTGCAGCCCGGCGACA
ACATCACCTTCTCCCACAACGGCGGCCTGATCGCCCCCTCCCGCGTGTCCAAGCTGATCGGCCGCGGCCTG
GGCATCCAGTCCGACGCCCCCATCGACAACAACTGCGAGTCCAAGTGCTTCTGGCGCGGCGGCTCCATCAA
CACCCGCCTGCCCTTCCAGAACCTGTCCCCCCGCACCGTGGGCCAGTGCCCCAAGTACGTGAACCGCCGCT
CCCTGATGCTGGCCACCGGCATGCGCAACGTGCCCGAGCTGATCCAGGGCCGCGGCCTGTTCGGCGCCATC
GCCGGCTTCCTGGAGAACGGCTGGGAGGGCATGGTGGACGGCTGGTACGGCTTCCGCCACCAGAACGCCCA
GGGCACCGGCCAGGCCGCCGACTACAAGTCCACCCAGGCCGCCATCGACCAGATCACCGGCAAGCTGAACC
GCCTGGTGGAGAAGACCAACACCGAGTTCGAGTCCATCGAGTCCGAGTTCTCCGAGATCGAGCACCAGATC
GGCAACGTGATCAACTGGACCATGGACTCCATCACCGACATCTGGACCTACCAGGCCGAGCTGCTGGTGGC
CATGGAGAACCAGCACACCATCGACATGGCCGACTCCGAGATGCTGAACCTGTACGAGCGCGTGCGCAAGC
AGCTGCGCCAGAACGCCGAGGAGGACGGCAAGGGCTGCTTCGAGATCTACCACGCCTGCGACGACTCCTGC
ATGGAGTCCATCCGCAACAACACCTACGACCACTCCCAGTACCGCGAGGAGGCCCTGCTGAACCGCCTGAA
CATCAACCCCGTGACCCTGTCCTCCGGCTACAAGGACATCATCCTGTGGTTCTCCTTCGGCGCCTCCTGCT
TCGTGCTGCTGGCCGTGGTGATGGGCCTGGTGTTCTTCTGCCTGAAGAACGGCAACATGCGCTGCACCATC
TGCATCtaa
2910_pUC-ccTEV-A-mallard-Gurjev-263-82 HA-A101
(SEQ ID NO: 7)
ATGGCCATCTCCGGCGTGCCCGTGCTGGGCTTCTTCATCATCGCCGTGCTGATGTCCGCCCAGGAGTCCTG
GGCCCAGATCACCAACGGCACCACCGGCAACCCCATCATCTGCCTGGGCCACCACGCCGTGGAGAACGGCA
CCTCCGTGAAGACCCTGACCGACAACCACGTGGAGGTGGTGTCCGCCAAGGAGCTGGTGGAGACCAACCAC
ACCGACGAGCTGTGCCCCTCCCCCCTGAAGCTGGTGGACGGCCAGGACTGCCACCTGATCAACGGCGCCCT
GGGCTCCCCCGGCTGCGACCGCCTGCAGGACACCACCTGGGACGTGTTCATCGAGCGCCCCACCGCCGTGG
ACACCTGCTACCCCTTCGACGTGCCCGACTACCAGTCCCTGCGCTCCATCCTGGCCTCCTCCGGCTCCCTG
GAGTTCATCGCCGAGCAGTTCACCTGGAACGGCGTGAAGGTGGACGGCTCCTCCTCCGCCTGCCTGCGCGG
CGGCCGCAACTCCTTCTTCTCCCGCCTGAACTGGCTGACCAAGGCCACCAACGGCAACTACGGCCCCATCA
ACGTGACCAAGGAGAACACCGGCTCCTACGTGCGCCTGTACCTGTGGGGCGTGCACCACCCCTCCTCCGAC
AACGAGCAGACCGACCTGTACAAGGTGGCCACCGGCCGCGTGACCGTGTCCACCCGCTCCGACCAGATCTC
CATCGTGCCCAACATCGGCTCCCGCCCCCGCGTGCGCAACCAGTCCGGCCGCATCTCCATCTACTGGACCC
TGGTGAACCCCGGCGACTCCATCATCTTCAACTCCATCGGCAACCTGATCGCCCCCCGCGGCCACTACAAG
ATCTCCAAGTCCACCAAGTCCACCGTGCTGAAGTCCGACAAGCGCATCGGCTCCTGCACCTCCCCCTGCCT
GACCGACAAGGGCTCCATCCAGTCCGACAAGCCCTTCCAGAACGTGTCCCGCATCGCCATCGGCAACTGCC
CCAAGTACGTGAAGCAGGGCTCCCTGATGCTGGCCACCGGCATGCGCAACATCCCCGGCAAGCAGGCCAAG
GGCCTGTTCGGCGCCATCGCCGGCTTCATCGAGAACGGCTGGCAGGGCCTGATCGACGGCTGGTACGGCTT
CCGCCACCAGAACGCCGAGGGCACCGGCACCGCCGCCGACCTGAAGTCCACCCAGGCCGCCATCGACCAGA
TCAACGGCAAGCTGAACCGCCTGATCGAGAAGACCAACGAGAAGTACCACCAGATCGAGAAGGAGTTCGAG
CAGGTGGAGGGCCGCATCCAGGACCTGGAGAAGTACGTGGAGGACACCAAGATCGACCTGTGGTCCTACAA
CGCCGAGCTGCTGGTGGCCCTGGAGAACCAGCACACCATCGACGTGACCGACTCCGAGATGAACAAGCTGT
TCGAGCGCGTGCGCCGCCAGCTGCGCGAGAACGCCGAGGACCAGGGCAACGGCTGCTTCGAGATCTTCCAC
CAGTGCGACAACAACTGCATCGAGTCCATCCGCAACGGCACCTACGACCACAACATCTACCGCGACGAGGC
CATCAACAACCGCATCAAGATCAACCCCGTGACCCTGACCATGGGCTACAAGGACATCATCCTGTGGATCT
CCTTCTCCATGTCCTGCTTCGTGTTCGTGGCCCTGATCCTGGGCTTCGTGCTGTGGGCCTGCCAGAACGGC
AACATCCGCTGCCAGATCTGCATCtaa
2911_pUC-ccTEV-A-mallard-Interior Alaska-7MP0167-07 HA-A101
(SEQ ID NO: 8)
ATGGCCATCTCCGGCGTGCCCGTGCTGGGCTTCTTCATCATCGCCGTGCTGATGTCCGCCCAGGAGTCCTG
GGCCTACGACAAGATCTGCATCGGCTACCAGACCAACAACTCCACCGAGACCGTGAACACCCTGATCGAGC
AGAACGTGCCCGTGACCCAGGTGGAGGAGCTGGTGCACGGCGGCATCGACCCCGTGCTGTGCGGCACCGAG
CTGGGCTCCCCCCTGGTGCTGGACGACTGCTCCCTGGAGGGCCTGATCCTGGGCAACCCCAAGTGCGACCT
GTACCTGAACGGCCGCGAGTGGTCCTACATCGTGGAGCGCCCCAAGGAGATGGAGGGCGTGTGCTACCCCG
GCTCCATCGAGAACCAGGAGGAGCTGCGCTCCCTGTTCTCCTCCATCAAGAAGTACGAGCGCGTGAAGATG
TTCGACTTCACCAAGTGGAACGTGACCTACACCGGCACCTCCAAGGCCTGCAACAACACCTCCAACCAGGG
CTCCTTCTACCGCTCCATGCGCTGGCTGACCCTGAAGTCCGGCCAGTTCCCCGTGCAGACCGACGAGTACA
AGAACACCCGCGACTCCGACATCGTGTTCACCTGGGCCATCCACCACCCCCCCACCTCCGACGAGCAGATC
AAGCTGTACAAGAACCCCGACACCCTGTCCTCCGTGACCACCGACGAGATCAACCGCTCCTTCAAGCCCAA
CATCGGCCCCCGCCCCCTGGTGCGCGGCCAGCAGGGCCGCATGGACTACTACTGGGCCGTGCTGAAGCCCG
GCCAGACCGTGAAGATCCAGACCAACGGCAACCTGATCGCCCCCGAGTACGGCCACCTGATCACCGGCAAG
TCCCACGGCCGCATCCTGAAGAACAACCTGCCCATCGGCCAGTGCGTGACCGAGTGCCAGCTGAACGAGGG
CGTGATGAACACCTCCAAGCCCTTCCAGAACACCTCCAAGCACTACATCGGCAAGTGCCCCAAGTACATCC
CCTCCGGCTCCCTGAAGCTGGCCATCGGCCTGCGCAACGTGCCCCAGGTGCAGAACCGCGGCCTGTTCGGC
GCCAAGGCCGGCTTCATCGAGGGCGGCTGGCCCGGCCTGGTGGCCGGCTGGTACGGCTTCCAGCACCAGAA
CGCCGAGGGCACCGGCATCGCCGCCGACCGCGACTCCACCCAGAAGGCCATCGACAACATGCAGAACAAGC
TGAACAACGTGATCGACAAGATGAACAAGCAGTTCGAGGTGGTGAACCACGAGTTCTCCGAGGTGGAGTCC
CGCATCAACATGATCAACTCCCAGATCGACGACCAGATCACCGACATCTGGGCCTACAACGCCGAGCTGCT
GGTGCTGCTGGAGAACCAGAAGACCCTGGACGAGCACGACGCCAACGTGCGCAACCTGCACGACCGCGTGC
GCCGCGTGCTGCGCGAGAACGCCATCGACACCGGCGACGGCTGCTTCGAGATCCTGCACAAGTGCGACAAC
AACTGCATGGACACCATCCGCAACGGCACCTACAACCACAAGGAGTACGAGGAGGAGTCCAAGATCGAGCG
CCAGAAGATCAACGGCGTGAAGCTGGAGGAGAACTCCACCTACAAGATCCTGTCCATCTACTCCTCCGTGG
CCTCCTCCCTGGTGCTGCTGCTGATGATCATCGGCGGCTTCATCTTCGGCTGCCAGAACGGCAACGTGCGC
TGCACCTTCTGCATCtaa
2912_pUC-ccTEV-A-mallard-Sweden-24-02 HA-A101
(SEQ ID NO: 9)
ATGGCCATCTCCGGCGTGCCCGTGCTGGGCTTCTTCATCATCGCCGTGCTGATGTCCGCCCAGGAGTCCTG
GGCCTACGACCGCATCTGCATCGGCTACCAGTCCAACAACTCCACCGACACCGTGAACACCCTGATCGAGC
AGAACGTGCCCGTGACCCAGACCATGGAGCTGGTGGAGACCGAGAAGCACCCCGCCTACTGCAACACCGAC
CTGGGCGCCCCCCTGGAGCTGCGCGACTGCAAGATCGAGGCCGTGATCTACGGCAACCCCAAGTGCGACAT
CCACCTGAAGGACCAGGGCTGGTCCTACATCGTGGAGCGCCCCTCCGCCCCCGAGGGCATGTGCTACCCCG
GCTCCGTGGAGAACCTGGAGGAGCTGCGCTTCGTGTTCTCCTCCGCCGCCTCCTACAAGCGCATCCGCCTG
TTCGACTACTCCCGCTGGAACGTGACCCGCTCCGGCACCTCCAAGGCCTGCAACGCCTCCACCGGCGGCCA
GTCCTTCTACCGCTCCATCAACTGGCTGACCAAGAAGAAGCCCGACACCTACGACTTCAACGAGGGCACCT
ACATCAACAACGAGGACGGCGACATCATCTTCCTGTGGGGCATCCACCACCCCCCCGACGCCAAGGAGCAG
ACCACCCTGTACAAGAACGCCAACACCCTGTCCTCCGTGACCACCAACACCATCAACCGCTCCTTCCAGCC
CAACATCGGCCCCCGCCCCCTGGTGCGCGGCCAGCAGGGCCGCATGGACTACTACTGGGGCATCCTGAAGC
GCGGCGAGACCCTGAAGATCCGCACCAACGGCAACCTGATCGCCCCCGAGTTCGGCTACCTGCTGAAGGGC
GAGTCCCACGGCCGCATCATCCAGAACGAGGACATCCCCATCGGCTCCTGCCACACCAAGTGCCAGACCTA
CGCCGGCGCCATCAACTCCTCCAAGCCCTTCCAGAACGCCTCCCGCCACTACATGGGCGAGTGCCCCAAGT
ACGTGAAGAAGGAGTCCCTGCGCCTGGCCGTGGGCCTGCGCAACACCCCCTCCATCGAGCCCCGCGGCCTG
TTCGGCGCCATCGCCGGCTTCATCGAGGGCGGCTGGTCCGGCATGATCGACGGCTGGTACGGCTTCCACCA
CTCCAACTCCGAGGGCACCGGCATGGCCGCCGACCAGAAGTCCACCCAGGAGGCCATCGACAAGATCACCA
ACAAGGTGAACAACATCGTGGACAAGATGAACCGCGAGTTCGAGGTGGTGAACCACGAGTTCTCCGAGGTG
GAGAAGCGCATCAACATGATCAACGACAAGATCGACGACCAGATCGAGGACCTGTGGGCCTACAACGCCGA
GCTGCTGGTGCTGCTGGAGAACCAGAAGACCCTGGACGAGCACGACTCCAACGTGAAGAACCTGTTCGACG
AGGTGCGCCGCCGCCTGTCCGCCAACGCCATCGACACCGGCAACGGCTGCTTCGACATCCTGCACAAGTGC
GACAACGAGTGCATGGAGACCATCAAGAACGGCACCTACAACCACAAGGAGTACGAGGAGGAGGCCAAGCT
GGAGCGgTCCAAGATCAACGGCGTGAAGCTGGAGGAGAACACCACCTACAAGATCCTGTCCATCTACTCCA
CCGTGGCCGCCTCCCTGTGCCTGGCCATCCTGATCGCCGGCGGCCTGATCCTGGGCATGCAGAACGGCTCC
TGCCGCTGCATGTTCTGCATCtaa
2913_pUC-ccTEV-A-Michigan-45-2015 HA-A101
(SEQ ID NO: 10)
ATGGCCATCTCCGGCGTGCCCGTGCTGGGCTTCTTCATCATCGCCGTGCTGATGTCCGCCCAGGAGTCCTG
GGCCGACACCCTGTGCATCGGCTACCACGCCAACAACTCCACCGACACCGTGGACACCGTGCTGGAGAAGA
ACGTGACCGTGACCCACTCCGTGAACCTGCTGGAGGACAAGCACAACGGCAAGCTGTGCAAGCTGCGCGGC
GTGGCCCCCCTGCACCTGGGCAAGTGCAACATCGCCGGCTGGATCCTGGGCAACCCCGAGTGCGAGTCCCT
GTCCACCGCCTCCTCCTGGTCCTACATCGTGGAGACCTCCAACTCCGACAACGGCACCTGCTACCCCGGCG
ACTTCATCAACTACGAGGAGCTGCGCGAGCAGCTGTCCTCCGTGTCCTCCTTCGAGCGgTTCGAGATCTTC
CCCAAGACCTCCTCCTGGCCCAACCACGACTCCAACAAGGGCGTGACCGCCGCCTGCCCCCACGCCGGCGC
CAAGTCCTTCTACAAGAACCTGATCTGGCTGGTGAAGAAGGGCAACTCCTACCCCAAGCTGAACCAGTCCT
ACATCAACGACAAGGGCAAGGAGGTGCTGGTGCTGTGGGGCATCCACCACCCCTCCACCACCGCCGACCAG
CAGTCCCTGTACCAGAACGCCGACGCCTACGTGTTCGTGGGCACCTCCCGCTACTCCAAGAAGTTCAAGCC
CGAGATCGCCACCCGCCCCAAGGTGCGCGACCAGGAGGGCCGCATGAACTACTACTGGACCCTGGTGGAGC
CCGGCGACAAGATCACCTTCGAGGCCACCGGCAACCTGGTGGTGCCCCGCTACGCCTTCACCATGGAGCGC
AACGCCGGCTCCGGCATCATCATCTCCGACACCCCCGTGCACGACTGCAACACCACCTGCCAGACCCCCGA
GGGCGCCATCAACACCTCCCTGCCCTTCCAGAACATCCACCCCATCACCATCGGCAAGTGCCCCAAGTACG
TGAAGTCCACCAAGCTGCGCCTGGCCACCGGCCTGCGCAACGTGCCCTCCATCCAGTCCCGCGGCCTGTTC
GGCGCCATCGCCGGCTTCATCGAGGGCGGCTGGACCGGCATGGTGGACGGCTGGTACGGCTACCACCACCA
GAACGAGCAGGGCTCCGGCTACGCCGCCGACCTGAAGTCCACCCAGAACGCCATCGACAAGATCACCAACA
AGGTGAACTCCGTGATCGAGAAGATGAACACCCAGTTCACCGCCGTGGGCAAGGAGTTCAACCACCTGGAG
AAGCGCATCGAGAACCTGAACAAGAAGGTGGACGACGGCTTCCTGGACATCTGGACCTACAACGCCGAGCT
GCTGGTGCTGCTGGAGAACGAGCGCACCCTGGACTACCACGACTCCAACGTGAAGAACCTGTACGAGAAGG
TGCGCAACCAGCTGAAGAACAACGCCAAGGAGATCGGCAACGGCTGCTTCGAGTTCTACCACAAGTGCGAC
AACACCTGCATGGAGTCCGTGAAGAACGGCACCTACGACTACCCCAAGTACTCCGAGGAGGCCAAGCTGAA
CCGCGAGAAGATCGACGGCGTGAAGCTGGAGTCCACCCGCATCTACCAGATCCTGGCCATCTACTCCACCG
TGGCCTCCTCCCTGGTGCTGGTGGTGTCCCTGGGCGCCATCTCCTTCTGGATGTGCTCCAACGGCTCCCTG
CAGTGCCGCATCTGCATCtaa
2914_pUC-ccTEV-A-shearwater-West Australia-2576-79 HA-A101
(SEQ ID NO: 11)
ATGGCCATCTCCGGCGTGCCCGTGCTGGGCTTCTTCATCATCGCCGTGCTGATGTCCGCCCAGGAGTCCTG
GGCCGACAAGATCTGCCTGGGCCACCACGCCGTGGCCAACGGCACCAAGGTGAACACCCTGACCGAGCGCG
GCGTGGAGGTGGTGAACGCCACCGAGACCGTGGAGATCACCGGCATCGACAAGGTGTGCACCAAGGGCAAG
AAGGCCGTGGACCTGGGCTCCTGCGGCATCCTGGGCACCATCATCGGCCCCCCCCAGTGCGACCTGCACCT
GGAGTTCAAGGCCGACCTGATCATCGAGCGCCGCAACTCCTCCGACATCTGCTACCCCGGCCGCTTCACCA
ACGAGGAGGCCCTGCGCCAGATCATCCGCGAGTCCGGCGGCATCGACAAGGAGTCCATGGGCTTCCGCTAC
TCCGGCATCCGCACCGACGGCGCCACCTCCGCCTGCAAGCGCACCGTGTCCTCCTTCTACTCCGAGATGAA
GTGGCTGTCCTCCTCCATGAACAACCAGGTGTTCCCCCAGCTGAACCAGACCTACCGCAACACCCGCAAGG
AGCCCGCCCTGATCGTGTGGGGCGTGCACCACTCCTCCTCCCTGGACGAGCAGAACAAGCTGTACGGCACC
GGCAACAAGCTGATCACCGTGGGCTCCTCCAAGTACCAGCAGTCCTTCTCCCCCTCCCCCGGCGCCCGCCC
CAAGGTGAACGGCCAGGCCGGCCGCATCGACTTCCACTGGATGCTGCTGGACCCCGGCGACACCGTGACCT
TCACCTTCAACGGCGCCTTCATCGCCCCCGACCGCGCCACCTTCCTGCGCTCCAACGCCCCCTCCGGCATC
GAGTACAACGGCAAGTCCCTGGGCATCCAGTCCGACGCCCAGATCGACGAGTCCTGCGAGGGCGAGTGCTT
CTACTCCGGCGGCACCATCAACTCCCCCCTGCCCTTCCAGAACATCGACTCCCGCGCCGTGGGCAAGTGCC
CCCGCTACGTGAAGCAGTCCTCCCTGCCCCTGGCCCTGGGCATGAAGAACGTGCCCGAGAAGATCCGCACC
CGCGGCCTGTTCGGCGCCATCGCCGGCTTCATCGAGAACGGCTGGGAGGGCCTGATCGACGGCTGGTACGG
CTTCCGCCACCAGAACGCCCAGGGCCAGGGCACCGCCGCCGACTACAAGTCCACCCAGGCCGCCATCGACC
AGATCACCGGCAAGCTGAACCGCCTGATCGAGAAGACCAACAAGCAGTTCGAGCTGATCGACAACGAGTTC
ACCGAGGTGGAGCAGCAGATCGGCAACGTGATCAACTGGACCCGCGACTCCCTGACCGAGATCTGGTCCTA
CAACGCCGAGCTGCTGGTGGCCATGGAGAACCAGCACACCATCGACCTGGCCGACTCCGAGATGAACAAGC
TGTACGAGCGCGTGCGCCGCCAGCTGCGCGAGAACGCCGAGGAGGACGGCACCGGCTGCTTCGAGATCTTC
CACCGCTGCGACGACCAGTGCATGGAGTCCATCCGCAACAACACCTACAACCACACCGAGTACCGCCAGGA
GGCCCTGCAGAACCGCATCATGATCAACCCCGTGAAGCTGTCCTCCGGCTACAAGGACGTGATCCTGTGGT
TCTCCTTCGGCGCCTCCTGCGTGATGCTGCTGGCCATCGCCATGGGCCTGATCTTCATGTGCGTGAAGAAC
GGCAACCTGCGCTGCACCATCTGCATCtaa
2915_pUC-ccTEV-A-shorebird-Delaware-68-2004 HA-A101
(SEQ ID NO: 12)
ATGGCCATCTCCGGCGTGCCCGTGCTGGGCTTCTTCATCATCGCCGTGCTGATGTCCGCCCAGGAGTCCTG
GGCCGACCGCATCTGCGTGGGCTACCTGTCCACCAACTCCTCCGAGAAGGTGGACACCCTGCTGGAGAACG
GCGTGCCCGTGACCTCCTCCGTGGACCTGGTGGAGACCAACCACACCGGCACCTACTGCTCCCTGAACGGC
ATCTCCCCCGTGCACCTGGGCGACTGCTCCTTCGAGGGCTGGATCGTGGGCAACCCCTCCTGCGCCTCCAA
CCTGGGCATCCGCGAGTGGTCCTACCTGATCGAGGACCCCTCCGCCCCCCACGGCCTGTGCTACCCCGGCG
AGCTGGACAACAACGGCGAGCTGCGCCACCTGTTCTCCGGCATCAAGTCCTTCTCCCGCACCGAGCTGATC
GCCCCCACCTCCTGGGGCGCCGTGAACGACGGCGTGTCCTCCGCCTGCCAGGACAAGGGCGCCTCCTCCTT
CTACCGCAACCTGGTGTGGTTCGTGGAGCGCGGCAAGAAGTACCCCGTGATCCGCGGCACCTACAACAACA
CCACCGGCCGCGACGTGCTGGTGATGTGGGGCATCCACCACCCCGTGTCCGAGGACGAGGCCCGCAAGCTG
TACATCAACTCCAACCCCTACACCCTGGTGTCCACCGGCTCCTGGTCCAAGAAGTACAACCTGGAGATCGG
CATCCGCCCCGGCTACAACGGCCAGAAGTCCTGGATGAAGATCTACTGGTCCCTGATGCACCCCGGCGAGT
CCATCTCCTTCGAGTCCAACGGCGGCCTGCTGGCCCCCCGCTACGGCTACATCATCGAGGAGTACGGCAAG
GGCCGCATCTTCCAGTCCCGCATCCGCGCCGGCAAGTGCAACACCAAGTGCCAGACCTCCGTGGGCGGCAT
CAACACCAACAAGACCTTCCAGAACATCGAGCGCAACGCCCTGGGCGACTGCCCCAAGTACATCAAGTCCG
GCCAGCTGAAGCTGGCCACCGGCCTGCGCAACGTGCCCGCCATCGCCTCCCGCGGCCTGTTCGGCGCCATC
GCCGGCTTCATCGAGGGCGGCTGGCCCGGCCTGATCAACGGCTGGTACGGCTTCCAGCACCAGAACGAGCA
GGGCGTGGGCATCGCCGCCGACAAGGAGTCCACCCAGAAGGCCATCGACCAGATCACCACCAAGATCAACA
ACATCATCGAGAAGATGAACGGCAACTACGACTCCATCCGCGGCGAGTTCAACCAGGTGGAGAACCGCATC
AACATGCTGGCCGACCGCATCGACGACGCCGTGACCGACGTGTGGTCCTACAACGCCAAGCTGCTGGTGCT
GCTGGAGAACGACAAGACCCTGGACATGCACGACGCCAACGTGCGCAACCTGCACGACCAGGTGCGCCGCG
CCCTGAAGACCAACGCCATCGACGAGGGCAACGGCTGCTTCGAGCTGCTGCACAAGTGCAACGACTCCTGC
ATGGAGACCATCCGCAACGGCACCTACAACCACACCGAGTACGAGGAGGAGTCCAAGCTGAAGCGCCAGGA
GATCGAGGGCATCAAGCTGAAGTCCGAGGACGGCGTGTACAAGGCCCTGTCCATCTACTCCTGCATCGCCT
CCTCCGTGGTGCTGGTGGGCCTGATCCTGGCCTTCATCATGTGGGCCTGCAACTCCGGCAACTGCCGCTTC
AACATCTGCATCtaa
2916_pUC-ccTEV-shoveler-Netherlands-18-99 HA-A101
(SEQ ID NO: 13)
ATGGCCATCTCCGGCGTGCCCGTGCTGGGCTTCTTCATCATCGCCGTGCTGATGTCCGCCCAGGAGTCCTG
GGCCGACGAGATCTGCATCGGCTACCTGTCCAACAACTCCACCGACAAGGTGGACACCATCATCGAGAACA
ACGTGACCGTGACCTCCTCCGTGGAGCTGGTGGAGACCGAGCACACCGGCTCCTTCTGCTCCATCAACGGC
AAGCAGCCCATCTCCCTGGGCGACTGCTCCTTCGCCGGCTGGATCCTGGGCAACCCCATGTGCGACGACCT
GATCGGCAAGACCTCCTGGTCCTACATCGTGGAGAAGCCCAACCCCACCAACGGCATCTGCTACCCCGGCA
CCCTGGAGGACGAGGAGGAGCTGCGCCTGAAGTTCTCCGGCGTGCTGGAGTTCTCCAAGTTCGAGGCCTTC
ACCTCCAACGGCTGGGGCGCCGTGAACTCCGGCGCCGGCGTGACCGCCGCCTGCAAGTTCGGCTCCTCCAA
CTCCTTCTTCCGCAACATGGTGTGGCTGATCCACCAGTCCGGCACCTACCCCGTGATCAAGCGCACCTTCA
ACAACACCAAGGGCCGCGACGTGCTGATCGTGTGGGGCATCCACCACCCCGCCACCCTGAAGGAGCACCAG
GACCTGTACAAGAAGGACTCCTCCTACGTGGCCGTGGGCTCCGAGACCTACAACCGCCGCTTCACCCCCGA
GATCTCCACCCGCCCCAAGGTGAACGGCCAGGCCGGCCGCATGACCTTCTACTGGACCATGGTGAAGCCCG
GCGAGTCCATCACCTTCGAGTCCAACGGCGCCTTCCTGGCCCCCCGCTACGCCTTCGAGATCGTGTCCGTG
GGCAACGGCAAGCTGTTCCGCTCCGAGCTGTCCATCGAGTCCTGCTCCACCAAGTGCCAGACCGAGGTGGG
CGGCATCAACACCAACAAGTCCTTCCACTCCGTGCACCGCAACACCATCGGCGACTGCCCCAAGTACGTGA
ACGTGAAGTCCCTGAAGCTGGCCACCGGCCTGCGCAACGTGCCCGCCATCGCCTCCCGCGGCCTGTTCGGC
GCCATCGCCGGCTTCATCGAGGGCGGCTGGCCCGGCCTGATCAACGGCTGGTACGGCTTCCAGCACCGCAA
CGAGGAGGGCACCGGCATCGCCGCCGACCGCGAGTCCACCCAGAAGGCCGTGGACCAGATCACCTCCAAGG
TGAACAACATCGTGGACCGCATGAACACCAACTTCGAGTCCGTGCAGCACGAGTTCTCCGAGATCGAGGAG
CGCATCAACCAGCTGTCCAAGCACGTGGACGACTCCGTGGTGGACATCTGGTCCTACAACGCCCAGCTGCT
GGTGTGGCTGGAGAACGAGAAGACCCTGGACCTGCACGACTCCAACGTGCGCAACCTGCACGAGAAGGTGC
GCCGCATGCTGAAGGACAACGCCAAGGACGAGGGCAACGGCTGCTTCACCTTCTACCACAAGTGCGACAAC
GAGTGCATCGAGAAGGTGCGCAACGGCACCTACGACCACAAGGAGTTCGAGGAGGAGTCCCGCATCAACCG
CCAGGAGATCGAGGGCGCCCGCCTGGACTCCTCCGGCAACGTGTACAAGATCCTGTCCATCTGCTCCTGCA
TCGCCTCCTCCCTGGTGCTGGCCGCCCTGATCATGGGCTTCATCCTGTGGGCCTGCTCCAACGGCTCCTGC
CGCTGCACCATCTGCATCtaa
2917_pUC-ccTEV-A-Singapore-INFIMH-16-0019-2016 HA-A101
(SEQ ID NO: 14)
ATGGCCATCTCCGGCGTGCCCGTGCTGGGCTTCTTCATCATCGCCGTGCTGATGTCCGCCCAGGAGTCCTG
GGCCCAGAAGATCCCCGGCAACGACAACTCCACCGCCACCCTGTGCCTGGGCCACCACGCCGTGCCCAACG
GCACCATCGTGAAGACCATCACCAACGACCGCATCGAGGTGACCAACGCCACCGAGCTGGTGCAGAACTCC
TCCATCGGCGAGATCTGCGACTCCCCCCACCAGATCCTGGACGGCGAGAACTGCACCCTGATCGACGCCCT
GCTGGGCGACCCCCAGTGCGACGGCTTCCAGAACAAGAAGTGGGACCTGTTCGTGGAGCGgTCCAAGGCCT
ACTCCAACTGCTACCCCTACGACGTGCCCGACTACGCCTCCCTGCGCTCCCTGGTGGCCTCCTCCGGCACC
CTGGAGTTCAAGAACGAGTCCTTCAACTGGACCGGCGTGACCCAGAACGGCACCTCCTCCGCCTGCATCCG
CGGCTCCTCCTCCTCCTTCTTCTCCCGCCTGAACTGGCTGACCCACCTGAACTACACCTACCCCGCCCTGA
ACGTGACCATGCCCAACAAGGAGCAGTTCGACAAGCTGTACATCTGGGGCGTGCACCACCCCGGCACCGAC
AAGGACCAGATCTTCCTGTACGCCCAGTCCTCCGGCCGCATCACCGTGTCCACCAAGCGgTCCCAGCAGGC
CGTGATCCCCAACATCGGCTCCCGCCCCCGCATCCGCGACATCCCCTCCCGCATCTCCATCTACTGGACCA
TCGTGAAGCCCGGCGACATCCTGCTGATCAACTCCACCGGCAACCTGATCGCCCCCCGCGGCTACTTCAAG
ATCCGCTCCGGCAAGTCCTCCATCATGCGCTCCGACGCCCCCATCGGCAAGTGCAAGTCCGAGTGCATCAC
CCCCAACGGCTCCATCCCCAACGACAAGCCCTTCCAGAACGTGAACCGCATCACCTACGGCGCCTGCCCCC
GCTACGTGAAGCACTCCACCCTGAAGCTGGCCACCGGCATGCGCAACGTGCCCGAGAAGCAGACCCGCGGC
ATCTTCGGCGCCATCGCCGGCTTCATCGAGAACGGCTGGGAGGGCATGGTGGACGGCTGGTACGGCTTCCG
CCACCAGAACTCCGAGGGCCGCGGCCAGGCCGCCGACCTGAAGTCCACCCAGGCCGCCATCGACCAGATCA
ACGGCAAGCTGAACCGCCTGATCGGCAAGACCAACGAGAAGTTCCACCAGATCGAGAAGGAGTTCTCCGAG
GTGGAGGGCCGCGTGCAGGACCTGGAGAAGTACGTGGAGGACACCAAGATCGACCTGTGGTCCTACAACGC
CGAGCTGCTGGTGGCCCTGGAGAACCAGCACACCATCGACCTGACCGACTCCGAGATGAACAAGCTGTTCG
AGAAGACCAAGAAGCAGCTGCGCGAGAACGCCGAGGACATGGGCAACGGCTGCTTCAAGATCTACCACAAG
TGCGACAACGCCTGCATCGAGTCCATCCGCAACGAGACCTACGACCACAACGTGTACCGCGACGAGGCCCT
GAACAACCGCTTCCAGATCAAGGGCGTGGAGCTGAAGTCCGGCTACAAGGACTGGATCCTGTGGATCTCCT
TCGCCATCTCCTGCTTCCTGCTGTGCGTGGCCCTGCTGGGCTTCATCATGTGGGCCTGCCAGAAGGGCAAC
ATCCGCTGCAACATCTGCATCtaa
2918_pUC-ccTEV-A-Taiwan-2-2013 HA-A101
(SEQ ID NO: 15)
ATGGCCATCTCCGGCGTGCCCGTGCTGGGCTTCTTCATCATCGCCGTGCTGATGTCCGCCCAGGAGTCCTG
GGCCGACAAGATCTGCATCGGCTACCACGCCAACAACTCCACCACCCAGGTGGACACCCTGCTGGAGAAGA
ACGTGACCGTGACCCACTCCGTGGAGCTGCTGGAGAACCAGAAGGAGAAGCGgTTCTGCAAGATCATGAAC
AAGGCCCCCCTGGACCTGAAGGACTGCACCATCGAGGGCTGGATCCTGGGCAACCCCAAGTGCGACCTGCT
GCTGGGCGACCAGTCCTGGTCCTACATCGTGGAGCGCCCCAACGCCCAGAACGGCATCTGCTACCCCGGCG
TGCTGAACGAGCTGGAGGAGCTGAAGGCCTTCATCGGCTCCGGCGAGCGCGTGGAGCGgTTCGAGATGTTC
CCCAAGTCCACCTGGGCCGGCGTGGACACCTCCCGCGGCGTGACCAACGCCTGCCCCTCCTACACCATCGA
CTCCTCCTTCTACCGCAACCTGGTGTGGATCGTGAAGACCGACTCCGCCACCTACCCCGTGATCAAGGGCA
CCTACAACAACACCGGCACCCAGCCCATCCTGTACTTCTGGGGCGTGCACCACCCCCTGGACACCACCGTG
CAGGACAACCTGTACGGCTCCGGCGACAAGTACGTGCGCATGGGCACCGAGTCCATGAACTTCGCCAAGTC
CCCCGAGATCGCCGCCCGCCCCGCCGTGAACGGCCAGCGgTCCCGCATCGACTACTACTGGTCCGTGCTGC
GCCCCGGCGAGACCCTGAACGTGGAGTCCAACGGCAACCTGATCGCCCCCTGGTACGCCTACAAGTTCGTG
TCCACCAACAAGAAGGGCGCCGTGTTCAAGTCCGACCTGCCCATCGAGAACTGCGACGCCACCTGCCAGAC
CATCACCGGCGTGCTGCGCACCAACAAGACCTTCCAGAACGTGTCCCCCCTGTGGATCGGCGAGTGCCCCA
AGTACGTGAAGTCCGAGTCCCTGCGCCTGGCCACCGGCCTGCGCAACGTGCCCCAGATCGCCACCCGCGGC
ATCTTCGGCGCCATCGCCGGCTTCATCGAGGGCGGCTGGACCGGCATGATCGACGGCTGGTACGGCTACCA
CCACGAGAACTCCCAGGGCTCCGGCTACGCCGCCGACCGCGAGTCCACCCAGAAGGCCATCGACGGCATCA
CCAACAAGGTGAACTCCATCATCAACAAGATGAACACCCAGTTCGAGGCCGTGGACCACGAGTTCTCCAAC
CTGGAGCGCCGCATCGGCAACCTGAACAAGCGCATGGAGGACGGCTTCCTGGACGTGTGGACCTACAACGC
CGAGCTGCTGGTGCTGCTGGAGAACGAGCGCACCCTGGACCTGCACGACGCCAACGTGAAGAACCTGTACG
AGAAGGTGAAGTCCCAGCTGCGCGACAACGCCAACGACCTGGGCAACGGCTGCTTCGAGTTCTGGCACAAG
TGCGACAACGAGTGCATGGAGTCCGTGAAGAACGGCACCTACGACTACCCCAAGTACCAGAAGGAGTCCAA
GCTGAACCGCCAGGGCATCGAGTCCGTGAAGCTGGAGAACCTGGGCGTGTACCAGATCCTGGCCATCTACT
CCACCGTGTCCTCCTCCCTGGTGCTGGTGGGCCTGATCATGGCCATCGGCCTGTGGATGTGCTCCAACGGC
TCCATGCAGTGCCGCATCTGCATCtaa
2919_pUC-ccTEV-A-yellow shouldered bat-Guatemala-06-10 HA-A101
(SEQ ID NO: 16)
ATGGCCATCTCCGGCGTGCCCGTGCTGGGCTTCTTCATCATCGCCGTGCTGATGTCCGCCCAGGAGTCCTG
GGCCGACCGCATCTGCATCGGCTACCAGGCCAACCAGAACAACCAGACCGTGAACACCCTGCTGGAGCAGA
ACGTGCCCGTGACCGGCGCCCAGGAGATCCTGGAGACCAACCACAACGGCAAGCTGTGCTCCCTGAACGGC
GTGCCCCCCCTGGACCTGCAGTCCTGCACCCTGGCCGGCTGGCTGCTGGGCAACCCCAACTGCGACAACCT
GCTGGAGGCCGAGGAGTGGTCCTACATCAAGATCAACGAGAACGCCCCCGACGACCTGTGCTTCCCCGGCA
ACTTCGAGAACCTGCAGGACCTGCTGCTGGAGATGTCCGGCGTGCAGAACTTCACCAAGGTGAAGCTGTTC
AACCCCCAGTCCATGACCGGCGTGACCACCAACAACGTGGACCAGACCTGCCCCTTCGAGGGCAAGCCCTC
CTTCTACCGCAACCTGAACTGGATCCAGGGCAACTCCGGCCTGCCCTTCAACATCGAGATCAAGAACCCCA
CCTCCAACCCCCTGCTGCTGCTGTGGGGCATCCACAACACCAAGGACGCCGCCCAGCAGCGCAACCTGTAC
GGCAACGACTACTCCTACACCATCTTCAACTTCGGCGAGAAGTCCGAGGAGTTCCGCCCCGACATCGGCCA
GCGCGACGAGATCAAGGCCCACCAGGACCGCATCGACTACTACTGGGGCTCCCTGCCCGCCCAGTCCACCC
TGCGCATCGAGTCCACCGGCAACCTGATCGCCCCCGAGTACGGCTTCTACTACAAGCGCAAGGAGGGCAAG
GGCGGCCTGATGAAGTCCAAGCTGCCCATCTCCGACTGCTCCACCAAGTGCCAGACCCCCCTGGGCGCCCT
GAACTCCACCCTGCCCTTCCAGAACGTGCACCAGCAGACCATCGGCAACTGCCCCAAGTACGTGAAGGCCA
CCTCCCTGATGCTGGCCACCGGCCTGCGCAACAACCCCCAGATGGAGGGCCGCGGCCTGTTCGGCGCCATC
GCCGGCTTCATCGAGGGCGGCTGGCAGGGCATGATCGACGGCTGGTACGGCTACCACCACGAGAACCAGGA
GGGCTCCGGCTACGCCGCCGACAAGGAGGCCACCCAGAAGGCCGTGGACGCCATCACCAACAAGGTGAACT
CCATCATCGACAAGATGAACTCCCAGTTCGAGTCCAACATCAAGGAGTTCAACCGCCTGGAGCTGCGCATC
CAGCACCTGTCCGACCGCGTGGACGACGCCCTGCTGGACATCTGGTCCTACAACACCGAGCTGCTGGTGCT
GCTGGAGAACGAGCGCACCCTGGACTTCCACGACGCCAACGTGAAGAACCTGTTCGAGAAGGTGAAGGCCC
AGCTGAAGGACAACGCCATCGACGAGGGCAACGGCTGCTTCCTGCTGCTGCACAAGTGCAACAACTCCTGC
ATGGACGACATCAAGAACGGCACCTACAAGTACATGGACTACCGCGAGGAGTCCCACATCGAGAAGCAGAA
GATCGACGGCGTGAAGCTGACCGACTACTCCCGCTACTACACCATGACCCTGTACTCCACCATCGCCTCCT
CCGTGGTGCTGGGCTCCCTGATCATCGCCGCCTTCCTGTGGGGCTGCCAGAAGGGCTCCATCCAGTGCAAG
ATCTGCATCtaa
2920_pUC-ccTEV-B-Colorado-06-2017 HA-A101
(SEQ ID NO: 17)
ATGGCCATCTCCGGCGTGCCCGTGCTGGGCTTCTTCATCATCGCCGTGCTGATGTCCGCCCAGGAGTCCTG
GGCCGACCGCATCTGCACCGGCATCACCTCCTCCAACTCCCCCCACGTGGTGAAGACCGCCACCCAGGGCG
AGGTGAACGTGACCGGCGTGATCCCCCTGACCACCACCCCCACCAAGTCCCACTTCGCCAACCTGAAGGGC
ACCGAGACCCGCGGCAAGCTGTGCCCCAAGTGCCTGAACTGCACCGACCTGGACGTGGCCCTGGGCCGCCC
CAAGTGCACCGGCAAGATCCCCTCCGCCCGCGTGTCCATCCTGCACGAGGTGCGCCCCGTGACCTCCGGCT
GCTTCCCCATCATGCACGACCGCACCAAGATCCGCCAGCTGCCCAACCTGCTGCGCGGCTACGAGCACGTG
CGCCTGTCCACCCACAACGTGATCAACGCCGAGGGCGCCCCCGGCGGCCCCTACAAGATCGGCACCTCCGG
CTCCTGCCCCAACATCACCAACGGCAACGGCTTCTTCGCCACCATGGCCTGGGCCGTGCCCGACAAGAACA
AGACCGCCACCAACCCCCTGACCATCGAGGTGCCCTACGTGTGCACCGAGGGCGAGGACCAGATCACCGTG
TGGGGCTTCCACTCCGACAACGAGACCCAGATGGCCAAGCTGTACGGCGACTCCAAGCCCCAGAAGTTCAC
CTCCTCCGCCAACGGCGTGACCACCCACTACGTGTCCCAGATCGGCGGCTTCCCCAACCAGACCGAGGACG
GCGGCCTGCCCCAGTCCGGCCGCATCGTGGTGGACTACATGGTGCAGAAGTCCGGCAAGACCGGCACCATC
ACCTACCAGCGCGGCATCCTGCTGCCCCAGAAGGTGTGGTGCGCCTCCGGCCGCTCCAAGGTGATCAAGGG
CTCCCTGCCCCTGATCGGCGAGGCCGACTGCCTGCACGAGAAGTACGGCGGCCTGAACAAGTCCAAGCCCT
ACTACACCGGCGAGCACGCCAAGGCCATCGGCAACTGCCCCATCTGGGTGAAGACCCCCCTGAAGCTGGCC
AACGGCACCAAGTACCGCCCCCCCGCCAAGCTGCTGAAGGAGCGCGGCTTCTTCGGCGCCATCGCCGGCTT
CCTGGAGGGCGGCTGGGAGGGCATGATCGCCGGCTGGCACGGCTACACCTCCCACGGCGCCCACGGCGTGG
CCGTGGCCGCCGACCTGAAGTCCACCCAGGAGGCCATCAACAAGATCACCAAGAACCTGAACTCCCTGTCC
GAGCTGGAGGTGAAGAACCTGCAGCGCCTGTCCGGCGCCATGGACGAGCTGCACAACGAGATCCTGGAGCT
GGACGAGAAGGTGGACGACCTGCGCGCCGACACCATCTCCTCCCAGATCGAGCTGGCCGTGCTGCTGTCCA
ACGAGGGCATCATCAACTCCGAGGACGAGCACCTGCTGGCCCTGGAGCGCAAGCTGAAGAAGATGCTGGGC
CCCTCCGCCGTGGAGATCGGCAACGGCTGCTTCGAGACCAAGCACAAGTGCAACCAGACCTGCCTGGACAA
GATCGCCGCCGGCACCTTCGACGCCGGCGAGTTCTCCCTGCCCACCTTCGACTCCCTGAACATCACCGCCG
CCTCCCTGAACGACGACGGCCTGGACAACCACACCATCCTGCTGTACTACTCCACCGCCGCCTCCTCCCTG
GCCGTGACCCTGATGATCGCCATCTTCGTGGTGTACATGGTGTCCCGCGACAACGTGTCCTGCTCCATCTG
CCTGtaa
2921_pUC-ccTEV-B-Phuket-3073-2013 HA-A101
(SEQ ID NO: 18)
ATGGCCATCTCCGGCGTGCCCGTGCTGGGCTTCTTCATCATCGCCGTGCTGATGTCCGCCCAGGAGTCCTG
GGCCGACCGCATCTGCACCGGCATCACCTCCTCCAACTCCCCCCACGTGGTGAAGACCGCCACCCAGGGCG
AGGTGAACGTGACCGGCGTGATCCCCCTGACCACCACCCCCACCAAGTCCTACTTCGCCAACCTGAAGGGC
ACCCGCACCCGCGGCAAGCTGTGCCCCGACTGCCTGAACTGCACCGACCTGGACGTGGCCCTGGGCCGCCC
CATGTGCGTGGGCACCACCCCCTCCGCCAAGGCCTCCATCCTGCACGAGGTGCGCCCCGTGACCTCCGGCT
GCTTCCCCATCATGCACGACCGCACCAAGATCCGCCAGCTGCCCAACCTGCTGCGCGGCTACGAGAAGATC
CGCCTGTCCACCCAGAACGTGATCGACGCCGAGAAGGCCCCCGGCGGCCCCTACCGCCTGGGCACCTCCGG
CTCCTGCCCCAACGCCACCTCCAAGATCGGCTTCTTCGCCACCATGGCCTGGGCCGTGCCCAAGGACAACT
ACAAGAACGCCACCAACCCCCTGACCGTGGAGGTGCCCTACATCTGCACCGAGGGCGAGGACCAGATCACC
GTGTGGGGCTTCCACTCCGACAACAAGACCCAGATGAAGTCCCTGTACGGCGACTCCAACCCCCAGAAGTT
CACCTCCTCCGCCAACGGCGTGACCACCCACTACGTGTCCCAGATCGGCGACTTCCCCGACCAGACCGAGG
ACGGCGGCCTGCCCCAGTCCGGCCGCATCGTGGTGGACTACATGATGCAGAAGCCCGGCAAGACCGGCACC
ATCGTGTACCAGCGCGGCGTGCTGCTGCCCCAGAAGGTGTGGTGCGCCTCCGGCCGCTCCAAGGTGATCAA
GGGCTCCCTGCCCCTGATCGGCGAGGCCGACTGCCTGCACGAGGAGTACGGCGGCCTGAACAAGTCCAAGC
CCTACTACACCGGCAAGCACGCCAAGGCCATCGGCAACTGCCCCATCTGGGTGAAGACCCCCCTGAAGCTG
GCCAACGGCACCAAGTACCGCCCCCCCGCCAAGCTGCTGAAGGAGCGCGGCTTCTTCGGCGCCATCGCCGG
CTTCCTGGAGGGCGGCTGGGAGGGCATGATCGCCGGCTGGCACGGCTACACCTCCCACGGCGCCCACGGCG
TGGCCGTGGCCGCCGACCTGAAGTCCACCCAGGAGGCCATCAACAAGATCACCAAGAACCTGAACTCCCTG
TCCGAGCTGGAGGTGAAGAACCTGCAGCGCCTGTCCGGCGCCATGGACGAGCTGCACAACGAGATCCTGGA
GCTGGACGAGAAGGTGGACGACCTGCGCGCCGACACCATCTCCTCCCAGATCGAGCTGGCCGTGCTGCTGT
CCAACGAGGGCATCATCAACTCCGAGGACGAGCACCTGCTGGCCCTGGAGCGCAAGCTGAAGAAGATGCTG
GGCCCCTCCGCCGTGGACATCGGCAACGGCTGCTTCGAGACCAAGCACAAGTGCAACCAGACCTGCCTGGA
CCGCATCGCCGCCGGCACCTTCAACGCCGGCGAGTTCTCCCTGCCCACCTTCGACTCCCTGAACATCACCG
CCGCCTCCCTGAACGACGACGGCCTGGACAACCACACCATCCTGCTGTACTACTCCACCGCCGCCTCCTCC
CTGGCCGTGACCCTGATGCTGGCCATCTTCATCGTGTACATGGTGTCCCGCGACAACGTGTCCTGCTCCAT
CTGCCTGtaa
3525_pUC-ccTEV-A_Vietnam 1203_2004 HA-A101
(SEQ ID NO: 19)
ATGGAGAAGATCGTGCTGCTGTTCGCCATCGTGTCCCTGGTGAAGTCCGACCAGATCTGCATCGGCTACCA
CGCCAACAACTCCACCGAGCAGGTGGACACCATCATGGAGAAGAACGTGACCGTGACCCACGCCCAGGACA
TCCTGGAGAAGAAGCACAACGGCAAGCTGTGCGACCTGGACGGCGTGAAGCCCCTGATCCTGCGCGACTGC
TCCGTGGCCGGCTGGCTGCTGGGCAACCCCATGTGCGACGAGTTCATCAACGTGCCCGAGTGGTCCTACAT
CGTGGAGAAGGCCAACCCCGTGAACGACCTGTGCTACCCCGGCGACTTCAACGACTACGAGGAGCTGAAGC
ACCTGCTGTCCCGCATCAACCACTTCGAGAAGATCCAGATCATCCCCAAGTCCTCCTGGTCCTCCCACGAG
GCCTCCCTGGGCGTGTCCTCCGCCTGCCCCTACCAGGGCAAGTCCTCCTTCTTCCGCAACGTGGTGTGGCT
GATCAAGAAGAACTCCACCTACCCCACCATCAAaCGCTCCTACAACAACACCAACCAGGAGGACCTGCTGG
TGCTGTGGGGCATCCACCACCCCAACGACGCCGCCGAGCAGACCAAGCTGTACCAGAACCCCACCACCTAC
ATCTCCGTGGGCACCTCCACCCTGAACCAGCGCCTGGTGCCCCGCATCGCCACCCGCTCCAAGGTGAACGG
CCAGTCCGGCCGCATGGAGTTCTTCTGGACCATCCTGAAGCCCAACGACGCCATCAACTTCGAGTCCAACG
GCAACTTCATCGCCCCCGAGTACGCCTACAAGATCGTGAAGAAGGGCGACTCCACCATCATGAAGTCCGAG
CTGGAGTACGGCAACTGCAACACCAAGTGCCAGACCCCCATGGGCGCCATCAACTCCTCCATGCCCTTCCA
CAACATCCACCCCCTGACCATCGGCGAGTGCCCCAAGTACGTGAAGTCCAACCGCCTGGTGCTGGCCACCG
GCCTGCGCAACTCCCCCCAGCGCGAGCGCCGCCGCAAGAAGCGCGGCCTGTTCGGCGCCATCGCCGGCTTC
ATCGAGGGCGGCTGGCAGGGCATGGTGGACGGCTGGTACGGCTACCACCACTCCAACGAGCAGGGCTCCGG
CTACGCCGCCGACAAGGAGTCCACCCAGAAGGCCATCGACGGCGTGACCAACAAGGTGAACTCCATCATCG
ACAAGATGAACACCCAGTTCGAGGCCGTGGGCCGCGAGTTCAACAACCTGGAGCGCCGCATCGAGAACCTG
AACAAGAAGATGGAGGACGGCTTCCTGGACGTGTGGACCTACAACGCCGAGCTGCTGGTGCTGATGGAGAA
CGAGCGCACCCTGGACTTCCACGACTCCAACGTGAAGAACCTGTACGACAAGGTGCGCCTGCAGCTGCGCG
ACAACGCCAAGGAGCTGGGCAACGGCTGCTTCGAGTTCTACCACAAGTGCGACAACGAGTGCATGGAGTCC
GTGCGCAACGGCACCTACGACTACCCCCAGTACTCCGAGGAGGCCCGCCTGAAGCGCGAGGAGATCTCCGG
CGTGAAGCTGGAGTCCATCGGCATCTACCAGATCCTGTCCATCTACTCCACCGTGGCCTCCTCCCTGGCCC
TGGCCATCATGGTGGCCGGCCTGTCCCTGTGGATGTGCTCCAACGGCTCCCTGCAGTGCCGCATCTGCATC
taa
3526_pUC-ccTEV-A_Shanghai_02_2013 HA-A101
(SEQ ID NO: 20)
ATGAACACCCAGATCCTGGTGTTCGCCCTGATCGCCATCATCCCCACCAACGCCGACAAGATCTGCCTGGG
CCACCACGCCGTGTCCAACGGCACCAAGGTGAACACCCTGACCGAGCGCGGCGTGGAGGTGGTGAACGCCA
CCGAGACCGTGGAGCGCACCAACATCCCCCGCATCTGCTCCAAGGGCAAGCGCACCGTGGACCTGGGCCAG
TGCGGCCTGCTGGGCACCATCACCGGCCCCCCCCAGTGCGACCAGTTCCTGGAGTTCTCCGCCGACCTGAT
CATCGAGCGCCGCGAGGGCTCCGACGTGTGCTACCCCGGCAAGTTCGTGAACGAGGAGGCCCTGCGCCAGA
TCCTGCGCGAGTCCGGCGGCATCGACAAGGAGGCCATGGGCTTCACCTACTCCGGCATCCGCACCAACGGC
GCCACCTCCGCCTGCCGCCGCTCCGGCTCCTCCTTCTACGCCGAGATGAAGTGGCTGCTGTCCAACACCGA
CAACGCCGCCTTCCCCCAGATGACCAAGTCCTACAAGAACACCCGCAAGTCCCCCGCCCTGATCGTGTGGG
GCATCCACCACTCCGTGTCCACCGCCGAGCAGACCAAGCTGTACGGCTCCGGCAACAAGCTGGTGACCGTG
GGCTCCTCCAACTACCAGCAGTCCTTCGTGCCCTCCCCCGGCGCCCGCCCCCAGGTGAACGGCCTGTCCGG
CCGCATCGACTTCCACTGGCTGATGCTGAACCCCAACGACACCGTGACCTTCTCCTTCAACGGCGCCTTCA
TCGCCCCCGACCGCGCCTCCTTCCTGCGCGGCAAGTCCATGGGCATCCAGTCCGGCGTGCAGGTGGACGCC
AACTGCGAGGGCGACTGCTACCACTCCGGCGGCACCATCATCTCCAACCTGCCCTTCCAGAACATCGACTC
CCGCGCCGTGGGCAAGTGCCCCCGCTACGTGAAGCAGCGgTCCCTGCTGCTGGCCACCGGCATGAAGAACG
TGCCCGAGATCCCCAAGGGCCGCGGCCTGTTCGGCGCCATCGCCGGCTTCATCGAGAACGGCTGGGAGGGC
CTGATCGACGGCTGGTACGGCTTCCGCCACCAGAACGCCCAGGGCGAGGGCACCGCCGCCGACTACAAGTC
CACCCAGTCCGCCATCGACCAGATCACCGGCAAGCTGAACCGCCTGATCGAGAAGACCAACCAGCAGTTCG
AGCTGATCGACAACGAGTTCAACGAGGTGGAGAAGCAGATCGGCAACGTGATCAACTGGACCCGCGACTCC
ATCACCGAGGTGTGGTCCTACAACGCCGAGCTGCTGGTGGCCATGGAGAACCAGCACACCATCGACCTGGC
CGACTCCGAGATGGACAAGCTGTACGAGCGCGTGAAGCGCCAGCTGCGCGAGAACGCCGAGGAGGACGGCA
CCGGCTGCTTCGAGATCTTCCACAAGTGCGACGACGACTGCATGGCCTCCATCCGCAACAACACCTACGAC
CACTCCAAGTACCGCGAGGAGGCCATGCAGAACCGCATCCAGATCGACCCCGTGAAGCTGTCCTCCGGCTA
CAAGGACGTGATCCTGTGGTTCTCCTTCGGCGCCTCCTGCTTCATCCTGCTGGCCATCGTGATGGGCCTGG
TGTTCATCTGCGTGAAGAACGGCAACATGCGCTGCACCATCTGCATCtaa

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for inducing an immune response against one or more influenza viruses in a subject, the composition comprising a combination of at least two lipid nanoparticle (LNPs) comprising a combination of nucleoside-modified RNA molecules encoding at least two influenza virus antigens, wherein the combination of at least two nucleoside-modified RNA molecules encode hemagglutinin (HA) antigens, or fragments thereof, are selected from the group consisting of influenza A virus H1, H2, H3, H4, H5, H6, H7, H8, H9, H10, H11, H12, H13, H14, H15, H16, H17, and H18, and influenza B virus Vic and Yam.

2. The composition of claim 1, wherein the HA antigen, or fragment thereof, is selected from the group consisting of a full-length HA antigen or a fragment thereof, HA-stalk domain or a fragment thereof, HA-head domain or a fragment thereof, and any combination thereof.

3. The composition of claim 1, comprising nucleoside-modified RNA molecules encoding HA antigens from each of influenza A virus H1, H2, H3, H4, H5, H6, H7, H8, H9, H10, H11, H12, H13, H14, H15, H16, H17, and H18, and influenza B virus Vic and Yam.

4. The composition of claim 1, further comprising at least one additional viral antigen.

5. The composition of claim 1, wherein the composition comprises a combination of at least two mRNA molecules encoded by nucleotide sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

6. The composition of claim 1, wherein the composition further comprises an adjuvant.

7. The composition of claim 1, wherein the nucleoside-modified RNA molecules are encapsulated within the LNPs.

8. The composition of claim 1, wherein the nucleoside-modified RNA molecules comprise at least one modified nucleoside selected from the group consisting of pseudouridine, 1-methyl pseudouridine, and 5-methyl-uridine.

9. The composition of claim 1, wherein the composition is a universal influenza vaccine.

10. A method of inducing an immune response against multiple strains of influenza virus in a subject comprising administering to the subject an effective amount of a composition comprising a combination of at least two lipid nanoparticle (LNPs) wherein each LNP comprises a nucleoside-modified RNA encoding at least one influenza virus antigen or a fragment thereof, and further wherein the combination of at least two LNPs together comprise at least two nucleoside-modified RNA molecule encoding hemagglutinin (HA) antigens, or fragments thereof selected from the group consisting of influenza A virus H1, H2, H3, H4, H5, H6, H7, H8, H9, H10, H11, H12, H13, H14, H15, H16, H17, and H18, and influenza B virus Vic and Yam.

11. The method of claim 10, wherein the HA antigen, or fragment thereof, is selected from the group consisting of a full length HA antigen or a fragment thereof, HA-stalk domain or a fragment thereof, HA-head domain or a fragment thereof, and any combination thereof.

12. The method of claim 10, comprising nucleoside-modified RNA molecules encoding HA antigens from each of influenza A virus H1, H2, H3, H4, H5, H6, H7, H8, H9, H10, H11, H12, H13, H14, H15, H16, H17, and H18, and influenza B virus Vic and Yam.

13. The method of claim 10, further comprising at least one additional viral antigen.

14. The method of claim 10, wherein the composition comprises a combination of mRNA molecules encoded by nucleotide sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO: 15, SEQ ID NO:16, SEQ ID NO:17, SEQ ID NO:18, SEQ ID NO:19 and SEQ ID NO: 20.

15. The method of claim 10, wherein the composition further comprises an adjuvant.

16. The method of claim 10, wherein the nucleoside-modified RNA molecules are encapsulated within the LNPs.

17. The method of claim 10, wherein the nucleoside-modified RNA molecules comprise at least one modified nucleoside selected from the group consisting of pseudouridine, 1-methyl pseudouridine, and 5-methyl-uridine.

18. The method of claim 10, wherein the composition is administered by a delivery route selected from the group consisting of intradermal, subcutaneous, inhalation, intranasal, and intramuscular.

19. The method of claim 10, wherein the method comprises a single administration of the composition.

20. The method of claim 10, wherein the method comprises multiple administrations of the composition.
